(19) United States
(12) Patent Application Publication
(10) Pub. No.: US 2026/0216342 A1
(43) Pub. Date: Jul. 30, 2026

(54) COMPOUNDS FOR NUCLEIC ACID CLEAVAGE

(71) Applicant: Cambridge Enterprise Limited, Cambridge (GB)

(72) Inventors: Gonçalo Bernardes, Cambridge (GB); Sigitas Mikutis, Cambridge (GB); Rupert Proctor, Cambridge (GB); Yuliia Vyborna, Cambridge (GB); Anna Sib, Cambridge (GB)

(21) Appl. No.: 19/148,745

(22) PCT Filed: Jan. 19, 2024
(86) PCT No.: PCT/GB2024/050151

§ 371 Date: Jul. 17, 2025

(30) Foreign Application Priority Data
Jan. 20, 2023 (GB) 2300884.0

(51) Int. Cl. A61K 47/54; A61K 47/55; C12Q 1/6806

(52) U.S. Cl. CPC A61K 47/545; A61K 47/547; A61K 47/55; C12Q 1/6806

(57) ABSTRACT

The present invention relates to novel compounds of formula (I) defined herein that are suitable for the non-enzymatic cleavage of target nucleic acids. The present invention also relates to the pharmaceutical compositions comprising these compounds and to the use of these compounds in, for example, epigenomic and epitranscriptomic mapping, as well as in therapy, such as anti-microbial and/or anti-viral therapy.

The project leading to this application has received funding from the European Union's Horizon 2020 research and innovation program under the European Research Council grant agreement No 676832.

### FIELD OF INVENTION

The present invention relates to novel compounds suitable for the non-enzymatic cleavage of target nucleic acids. The present invention also relates to the use of these compounds in, for example, RNA structure mapping, as well as in therapy, such as anti-microbial and/or anti-viral therapy.

### BACKGROUND

Targeted degradation of nucleic acids forms part of the cutting edge in drug development. The destruction of ribonucleic acid (RNA) strands in living systems is critical to effective biological function in an organism. Crucial to a broad range of functions, RNAs represent important targets for disruption of disease. Established methods for targeted degradation utilise cellular co-factors, complicating their potential deployment. Additionally, the specificity of this approach carries an inflexibility in the compounds that can be used.

In 2019, a respiratory illness changed the way life is lived. The virus that causes the illness, known as COVID-19 or SARS-CoV-2, has precipitated our observed need to develop medicines capable of targeting and destroying viral machinery. Beyond covalent modifications, the secondary and tertiary structure of RNA complexes can also have a pathological effect and therefore be exploited as a focus for therapy. The genome of SARS-CoV-2 contains four putative G-Quadruplex sites [Zhao, C., et al.].

Mikutis et al., 2020 describes a small molecule “click degrader” that can be covalently attached to an RNA species through click-chemistry and can then cleave the attached RNA molecule. The authors describe a methylation CLICK degradation sequencing method (meCLICK-Seq) for identifying the presence of N⁶-methyladenosine (m⁶A) in an RNA sequence. The method hijacks an RNA methyltransferase to introduce an alkyne moiety, instead of a methyl group, on RNA. A subsequent copper(I)-catalysed azide-alkyne cycloaddition reaction incorporates the click-degrader molecule, leading to RNA cleavage. The method identifies methylated transcripts, determines RNA methylase specificity, and reliably maps modification sites in intronic and intergenic regions.

As the click degrader molecules are covalently incorporated into the target RNA, they can only be used to degrade RNA species which can be edited to contain a suitable click-reactive group (typically an alkyne). Moreover, the required editing of the RNA limits the application of the technology to therapeutics.

Furthermore, although imidazole, the original bioinspired cleavage group (warhead) used for the meClick-seq method, demonstrated itself to be sufficient to prove the basis of the strategy, its simplicity carries with it an inherent lack of tuneability for efficacy and DMPK properties with a view to human treatment. Furthermore, because of the difference in topology for each new RNA binder when complexed, degrader efficiency may vary from target to target. As such, there is a need to generate new degrader molecules suitable for pharmaceutical applications.

The present invention was devised with the foregoing in mind.

### SUMMARY OF THE INVENTION

The present invention relates to the finding that a bifunctional compound, also referred to herein as a degrader, can be used as a catalytic agent to non-covalently bind to, and cleave, a target nucleic acid molecule. The degraders disclosed herein bind to a target nucleic acid through non-covalent interactions. Surprisingly, the inventors have found that non-covalent binding is sufficient to enable the selective degradation of the target nucleic acid. Accordingly, the degraders do not require any chemical modification of the target nucleic acid, e.g. the incorporation of a click-reactive group into the target nucleic acid.

The selective cleavage of target nucleic acid molecules using the degraders described herein may be useful in RNA structure mapping, as well as in therapy, for example anti-cancer, anti-bacterial and anti-viral therapy.

In one aspect, the present invention provides a bifunctional compound as defined herein, or a pharmaceutically acceptable salt or solvate thereof.

In another aspect, the present invention provides a pharmaceutical composition which comprises a bifunctional compound as defined herein, or a pharmaceutically acceptable salt or solvate thereof, and one or more pharmaceutically acceptable excipients.

In another aspect, the present invention provides a bifunctional compound as defined herein, or a pharmaceutically acceptable salt or solvate thereof, for use as a medicament.

In another aspect, the present invention provides a bifunctional compound as defined herein, or a pharmaceutically acceptable salt or solvate thereof, for use in the treatment of a disease or condition in which the degradation of a target oligonucleotide is beneficial.

In another aspect, the present invention provides a bifunctional compound as defined herein, or a pharmaceutically acceptable salt or solvate thereof, for use in the treatment of a proliferative disorder (e.g. cancer) or a bacterial or viral infection.

In another aspect, the present invention provides the use of a bifunctional compound as defined herein, or a pharmaceutically acceptable salt or solvate thereof, in the manufacture of a medicament for use in the treatment of a disease or condition in which the degradation of a target oligonucleotide is beneficial.

In another aspect, the present invention provides the use of a bifunctional compound as defined herein, or a pharmaceutically acceptable salt or solvate thereof, in the manufacture of a medicament for use in the treatment of a proliferative disorder (e.g. cancer) or a bacterial or viral infection.

In another aspect, the present invention provides a method of treating a disease or condition in which the degradation of a target oligonucleotide is beneficial, the method comprising administering a therapeutically effective dose of a bifunctional compound as defined herein, or a pharmaceutically acceptable salt or solvate thereof.

In another aspect, the present invention provides a method of treating a proliferative disorder (e.g. cancer) or a bacterial or viral infection, the method comprising administering a therapeutically effective dose of a bifunctional compound as defined herein, or a pharmaceutically acceptable salt or solvate thereof.

In another aspect, the present invention provides a bifunctional compound as defined herein, or a pharmaceutically acceptable salt or solvate thereof, for use in epigenetic and epitranscriptomic analysis/mapping.

In another aspect, the present invention provides the use of a bifunctional compound as defined herein, or a salt or solvate thereof, for epigenetic and epitranscriptomic analysis/mapping.

In another aspect, the present invention provides a method for cleaving a target nucleic acid molecule, the method comprising:
contacting the target nucleic acid molecule with a bifunctional compound of the present invention such that the compound non-covalently binds to the target nucleic acid molecule; and
allowing the compound to cleave the target nucleic acid molecule bound thereto.

In another aspect, the present invention provides a method for identifying a secondary or tertiary structure within a target nucleic acid molecule, the method comprising:
providing first and second populations of nucleic acid molecules, each population comprising the target nucleic acid molecule;
introducing into the first population of nucleic acid molecules a bifunctional compound of the present invention;
allowing the bifunctional compound of the present invention to cleave the target nucleic acid molecule present in the first population; and
identifying nucleic acid molecules which are present in a reduced amount in the first population relative to the second population.

The present invention further provides a method of synthesising a bifunctional compound, or a pharmaceutically acceptable salt, as defined herein.

Preferred, suitable, and optional features of any one particular aspect of the present invention are also preferred, suitable, and optional features of any other aspect.

### DETAILED DESCRIPTION OF THE INVENTION

### Definitions

Unless otherwise stated, the following terms used in the specification and claims have the following meanings set out below.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

A “therapeutically effective amount” means the amount of a bifunctional compound that, when administered to a mammal for treating a disease, is sufficient to effect such treatment for the disease. The “therapeutically effective amount” will vary depending on the compound, the disease and its severity and the age, weight, etc., of the mammal to be treated.

In this specification the term “alkyl” includes both straight and branched chain alkyl groups and analogues thereof. References to individual alkyl groups such as “propyl” are specific for the straight chain version only and references to individual branched chain alkyl groups such as “isopropyl” are specific for the branched chain version only. For example, “(1-6C)alkyl” includes (1-4C)alkyl, (1-3C)alkyl, propyl, isopropyl and t-butyl. A similar convention applies to other radicals, for example “phenyl(1-6C)alkyl” includes phenyl(1-4C)alkyl, benzyl, 1-phenylethyl and 2-phenylethyl.

The term “(m-nC)” or “(m-nC) group” used alone or as a prefix, refers to any group having m to n carbon atoms.

The term “heteroalkyl” is an alkyl group in which one or more carbon atoms is replaced with a heteroatom, for example N, O and S. A heteroalkyl group may be a 1-6C heteroalkyl group, for example, a 1-4C, 1-3C or a 1-2C heteroalkyl group. In this context, the prefix (e.g. 1-6C) denotes the number of atoms in the heteroalkyl backbone, whether carbon atoms or heteroatoms. The heteroalkyl group may be linear or branched.

An “alkylene” group is an alkyl group that is positioned between and serves to connect two other chemical groups. Thus, “(1-6C)alkylene” means a linear saturated divalent hydrocarbon radical of one to six carbon atoms or a branched saturated divalent hydrocarbon radical of three to six carbon atoms, for example, methylene, ethylene, propylene, 2-methylpropylene, pentylene, and the like.

“(3-8C)cycloalkyl” means a hydrocarbon ring containing from 3 to 8 carbon atoms, for example, cyclopropyl, cyclobutyl, cyclopentyl, cyclohexyl, cycloheptyl or bicyclo[2.2.1]heptyl.

“(3-8C)cycloalkyl-(1-6C)alkylene” means a (3-8C)cycloalkyl group covalently attached to a (1-6C)alkylene group, both of which are defined herein.

The term “halo” or “halogeno” refers to fluoro, chloro, bromo and iodo.

The term “heterocyclyl”, “heterocyclic” or “heterocycle” means a non-aromatic saturated or partially saturated monocyclic, fused, bridged, or spiro bicyclic heterocyclic ring system(s). The term heterocyclyl includes both monovalent species and divalent species. Monocyclic heterocyclic rings contain from about 3 to 12 (suitably from 3 to 7) ring atoms, with from 1 to 5 (suitably 1, 2 or 3) heteroatoms selected from nitrogen, oxygen or sulfur in the ring. Bicyclic heterocycles contain from 7 to 17 member atoms, suitably 7 to 12 member atoms, in the ring. Bicyclic heterocycles contain from about 7 to about 17 ring atoms, suitably from 7 to 12 ring atoms. Bicyclic heterocyclic(s) rings may be fused, spiro, or bridged ring systems.

Examples of heterocyclic groups include cyclic ethers such as oxiranyl, oxetanyl, tetrahydrofuranyl, dioxanyl, and substituted cyclic ethers. Heterocycles containing nitrogen include, for example, azetidinyl, pyrrolidinyl, piperidinyl, piperazinyl, tetrahydrotriazinyl, tetrahydropyrazolyl, and the like. Typical sulfur containing heterocycles include tetrahydrothienyl, dihydro-1,3-dithiol, tetrahydro-2H-thiopyran, and hexahydrothiepine. Other heterocycles include dihydro-oxathiolyl, tetrahydro-oxazolyl, tetrahydro-oxadiazolyl, tetrahydrodioxazolyl, tetrahydro-oxathiazolyl, hexahydrotriazinyl, tetrahydro-oxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothienyl and thiomorpholinyl such as tetrahydrothiene 1,1-dioxide and thiomorpholinyl 1,1-dioxide. A suitable value for a heterocyclyl group which bears 1 or 2 oxo (═O) or thioxo (═S) substituents is, for example, 2-oxopyrrolidinyl, 2-thioxopyrrolidinyl, 2-oxoimidazolidinyl, 2-thioxoimidazolidinyl, 2-oxopiperidinyl, 2,5-dioxopyrrolidinyl, 2,5-dioxoimidazolidinyl or 2,6-dioxopiperidinyl. Particular heterocyclyl groups are saturated monocyclic 3 to 7 membered heterocyclyls containing 1, 2 or 3 heteroatoms selected from nitrogen, oxygen or sulfur, for example azetidinyl, tetrahydrofuranyl, tetrahydropyranyl, pyrrolidinyl, morpholinyl, tetrahydrothienyl, tetrahydrothienyl 1,1-dioxide, thiomorpholinyl, thiomorpholinyl 1,1-dioxide, piperidinyl, homopiperidinyl, piperazinyl or homopiperazinyl. As the skilled person would appreciate, any heterocycle may be linked to another group via any suitable atom, such as via a carbon or nitrogen atom. However, reference herein to piperidino or morpholino refers to a piperidin-1-yl or morpholin-4-yl ring that is linked via the ring nitrogen.

By “bridged ring systems” is meant ring systems in which two rings share more than two atoms, see for example Advanced Organic Chemistry, by Jerry March, 4^{th} Edition, Wiley Interscience, pages 131-133, 1992. Examples of bridged heterocyclyl ring systems include, aza-bicyclo[2.2.1]heptane, 2-oxa-5-azabicyclo[2.2.1]heptane, aza-bicyclo[2.2.2]octane, aza-bicyclo[3.2.1]octane and quinuclidine.

“Heterocyclyl(1-6C)alkyl” means a heterocyclyl group covalently attached to a (1-6C)alkylene group, both of which are defined herein.

The term “heteroaryl” or “heteroaromatic” means an aromatic mono-, bi-, or polycyclic ring incorporating one or more (for example 1-4, particularly 1, 2 or 3) heteroatoms selected from nitrogen, oxygen or sulfur. The term heteroaryl includes both monovalent species and divalent species. Examples of heteroaryl groups are monocyclic and bicyclic groups containing from five to twelve ring members, and more usually from five to ten ring members. The heteroaryl group can be, for example, a 5- or 6-membered monocyclic ring or a 9- or 10-membered bicyclic ring, for example a bicyclic structure formed from fused five and six membered rings or two fused six membered rings. Each ring may contain up to about four heteroatoms typically selected from nitrogen, sulfur and oxygen. Typically the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of heteroaryl include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3-b]furanyl, 2H-furo[3,2-b]-pyranyl, 5H-pyrido[2,3-d]-o-oxazinyl, 1H-pyrazolo[4,3-d]-oxazolyl, 4H-imidazo[4,5-d]thiazolyl, pyrazino[2,3-d]pyridazinyl, imidazo[2,1-b]thiazolyl, imidazo[1,2-b][1,2,4]triazinyl. “Heteroaryl” also covers partially aromatic bi- or polycyclic ring systems wherein at least one ring is an aromatic ring and one or more of the other ring(s) is a non-aromatic, saturated or partially saturated ring, provided at least one ring contains one or more heteroatoms selected from nitrogen, oxygen or sulfur. Examples of partially aromatic heteroaryl groups include for example, tetrahydroisoquinolinyl, tetrahydroquinolinyl, 2-oxo-1,2,3,4-tetrahydroquinolinyl, dihydrobenzthienyl, dihydrobenzfuranyl, 2,3-dihydro-benzo[1,4]dioxinyl, benzo[1,3]dioxolyl, 2,2-dioxo-1,3-dihydro-2-benzothienyl, 4,5,6,7-tetrahydrobenzofuranyl, indolinyl, 1,2,3,4-tetrahydro-1,8-naphthyridinyl, 1,2,3,4-tetrahydropyrido[2,3-b]pyrazinyl and 3,4-dihydro-2H-pyrido[3,2-b][1,4]oxazinyl.

Examples of five membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups.

Examples of six membered heteroaryl groups include but are not limited to pyridyl, pyrazinyl, pyridazinyl, pyrimidinyl and triazinyl.

A bicyclic heteroaryl group may be, for example, a group selected from:
a benzene ring fused to a 5- or 6-membered ring containing 1, 2 or 3 ring heteroatoms;
a pyridine ring fused to a 5- or 6-membered ring containing 1, 2 or 3 ring heteroatoms;
a pyrimidine ring fused to a 5- or 6-membered ring containing 1 or 2 ring heteroatoms;
a pyrrole ring fused to a 5- or 6-membered ring containing 1, 2 or 3 ring heteroatoms;
a pyrazole ring fused to a 5- or 6-membered ring containing 1 or 2 ring heteroatoms;
a pyrazine ring fused to a 5- or 6-membered ring containing 1 or 2 ring heteroatoms;
an imidazole ring fused to a 5- or 6-membered ring containing 1 or 2 ring heteroatoms;
an oxazole ring fused to a 5- or 6-membered ring containing 1 or 2 ring heteroatoms;
an isoxazole ring fused to a 5- or 6-membered ring containing 1 or 2 ring heteroatoms;
a thiazole ring fused to a 5- or 6-membered ring containing 1 or 2 ring heteroatoms;
an isothiazole ring fused to a 5- or 6-membered ring containing 1 or 2 ring heteroatoms;
a thiophene ring fused to a 5- or 6-membered ring containing 1, 2 or 3 ring heteroatoms;
a furan ring fused to a 5- or 6-membered ring containing 1, 2 or 3 ring heteroatoms;
a cyclohexyl ring fused to a 5- or 6-membered heteroaromatic ring containing 1, 2 or 3 ring heteroatoms; and
a cyclopentyl ring fused to a 5- or 6-membered heteroaromatic ring containing 1, 2 or 3 ring heteroatoms.

Particular examples of bicyclic heteroaryl groups containing a six membered ring fused to a five membered ring include but are not limited to benzfuranyl, benzthiophenyl, benzimidazolyl, benzoxazolyl, benzisoxazolyl, benzthiazolyl, benzisothiazolyl, isobenzofuranyl, indolyl, isoindolyl, indolizinyl, indolinyl, isoindolinyl, purinyl (e.g., adeninyl, guaninyl), indazolyl, benzodioxolyl and pyrazolopyridinyl groups.

Particular examples of bicyclic heteroaryl groups containing two fused six membered rings include but are not limited to quinolinyl, isoquinolinyl, chromanyl, thiochromanyl, chromenyl, isochromenyl, chromanyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl groups.

“Heteroaryl(1-6C)alkyl” means a heteroaryl group covalently attached to a (1-6C)alkylene group, both of which are defined herein. Examples of heteroaralkyl groups include pyridin-3-ylmethyl, 3-(benzofuran-2-yl)propyl, and the like.

The term “aryl” means a cyclic or polycyclic aromatic ring having from 5 to 12 carbon atoms. The term aryl includes both monovalent species and divalent species. Examples of aryl groups include, but are not limited to, phenyl, biphenyl, naphthyl and the like. In particular embodiment, an aryl is phenyl.

The term “aryl(1-6C)alkyl” means an aryl group covalently attached to a (1-6C)alkylene group, both of which are defined herein. Examples of aryl-(1-6C)alkyl groups include benzyl, phenylethyl, and the like.

This specification also makes use of several composite terms to describe groups comprising more than one functionality. Such terms will be understood by a person skilled in the art. For example heterocyclyl(m-nC)alkyl comprises (m-nC)alkyl substituted by heterocyclyl.

The term “optionally substituted” refers to either groups, structures, or molecules that are substituted and those that are not substituted. The term “wherein a/any CH, CH₂, CH₃ group or heteroatom (i.e. NH) within a R¹ group is optionally substituted” suitably means that (any) one of the hydrogen radicals of the R¹ group is substituted by a relevant stipulated group.

Where optional substituents are chosen from “one or more” groups it is to be understood that this definition includes all substituents being chosen from one of the specified groups or the substituents being chosen from two or more of the specified groups.

The phrase “compound of the invention” means those compounds which are disclosed herein, both generically and specifically (e.g., compounds of formulae (I), (II), (Ill), (IV), or (V), (VI) or (VII)).

### SUMMARY OF THE FIGURES

The present invention is described with reference to the figures listed below.

FIG. 1: Schematic diagram of the mode of action of degraders. FIG. 1 demonstrate the use of the degraders in a coronaviral pseudoknot degradation strategy. The pseudoknot-degrader binds and then directly degrades the coronaviral region that contains the pseudoknot without a need for other agents.

FIG. 2: rG4 degraders cleave rG4-containing oligomers and SARS-CoV-2 genome in vitro. (a) Shows the effect of rG4 degraders on rG4-competent oligomer. Degraders cleave oligomers under conditions that facilitate rG4 formation. n=3. (b) Shows the effect of rG4 degraders on an oligomer that does not form rG4s. No degradation was observed. n=3. (c) Shows nanopore sequencing data indicating widespread degradation on SARS-CoV-2 genome upon treatment with rG4 degrader PDS-deg6 (9A) on its ORF1b. * p<0.05, ** p<0.01, *** p<0.005, n.s. not significant.

FIG. 3: In vitro preliminary findings of G4-degraders anti-SARS-CoV-2 activity. (a) Shows the inhibition of plaque forming units (PFU) on samples treated at 50 μM with PDS-deg4 (9B), PDS-deg6 (9A) and PDS-DegALK (8). (b) Provides PCR measurements of viral RNA. The results show the inhibition of viral replication by PDS-deg6 (9A) at 5 μM and 50 μM. As shown, PDS-deg6 (9A) appears to inhibit viral growth to a greater extent than PDS-deg4 (9B). (c) Shows the viability of cells after 24 hours of incubation with increasing concentrations of the G4-degrader. None of the compounds showed cytotoxicity up to 50 μM.

FIG. 4: In vivo anti-SARS-CoV-2 activity of G4-degrader. (a) Mice administered with PDS-deg4 (9B) (purple, triangular markers) showed 10% loss of body weight in the first day after infection, which stabilized between Day 1 and Day 3 before decreasing again to reach the 75% threshold on Day 5, as observed in vehicle (0.1% DMSO in water) treated animals (grey square markers). Non-infected mice treated with vehicle (0.1% DMSO in water) as a control (circular markers) as showed no decrease in body-weight. (b) Quantification of lung viral load on Day 5 by plaque assay showed decreased loads in animals treated with PDS-deg4 (96) (purple, right) in comparison to vehicle control group (grey, left). * p<0.01.

FIG. 5: MTDB-degrader (16a) cuts coronaviral pseudoknots in vitro. (a) Shows the synthetic design of MTDB-deg (16a). (b) Shows the structure of control molecule TDB-deg (16b) that features a weak pseudoknot binder and the imidazole cleavage portion. (c) Is LC-MS data showing the degradation of the pseudoknot in the presence of the degraders against control, n=3. (d) Shows gel photographs validating the activity of the pseudoknot degraders. (e) Is LCMS data showing that pseudoknot degraders loose efficiency when one of the pseudoknot stems is mutated to perturb the pseudoknot secondary structure, n=3. (f) Are gels photographs showing the degradation of native RNA extracted from SARS-CoV-2 by MTDB-deg (16a) against control. n.s.—not significant.

FIG. 6: Direct RNA Nanopore sequencing reveals the genomic loci which get degraded with MTDB-deg. (a) Shows distribution and abundance of aligned reads flanking the pseudoknot area for control—(0.1% DMSO in water) or MTDB-deg-treated SARS-CoV-2 RNA, based on alignments with minimap2. b, Distribution and abundance of aligned reads mapped exclusively on the S sgRNA region for control- or MTDB-deg-treated SARS-CoV-2 RNA, based on alignments with minimap2.

FIG. 7: Treatment with MTDB-deg has no effect on the subgenomic SARS-CoV-2 RNAs. Distribution and abundance of aligned reads mapped exclusively on the indicated sgRNA regions for control—(0.1% DMSO in water) or MTDB-deg-treated SARS-CoV-2 RNA, based on alignments with minimap2.

FIG. 8: MTDB-degrader inhibits SARS-CoV-2 replication in cells. (a), (b) Show the percentage of inhibition of viral replication normalised to vehicle control (dashed line) after incubation with increasing concentrations of the pseudoknot degrader (MTDB-deg (16a)) and control molecules (MTDB and TDB-deg (16b)). Viral replication was assessed after 24 h of infection (multiplicity of infection (MOI) of 0.05) based on E gene and Pseudoknot region RNA levels. Antiviral activity of the MTDB-deg (16a) was observed both before (a), and after infection (b), with SARS-CoV-2 at a 0.05 MOI. Mean±SD of triplicates is shown and differences between means with p<0.01 are indicated. *, p<0.05; **, p<0.01; two-tailed paired t-tests. (c) Shows that the 50% inhibitory concentration (IC₅₀) of the pseudoknot degrader MTDB-deg (16a) is lower when drug is added after infection. (d) Are photographs of cell monolayers after 4 days of incubation with supernatants from viral cultures that were treated with 6 mM of MTDB-deg (16a), MTDB and TDB-deg (16b) for 24 h. Treatment with the MTDB-deg (16a) at 6 mM for 24 h showed a decreased number of viral plaques in comparison to vehicle control, both when added before or after infection. Control molecule MTDB only showed decreased number of viral plaques when added before infection, and TDB-deg (16b) showed no decrease. (e) Cell viability assay demonstrating that none of the compounds showed cytotoxicity in VeroCCL81 cells after 24 h. (f) Shows the percentage of viral replication relative to vehicle control 24 h after removal of the degrader-containing medium. 24 h treatment with MTDB-deg (16a) 24 h compromised the ability of virus to recover from drug exposure.

FIG. 9: Dose-response curves of MTDB-deg, MTDB and TDB-deg. (a), (b) Show the 50% inhibitory concentration (IC₅₀) values for pseudoknot degrader MTDB-deg (16a) before and after infection. Control molecules (MTDB and TDB-deg (16b) did not inhibit viral replication and thus IC₅₀ values could not be determined. (c) Is a dose-response curve (as determined by PCR targeting the E gene), including a higher concentration of 18 μM showing increased IC₅₀.

FIG. 10: Viral recovery and virucidal activity after MTDB-degrader exposure. (a) Virus ability to recover after 24 h incubation with MTDB-deg (16a) and control molecules MTDB and TDB-deg (16b), as determined by qPCR targeting the pseudoknot region. Viral recovery was impaired in MTDB-deg (16b) treated samples, but not in samples treated with the control molecules MTDB and TDB-deg (16b). (b) Virucidal activity was assessed by incubating 1000 PFU of SARS-CoV-2 with compounds at 6 μM, for 1 h at 37° C. after which residual viral infectivity was determined by plaque assay. MTDB-deg (16a), MTDB and TDB-deg (16b) showed no virucidal effect on cell free virions, suggesting that the MTDB-deg (16a) antiviral activity is mediated by inhibiting virus replication in host cells and not by inactivation of cell free virions.

FIG. 11: Agarose gel analysis of ribosome degradation assay. Left: ethyl linker, no degradation visible. Centre left: diethylene glycol linker, degradation visible at 15 mM concentration. Centre right: hexaethylene glycol linker, no degradation visible. Right: control chloramphenicol, no degradation. B=Blank (no degrader), concentration degrader: 1a/1b=15 mM. 2a/2b=7.5 mM. 3a/3b=3.75 mM. 4a/4b=1.88 mM, 5a/5b=0.94 mM. 6a/6b=0.47 mM.

FIG. 12 MTDB-degrader in vivo activity against SARS-CoV-2 infection in K18-hACE2 mice. (a) Eight- to twelve-week-old female K18-hACE2-transgenic mice were intranasally infected with 10⁴ plaque-forming units (PFU) of SARS-CoV-2 and treated intranasally 1 hour pre-infection and 3 hours post-infection with MTDB-deg 16a (25 mg/kg) (n=6), MTDB (10 mg/kg, maximal dose that could administered given limited solubility) (n=3), TDB-deg 16b (25 mg/kg) (n=5) and vehicle control (n=6). (b) Administration of MTDB-deg 16a leads to a decrease in lung viral load of SARS-CoV-2 infected K18-hACE2 mice. No differences in lung viral load between vehicle control and MTDB and TDB-deg 16b treated mice were observed. Mean±SD is shown; *p<0.05; unpaired t-test. (c) Western blot analysis of phospho-p38 from lung extracts of transgenic K18-hACE2 mice treated with three doses of 10 mg/kg of vehicle (V1, V2) or MTDB-deg 16a (D1, D2) at 1 hour before infection and 1 and 2 days after infection (n=2).

FIG. 13 Evaluation of cleavage group/warhead efficacy using an in vitro assay. n=3, **** p<0.0001, ***p<0.001, **p<0.01, *p<0.05, ns not significant.

FIG. 14. Comparison of RNA degrader warhead potencies after incubating the degrader-functionalised RNA at 37° C. for 4 hours. (a) Degrader cleavage groups compared to warheadless linker (degrader 2) using one-sided t-test. (b) Degrader cleavage groups compared to imidazole degrader 1 using two-sided t-test. n=3, **** p<0.0001, ***p<0.001, **p<0.01, *p<0.05, ns not significant.

FIG. 15. Comparison of non-covalent PDS-based RNA degraders PDS-deg6 and PDS-Amimi after incubating with RNA at 37° C. for 4 hours. Statistical significance calculated using one-sided t-test. n=3, ***p<0.001, **p<0.01.

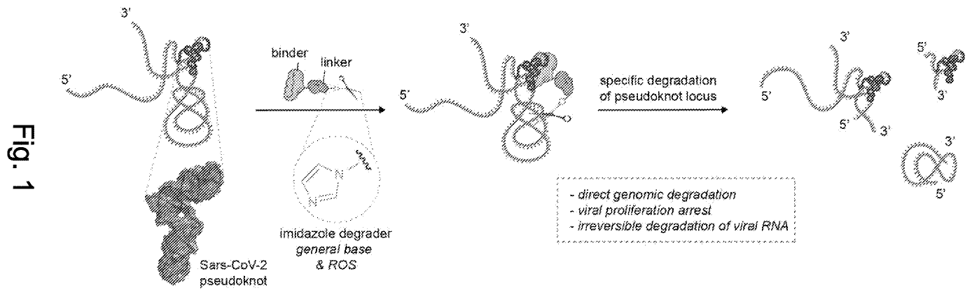

### BIFUNCTIONAL COMPOUNDS OF THE INVENTION

The present invention relates to the finding that a bifunctional compound, also known herein as a degrader, can be used as a catalytic agent to non-covalently bind to and cleave a target nucleic acid molecule. The degrader disclosed herein binds to a target nucleic acid through non-covalent interactions. Accordingly, the degrader does not require incorporation of any reactive group into the target nucleic acid that is required if a covalent interaction is desired. The selective cleavage of target nucleic acid molecules using the degraders described herein may be useful in epigenetic and epitranscriptomic analysis, bifunctional mapping, as well as in therapy, for example anti-cancer, anti-bacterial and anti-viral therapy.

In one aspect, the present invention relates to a bifunctional compound of formula (I) or a pharmaceutically acceptable salt or solvate thereof:

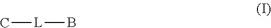

wherein:
C is a cleavage group as defined herein;
L is a linker; and
B is a non-covalent binding group.

### The Cleavage Group C

The cleavage group may be any suitable group that is capable of reacting with a target nucleic acid molecule and causing the nucleic acid molecule to cleave.

Without wishing to be bound by any particular theory, the cleavage group may act by abstracting a proton from the 2′OH position of the target nucleic acid molecule and/or the cleavage group may form a complex with copper to induce copper-mediated nucleic acid degradation (Li, Zhong-Rui, et al. Nat Chem 11.10 (2019): 880-889; Wong, K, et al. Can J Biochem 52.11 (1974): 950-958; Subramaniam, Siddharth, et al. F1000Research 4 (2015)).

The basicity of a group may be quantitatively assessed using the pKa of the associated conjugate acid. That is, the basicity of basic group [C] may be assessed using the pKa of the conjugate acid [CH]⁺. The pKa of the conjugate acid may be known or it may be determined using standard techniques, such as acid-base titration. Without wishing to be bound by theory, the inventors believe the basic residues having a conjugate acid with a pKa value above a certain threshold, such as a pKa of 5.5 or greater, 6.0 or greater, 6.2 or greater, 6.5 or greater, or 6.8 or greater, are capable of deprotonating the hydroxyl group at the 2′ position of a ribose sugar in order to permit cleavage of the phosphodiester backbone within a target nucleic acid. Suitably, the cleavage group has a pKa within the range of 5.5 to 9, more suitably 6 to 9 and most suitably 6.2 to 8.6.

Alternatively, and/or in addition, the cleavage group may be a group known to form a chelation complex with copper that is capable of inducing copper-mediated nucleic acid degradation.

Suitably, the cleavage group comprises a basic nitrogen atom or hydroxy group that either: (i) has a pKa within the range of 5.5 to 9, or 6 to 9, or 6.2 to 8.6; and/or (ii) is a nitrogen atom or hydroxy group that is capable of forming chelation complex with copper (and is thereby capable of inducing copper-mediated nucleic acid degradation).

Suitably, the basic nitrogen atom or hydroxy group is not sterically hindered by, for example, substituents (e.g. alkyl substituents) present on a carbon atom that is directly attached to the nitrogen or COH group. In an embodiment of the invention, the carbon atoms adjacent to a basic N atom or C—OH group with a pKa within the range of 5.5 to 9, or 6 to 9, or 6.2 to 8.6, is not substituted.

Suitably, the cleavage group is not:
(i) an imidazole (1,3-diazole) group that is optionally substituted with one, two or three (1-6C)alkyl groups, which may be the same or different; or
(ii) a nucleic acid cleavage group of the formula Z:

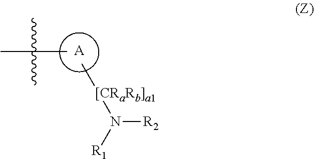

wherein:
[symbol] denotes the point of attachment to L;
Ring A is absent or a nitrogen-containing heteroaryl or heterocyclic ring which is optionally further substituted with one or more substituent groups selected from halo, trifluoromethyl, trifluoromethoxy, cyano, hydroxyl, (1-4C)alkyl, (1-4C)haloalkyl, (1-4C)hydroxyalkyl, OR_{c}, C(O)R_{c}, C(O)OR_{c}, OC(O)Rₑ, C(O)N(R_{d})R_{c}, N(R_{d})C(O)R_{c}, S(O)_{y}R_{c} (wherein y is 0, 1 or 2), SO₂N(R_{d})R_{c}, N(R_{d})SO₂R_{c}, or NR_{c}R_{d}, wherein Rₑ and R_{d} are selected from hydrogen or (1-4C)alkyl;
integer a1 is 0, 1, 2 or 3;
Ra and Rb are each independently selected at each occurrence from hydrogen or (1-2C)alkyl;
R₁ and R₂ are each independently selected from hydrogen, (1-6C)alkyl, heterocyclic ring, heterocycle-(1-3C)alkyl, heteroaryl, heteroaryl-(1-3C)alkyl, (3-6C)cycloalkyl, or (3-6C)cycloalkyl-(1-3C)alkyl, each of which is optionally substituted with one or more substituent groups selected from halo, trifluoromethyl, trifluoromethoxy, cyano, hydroxyl, ORₑ, C(O)Rₑ, C(O)ORₑ, OC(O)Rₑ, C(O)N(R_{f})Rₑ, N(R_{f})C(O)Rₑ, S(O)_{y}Rₑ (wherein y is 0, 1 or 2), SO₂N(R_{f})Rₑ, N(R_{f})SO₂Rₑ, or NRₑR_{f}, wherein Rₑ and R_{f} are selected from hydrogen or (1-4C)alkyl, or
R₁ and R₂ are linked such that, together with the atoms to which they are attached, they form a 4-6 membered heterocycle or a 5- or 6-membered heteroaryl, wherein any 4-6 membered heterocycle or 5- or 6-membered heteroaryl is optionally substituted with one or more substituent groups selected from halo, trifluoromethyl, trifluoromethoxy, cyano, hydroxyl, (1-4C)alkyl, (1-4C)haloalkyl, (1-4C)hydroxyalkyl, OR_{g}, C(O)R_{g}, C(O)OR_{g}, OC(O)R_{g}, C(O)N(Rₕ)R_{g}, N(Rₕ)C(O)R_{g}, S(O)_{y}R_{g} (wherein y is 0, 1 or 2), SO₂N(Rₕ)R_{g}, N(Rₕ)SO₂R_{g}, or NR_{g}Rₕ, wherein R_{g} and Rₕ are selected from hydrogen or (1-4C)alkyl;
wherein, when ring A is absent, R₁ and R₂ are each independently selected from hydrogen, heterocyclic ring, heterocycle-(1-3C)alkyl, heteroaryl, heteroaryl-(1-3C)alkyl, (3-6C)cycloalkyl, or (3-6C)cycloalkyl-(1-3C)alkyl, each of which is optionally substituted with one or more substituent groups selected from halo, trifluoromethyl, trifluoromethoxy, cyano, hydroxyl, ORₑ, C(O)Rₑ, C(O)ORₑ, OC(O)Rₑ, C(O)N(R_{f})Rₑ, N(R_{f})C(O)Rₑ, S(O)_{y}Rₑ (wherein y is 0, 1 or 2), SO₂N(R_{f})Rₑ, N(R_{f})SO₂Rₑ, or NRₑR_{f}, wherein Rₑ and R_{f} are selected from hydrogen or (1-4C)alkyl, provided that R₁ and R₂ are not both hydrogen.

Suitably, the cleavage group is not:
(i) an imidazole (1,3-diazole) group that is optionally substituted with one, two or three (1-6C)alkyl groups, which may be the same or different; or
(ii) a nucleic acid cleavage group of the formula Z:

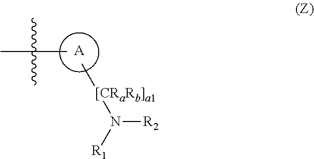

wherein:
[symbol] denotes the point of attachment to L;
Ring A is a nitrogen-containing heteroaryl or heterocyclic ring which is optionally further substituted with one or more substituent groups selected from halo, trifluoromethyl, trifluoromethoxy, cyano, hydroxyl, (1-4C)alkyl, (1-4C)haloalkyl, (1-4C)hydroxyalkyl, OR_{c}, C(O)R_{c}, C(O)OR_{c}, OC(O)R_{c}, C(O)N(R_{d})R_{c}, N(R_{d})C(O)R_{c}, S(O)_{y}R_{c} (wherein y is 0, 1 or 2), SO₂N(R_{d})R_{c}, N(R_{d})SO₂R_{c}, or NR_{c}R_{d}, wherein R, and R_{d} are selected from hydrogen or (1-4C)alkyl;
integer a1 is 0, 1, 2 or 3;
Ra and Rb are each independently selected at each occurrence from hydrogen or (1-2C)alkyl;
R₁ and R₂ are each independently selected from hydrogen, (1-6C)alkyl, (3-6C)cycloalkyl, or (3-6C)cycloalkyl-(1-3C)alkyl, each of which is optionally substituted with one or more substituent group selected from halo, trifluoromethyl, trifluoromethoxy, cyano, hydroxyl, ORₑ, C(O)Rₑ, C(O)ORₑ, OC(O)Rₑ, C(O)N(R_{f})Rₑ, N(R_{f})C(O)Rₑ, S(O)_{y}Rₑ (wherein y is 0, 1 or 2), SO₂N(R_{f})Rₑ, N(R_{f})SO₂Rₑ, or NRₑR_{f}, wherein Rₑ and R_{f} are selected from hydrogen or (1-4C)alkyl.

Particular compounds of the invention include, for example, compounds of Formula (I) or any sub-formula thereof, or pharmaceutically acceptable salts and/or solvates thereof, wherein, unless otherwise stated, the cleavage group C, and any associated substituent groups has any of the meanings defined hereinbefore or in any of paragraphs (1) to (12) hereinafter:
(1) the cleavage group C is selected from:
   (i) any N or C—OH containing moiety whereby at least one N or C—OH group has a pKa within the range of 5.5 to 9, or 6 to 9, or 6.2 to 8.6;
   (ii) any N or OH containing moiety that is capable of chelating to a metal, e.g. copper or zinc, at physiological pH;
   with the proviso that the cleavage group is not:
      a) an imidazole (1,3-diazole) group that is optionally substituted with one, two or three (1-6C)alkyl groups, which may be the same or different; or
      b) a group of the formula Z defined above;
(2) the cleavage group C is a group of the formula:

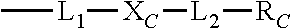

wherein:
   L₁ is absent or (1-6C)alkylene; X_{C} is absent or selected from —O—, —S—, —SO—, —SO₂—, —N(R_{XC1})—, —C(O)—, —C(O)O—, —OC(O)—, —C(O)N(R_{XC1})—, —N(R_{XC1})C(O)—, —N(R_{XC2})C(O)N(R_{XC1})—, —N(R_{XC1})C(O)O—, —OC(O)N(R_{XC1})—, —S(O)₂N(R_{XC1}), —N(R_{XC1})SO₂—, —C(O)N(R_{XC1})SO₂— or —SO₂N(R_{XC1})C(O)—; and wherein R_{XC1} and R_{XC2} are each independently selected from hydrogen or (1-6C)alkyl, (3-6C)cycloalkyl, (3-6C)cycloalkyl(1-2C)alkylene, —(CH₂)ₘ₁-aryl, —(CH₂)ₘ₁-heteroaryl or —(CH₂)ₘ₁-heterocyclic ring, wherein m1 is 0 to 4;
   L₂ is absent or (1-6C)alkylene;
   R_{C} is selected from hydrogen, (1-6C)alkyl, cycloalkyl, aryl, heteroaryl or heterocyclyl;
   wherein:
      an alkyl, cycloalkyl or aryl, is substituted by one or more R_{A} substituents and is optionally further substituted by one or more R_{B} substituents;
      a heteroaryl or heterocyclic ring is optionally substituted by one or more R_{A} and/or R_{B} substituents;
   R_{A} is a group selected from:
      (i) —OH;
      (ii) (1-6C)hydroxyalkyl;
      (iii) (3-6C)hydroxycycloalkyl;
      (iv) NR_{A1}R_{A2};
      (v) -(1-6C)alkylene-NR_{A1}R_{A2};
      (vi) -(3-6C)cycloalkylene-NR_{A1}R_{A2};
      (vii) —(CH₂)ₘ₂—R_{A3}; wherein m2 is 0 to 6;
   wherein:
      R_{A1} and R_{A2} are each independently selected from hydrogen, (1-6C)alkyl, or (1-6C)heteroalkyl;
      R_{A3} is cycloalkyl or aryl optionally substituted by one or more OH or NR_{A1}R_{A2} substituents and is optionally further substituted by one or more R_{B} substituents; or heteroaryl or heterocyclyl optionally substituted by one or more OH or NR_{A1}R_{A2} or R_{B} substituents;
   R_{B} is selected from halo, nitro, cyano, R_{BA}, —[CH₂]ₜ—OR_{BA}, —[CH₂]ₜ—C(O)R_{BA}, —[CH₂]ₜ—C(O)OR_{BA}, —[CH₂]ₜ—OC(O)R_{BA}, —[CH₂]ₜ—C(O)N(R_{BB})R_{BA}, —[CH₂]ₜ—N(R_{BB})C(O)R_{BA}, —[CH₂]ₜ—S(O)ₚR_{BA} (where p is 0, 1 or 2), —[CH₂]ₜ—SO₂N(R_{BB})R_{BA}, or —[CH₂]ₜ—N(R_{BB})SO₂R_{BA};
      wherein t is 0, 1, 2 or 3;
      R_{BA} is hydrogen or (1-4C)alkyl optionally substituted by halo, hydroxy, amino or cyano; and
      R_{BB} is hydrogen or (1-2C)alkyl;
wherein the cleavage group is not:
   a) an imidazole (1,3-diazole) group that is optionally substituted with one, two or three (1-6C)alkyl groups, which may be the same or different; or
   b) a group of the formula Z defined above;
(2A) the cleavage group C is a group of the formula:

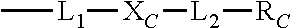

wherein:
   L₁ is absent or (1-6C)alkylene;
   X_{C} is absent or selected from —O—, —S—, —SO—, —SO₂—, —N(R_{XC1})—, —C(O)—, —C(O)O—, —OC(O)—, —C(O)N(R_{XC1})—, —N(R_{XC1})C(O)—, —N(R_{XC2})C(O)N(R_{XC1})—, —N(R_{XC1})C(O)O—, —OC(O)N(R_{XC1})—, —S(O)₂N(R_{XC1}), —N(R_{XC1})SO₂—, —C(O)N(R_{XC1})SO₂— or —SO₂N(R_{XC1})C(O)—; and wherein R_{XC1} and R_{XC2} are each independently selected from hydrogen or (1-6C)alkyl, (3-6C)cycloalkyl, (3-6C)cycloalkyl(1-2C)alkylene, —(CH₂)ₘ₁-aryl, —(CH₂)ₘ₁-heteroaryl or —(CH₂)ₘ₁-heterocyclic ring, wherein m1 is 0 to 4;
   L₂ is absent or (1-6C)alkylene;
   R_{C} is selected from hydrogen, (1-6C)alkyl, cycloalkyl, aryl, heteroaryl or heterocyclyl;
   wherein:
      an alkyl, cycloalkyl or aryl, is substituted by one or more R_{A} substituents and is optionally further substituted by one or more R_{B} substituents;
      a heteroaryl or heterocyclic ring is optionally substituted by one or more R_{A} and/or R_{B} substituents;
   R_{A} is a group selected from:
      (i) —OH;
      (ii) (1-6C)hydroxyalkyl;
      (iii) (3-6C)hydroxycycloalkyl;
      (iv) -(3-6C)cycloalkylene-NR_{A1}R_{A2};
      (v) —(CH₂)ₘ₂—R_{A3}; wherein m2 is 0 to 6;
   wherein:
      R_{A1} and R_{A2} are each independently selected from hydrogen, (1-6C)alkyl, or (1-6C)heteroalkyl;
      R_{A3} is cycloalkyl or aryl optionally substituted by one or more OH or NR_{A1}R_{A2} substituents and is optionally further substituted by one or more R_{B} substituents; or heteroaryl or heterocyclyl optionally substituted by one or more OH or NR_{A1}R_{A2} or R_{B} substituents;
   R_{B} is selected from halo, nitro, cyano, R_{BA}, —[CH₂]ₜ—OR_{BA}, —[CH₂]ₜ—C(O)R_{BA}, —[CH₂]ₜ—C(O)OR_{BA}, —[CH₂]ₜ—OC(O)R_{BA}, —[CH₂]ₜ—C(O)N(R_{BB})R_{BA}, —[CH₂]ₜ—N(R_{BB})C(O)R_{BA}, —[CH₂]ₜ—S(O)_{P}R_{BA} (where p is 0, 1 or 2), —[CH₂]ₜ—SO₂N(R_{BB})R_{BA}, or —[CH₂]ₜ—N(R_{BB})SO₂R_{BA};
      wherein t is 0, 1, 2 or 3;
      R_{BA} is hydrogen or (1-4C)alkyl optionally substituted by halo, hydroxy, amino or cyano; and
      R_{BB} is hydrogen or (1-2C)alkyl;
wherein when L₁, X_{C} and L₂ are absent, and R_{C} is a nitrogen-containing heteroaryl or heterocyclic ring it is substituted by R_{A} above; and wherein the cleavage group is not:
   a) an imidazole (1,3-diazole) group that is optionally substituted with one, two or three (1-6C)alkyl groups, which may be the same or different; or
   b) a group of the formula Z defined above;
(3) the cleavage group C is a group of the formula:

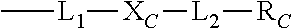

wherein:
   L₁ is absent or (1-2C)alkylene; X_{C} is absent or selected from —O—, —S—, —SO—, —SO₂—, —N(R_{XC1})—, —C(O)—, —C(O)O—, —OC(O)—, —C(O)N(R_{XC1})—, —N(R_{XC1})C(O)—, —S(O)₂N(R_{XC1}), or —N(R_{XC1})SO₂—; and wherein R_{XC1} is selected from hydrogen or (1-6C)alkyl, (3-6C)cycloalkyl, (3-6C)cycloalkyl(1-2C)alkylene, —(CH₂)ₘ₁-aryl, —(CH₂)m-heteroaryl or —(CH₂)ₘ₁-heterocyclic ring, wherein m1 is 0 to 2;
   L₂ is absent or (1-2C)alkylene;
   R_{C} is selected from hydrogen, (1-4C)alkyl, (3-6C)cycloalkyl, phenyl, heteroaryl or heterocyclyl;
   wherein:
      an alkyl, cycloalkyl, phenyl, is substituted by one or more R_{A} substituents and is optionally further substituted by one or more R_{B} substituents;
      a heteroaryl or heterocyclic ring is optionally substituted by one or more R_{A} and/or R_{B} substituents;
   R_{A} is a group selected from:
      (i) —OH;
      (ii) NR_{A1}R_{A2};
      (iii) -(1-6C)alkylene-NR_{A1}R_{A2};
      wherein:
         R_{A1} and R_{A2} are each independently selected from hydrogen, (1-6C)alkyl, or (1-6C)heteroalkyl;
   R_{B} is selected from halo, nitro, cyano, R_{BA}, —[CH₂]ₜ—OR_{BA}, —[CH₂]ₜ—C(O)R_{BA}, —[CH₂]ₜ—C(O)OR_{BA}, —[CH₂]ₜ—OC(O)R_{BA}, —[CH₂]ₜ—C(O)N(R_{BB})R_{BA}, —[CH₂]ₜ—N(R_{BB})C(O)R_{BA}, —[CH₂]ₜ—S(O)ₚR_{BA} (where p is 0, 1 or 2), —[CH₂]ₜ—SO₂N(R_{BB}) R_{BA}, or —[CH₂]ₜ—N(R_{BB})SO₂R_{BA};
      wherein t is 0, 1, 2 or 3;
      R_{BA} is hydrogen or (1-4C)alkyl optionally substituted by halo, hydroxy, amino or cyano; and
      R_{BB} is hydrogen or (1-2C)alkyl;
wherein the cleavage group is not:
   a) an imidazole (1,3-diazole) group that is optionally substituted with one, two or three (1-6C)alkyl groups, which may be the same or different; or
   b) a group of the formula Z defined above;
(3A) the cleavage group C is a group of the formula:

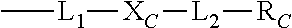

wherein:
   L₁ is absent or (1-2C)alkylene;
   X_{C} is absent or selected from —O—, —S—, —SO—, —SO₂—, —N(R_{XC1})—, —C(O)—, —C(O)O—, —OC(O)—, —C(O)N(R_{XC1})—, —N(R_{XC1})C(O)—, —S(O)₂N(R_{XC1}), or —N(R_{XC1})SO₂—; and
   wherein R_{XC1} is selected from hydrogen or (1-6C)alkyl, (3-6C)cycloalkyl, (3-6C)cycloalkyl(1-2C)alkylene, —(CH₂)ₘ₁-aryl, —(CH₂)ₘ₁-heteroaryl or —(CH₂)ₘ₁-heterocyclic ring, wherein m1 is 0 to 2;
   L₂ is absent or (1-2C)alkylene;
   R_{C} is selected from hydrogen, (1-4C)alkyl, (3-6C)cycloalkyl, phenyl, heteroaryl or heterocyclyl;
   wherein:
      an alkyl, cycloalkyl, phenyl, is substituted by one or more R_{A} substituents and is optionally further substituted by one or more R_{B} substituents;
      a heteroaryl or heterocyclic ring is optionally substituted by one or more R_{A} and/or R_{B} substituents;
   R_{A} is a group selected from:
      (i) —OH;
      (ii) (1-6C)hydroxyalkyl;
      (iii) (3-6C)hydroxycycloalkyl;
      (iv) -(3-6C)cycloalkylene-NR_{A1}R_{A2};
      wherein:
         R_{A}, and R_{A2} are each independently selected from hydrogen, (1-6C)alkyl, or (1-6C)heteroalkyl;
   R_{B} is selected from halo, nitro, cyano, R_{BA}, —[CH₂]ₜ—OR_{BA}, —[CH₂]ₜ—C(O)R_{BA}, —[CH₂]ₜ—C(O)OR_{BA}, —[CH₂]ₜ—OC(O)R_{BA}, —[CH₂]ₜ—C(O)N(R_{BB})R_{BA}, —[CH₂]ₜ—N(R_{BB})C(O)R_{BA}, —[CH₂]ₜ—S(O)ₚR_{BA} (where p is 0, 1 or 2), —[CH₂]ₜ—SO₂N(R_{BB}) R_{BA}, or —[CH₂]ₜ—N(R_{BB})SO₂R_{BA};
      wherein t is 0, 1, 2 or 3;
      R_{BA} is hydrogen or (1-4C)alkyl optionally substituted by halo, hydroxy, amino or cyano; and
      R_{BB} is hydrogen or (1-2C)alkyl;
wherein when L₁, X_{C} and L₂ are absent, and R_{C} is a nitrogen-containing heteroaryl or heterocyclic ring it is substituted by R_{A} above; and wherein the cleavage group is not:
   a) an imidazole (1,3-diazole) group that is optionally substituted with one, two or three (1-6C)alkyl groups, which may be the same or different; or
   b) a group of the formula Z defined above;
(4) the cleavage group C is a group of the formula:

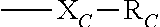

wherein:
   X_{C} is absent or selected from —O—, —S—, —SO—, —SO₂—, —N(R_{XC1})—, —C(O)—, —C(O)N(R_{XC1})— or —N(R_{XC1})C(O)—; and wherein R_{XC1} is selected from hydrogen or (1-6C)alkyl or —(CH₂)ₘ₁-heteroaryl, wherein m1 is 0 to 2;
   R_{C} is selected from hydrogen, (1-4C)alkyl, phenyl, heteroaryl or heterocyclyl;
   wherein:
      the alkyl, cycloalkyl, phenyl, is substituted by one or more R_{A} substituents and is optionally further substituted by one or more R_{B} substituents;
      a heteroaryl or heterocyclic ring is optionally substituted by one or more R_{A} and/or R_{B} substituents;
   R_{A} is a group selected from:
      (i) —OH;
      (ii) NR_{A1}R_{A2};
      (iii) -(1-6C)alkylene-NR_{A1}R_{A2};
      wherein:
         R_{A}, and R_{A2} are each independently selected from hydrogen, (1-6C)alkyl, or (1-6C)heteroalkyl;
   R_{B} is selected from halo, nitro, cyano, R_{BA}—[CH₂]ₜ—OR_{BA}, or —[CH₂]ₜ—C(O)OR_{BA},
      wherein t is 0, 1, 2 or 3; and
      R_{BA} is (1-4C)alkyl;
wherein the cleavage group is not:
   a) an imidazole (1,3-diazole) group that is optionally substituted with one, two or three (1-6C)alkyl groups, which may be the same or different; or
   b) a group of the formula Z defined above;
(4A) the cleavage group C is a group of the formula:

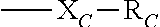

wherein:
   X_{C} is absent or selected from —O—, —S—, —SO—, —SO₂—, —N(R_{XC1})—, —C(O)—, —C(O)N(R_{XC1})— or —N(R_{XC1})C(O)—; and wherein R_{XC1} is selected from hydrogen or (1-6C)alkyl or —(CH₂)ₘ₁-heteroaryl, wherein m1 is 0 to 2;
   R_{C} is selected from hydrogen, (1-4C)alkyl, phenyl, heteroaryl or heterocyclyl;
   wherein:
      an alkyl or phenyl is substituted by one or more R_{A} substituents and is optionally further substituted by one or more R_{B} substituents;
      a heteroaryl or heterocyclic ring is optionally substituted by one or more R_{A} and/or R_{B} substituents;
   R_{A} is a group selected from:
      (i) —OH;
      (ii) (1-6C)hydroxyalkyl;
   R_{B} is selected from halo, nitro, cyano, R_{BA}—[CH₂]ₜ—OR_{BA}, or —[CH₂]ₜ—C(O)OR_{BA},
      wherein t is 0, 1, 2 or 3; and
      R_{BA} is (1-4C)alkyl;
wherein when X_{C} is absent, and R_{C} is a nitrogen-containing heteroaryl or heterocyclic ring it is substituted by R_{A} above; and
wherein the cleavage group is not:
   a) an imidazole (1,3-diazole) group that is optionally substituted with one, two or three (1-6C)alkyl groups, which may be the same or different; or
   b) a group of the formula Z defined above;
(5) the cleavage group C is a group of the formula:

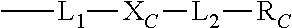

wherein:
   L₁ is absent or (1-6C)alkylene;
   X_{C} is absent or selected from —O—, —S—, —SO—, —SO₂—, —N(R_{XC1})—, —C(O)—, —C(O)O—, —OC(O)—, —C(O)N(R_{XC1})—, —N(R_{XC1})C(O)—, —N(R_{XC2})C(O)N(R_{XC1})—, —N(R_{XC1})C(O)O—, —OC(O)N(R_{XC1})—, —S(O)₂N(R_{XC1}), —N(R_{XC1})SO₂—, —C(O)N(R_{XC1})SO₂— or —SO₂N(R_{XC1})C(O)—; and wherein R_{XC1} and R_{XC2} are each independently selected from hydrogen or (1-6C)alkyl, (3-6C)cycloalkyl, (3-6C)cycloalkyl(1-2C)alkylene, —(CH₂)ₘ₁-aryl, —(CH₂)ₘ₁-heteroaryl or —(CH₂)ₘ₁-heterocyclic ring, wherein m1 is 0 to 4;
   L₂ is absent or (1-6C)alkylene;
   R_{C} is selected from hydrogen, (1-6C)alkyl, cycloalkyl, aryl, heteroaryl or heterocyclyl;
   wherein:
      an alkyl, cycloalkyl, aryl, is substituted by one or more R_{A} substituents and is optionally further substituted by one or more R_{B} substituents if the heteroaryl or heterocyclic ring does not comprise one or more N atoms having a pKa of 5.5 to 9, or 6 to 9, or 6.2 to 8.6, then the ring is substituted by one or more R_{A} substituents and is optionally substituted by one or more R_{B} substituents, or if the heteroaryl or heterocyclic ring does comprise one or more N atoms having a pKa of 5.5 to 9, or 6 to 9, or 6.2 to 8.6, then the ring is optionally substituted by one or more R_{A} or R_{B} substituents;
   R_{A} is a group selected from:
      (i) —OH;
      (ii) (1-6C)hydroxyalkyl;
      (iii) (3-6C)hydroxycycloalkyl;
      (iv) NR_{A1}R_{A2};
      (v) -(1-6C)alkylene-NR_{A1}R_{A2};
      (vi) -(3-6C)cycloalkylene-NR_{A1}R_{A2};
      (vii) —(CH₂)ₘ₂—R_{A3}; wherein m2 is 0 to 6;
   wherein:
      R_{A1} and R_{A2} are each independently selected from hydrogen, (1-6C)alkyl, or (1-6C)heteroalkyl;
      R_{A3} is cycloalkyl or aryl optionally substituted by one or more OH or NR_{A},R_{A2} substituents and is optionally further substituted by one or more R_{B} substituents; or heteroaryl or heterocyclyl optionally substituted by one or more OH or NR_{A1}R_{A2} or R_{B} substituents;
   R_{B} is selected from halo, nitro, cyano, R_{BA}, —[CH₂]ₜ—OR_{BA}, —[CH₂]ₜ—C(O)R_{BA}, —[CH₂]ₜ—C(O)OR_{BA}, —[CH₂]ₜ—OC(O)R_{BA}, —[CH₂]ₜ—C(O)N(R_{BB})R_{BA}, —[CH₂]ₜ—N(R_{BB})C(O)R_{BA}, —[CH₂]ₜ—S(O)ₚR_{BA} (where p is 0, 1 or 2), —[CH₂]ₜ—SO₂N(R_{Bb})R_{BA}, or —[CH₂]ₜ—N(R_{BB})SO₂R_{BA};
      wherein t is 0, 1, 2 or 3;
      R_{BA} is hydrogen or (1-4C)alkyl optionally substituted by halo, hydroxy, amino or cyano; and
      R_{BB} is hydrogen or (1-2C)alkyl;
wherein the cleavage group is not:
   a) an imidazole (1,3-diazole) group that is optionally substituted with one, two or three (1-6C)alkyl groups, which may be the same or different; or
   b) a group of the formula Z defined above;
(6) the cleavage group C is a group of the formula:

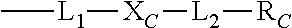

wherein:
   L₁ is absent or (1-2C)alkylene;
   X_{C} is absent or selected from —O—, —S—, —SO—, —SO₂—, —N(R_{XC1})—, —C(O)—, —C(O)O—, —OC(O)—, —C(O)N(R_{XC1})—, —N(R_{XC1})C(O)—, —S(O)₂N(R_{XC1}), or —N(R_{XC1})SO₂—; and wherein R_{XC1} is selected from hydrogen or (1-6C)alkyl, (3-6C)cycloalkyl, (3-6C)cycloalkyl(1-2C)alkylene, —(CH₂)ₘ₁-aryl, —(CH₂)ₘ₁-heteroaryl or —(CH₂)ₘ₁-heterocyclic ring, wherein m1 is 0 to 2;
   L₂ is absent or (1-2C)alkylene;
   R_{C} is selected from hydrogen, (1-4C)alkyl, (3-6C)cycloalkyl, phenyl, heteroaryl or heterocyclyl;
   wherein:
      the alkyl, cycloalkyl, phenyl, is substituted by one or more R_{A} substituents and is optionally further substituted by one or more R_{B} substituents;
      if the heteroaryl or heterocyclic ring does not comprise one or more N atoms having a pKa of 5.5 to 9, or 6 to 9, or 6.2 to 8.6, then the ring is substituted by one or more R_{A} substituents and is optionally substituted by one or more R_{B} substituents, or if the heteroaryl or heterocyclic ring does comprise one or more N atoms having a pKa of 5.5 to 9, or 6 to 9, or 6.2 to 8.6, then the ring is optionally substituted by one or more R_{A} or R_{B} substituents;
   R_{A} is a group selected from:
      (i) —OH;
      (ii) NR_{A1}R_{A2};
      (iii) -(1-6C)alkylene-NR_{A1}R_{A2};
      wherein:
         R_{A1} and R_{A2} are each independently selected from hydrogen, (1-6C)alkyl, or (1-6C)heteroalkyl;
   R_{B} is selected from halo, nitro, cyano, R_{BA}, —[CH₂]ₜ—OR_{BA}, —[CH₂]ₜ—C(O)R_{BA}, —[CH₂]ₜ—C(O)OR_{BA}, —[CH₂]ₜ—OC(O)R_{BA}, —[CH₂]ₜ—C(O)N(R_{BB})R_{BA}, —[CH₂]ₜ—N(R_{BB})C(O)R_{BA}, —[CH₂]ₜ—S(O)ₚR_{BA} (where p is 0, 1 or 2), —[CH₂]ₜ—SO₂N(R_{Bb})R_{BA}, or —[CH₂]ₜ—N(R_{BB})SO₂R_{BA};
      wherein t is 0, 1, 2 or 3;
      R_{BA} is hydrogen or (1-4C)alkyl optionally substituted by halo, hydroxy, amino or cyano; and
      R_{BB} is hydrogen or (1-2C)alkyl;
wherein the cleavage group is not:
   a) an imidazole (1,3-diazole) group that is optionally substituted with one, two or three (1-6C)alkyl groups, which may be the same or different; or
   b) a group of the formula Z defined above;
(7) the cleavage group C is a group of the formula:

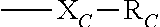

wherein:
   X_{C} is absent or selected from —O—, —S—, —SO—, —SO₂—, —N(R_{XC1})—, —C(O)—, —C(O)N(R_{XC1})— or —N(R_{XC1})C(O)—; and wherein R_{XC1} is selected from hydrogen or (1-6C)alkyl or —(CH₂)ₘ₁-heteroaryl, wherein m1 is 0 to 2;
   R_{C} is selected from hydrogen, (1-4C)alkyl, phenyl, heteroaryl or heterocyclyl;
   wherein:
      the alkyl, cycloalkyl, phenyl, is substituted by one or more R_{A} substituents and is optionally further substituted by one or more R_{B} substituents;
      if the heteroaryl or heterocyclic ring does not comprise one or more N atoms having a pKa of 5.5 to 9, or 6 to 9, or 6.2 to 8.6, then the ring is substituted by one or more R_{A} substituents and is optionally substituted by one or more R_{B} substituents, or if the heteroaryl or heterocyclic ring does comprise one or more N atoms having a pKa of 5.5 to 9, or 6 to 9, or 6.2 to 8.6, then the ring is optionally substituted by one or more R_{A} or R_{B} substituents;
   R_{A} is a group selected from:
      (i) —OH;
      (ii) NR_{A1}R_{A2},
      (iii) -(1-6C)alkylene-NR_{A1}R_{A2};
      wherein:
         R_{A1} and R_{A2} are each independently selected from hydrogen, (1-6C)alkyl, or (1-6C)heteroalkyl;
   R_{B} is selected from halo, nitro, cyano, R_{BA}, —[CH₂]ₜ—OR_{BA} or —[CH₂]ₜ—C(O)OR_{BA}, wherein t is 0, 1, 2 or 3; and
      R_{BA} is (1-4C)alkyl;
wherein the cleavage group is not:
   a) an imidazole (1,3-diazole) group that is optionally substituted with one, two or three (1-6C)alkyl groups, which may be the same or different; or
   b) a group of the formula Z defined above;
(8) the cleavage group C is selected from a group of the formula:

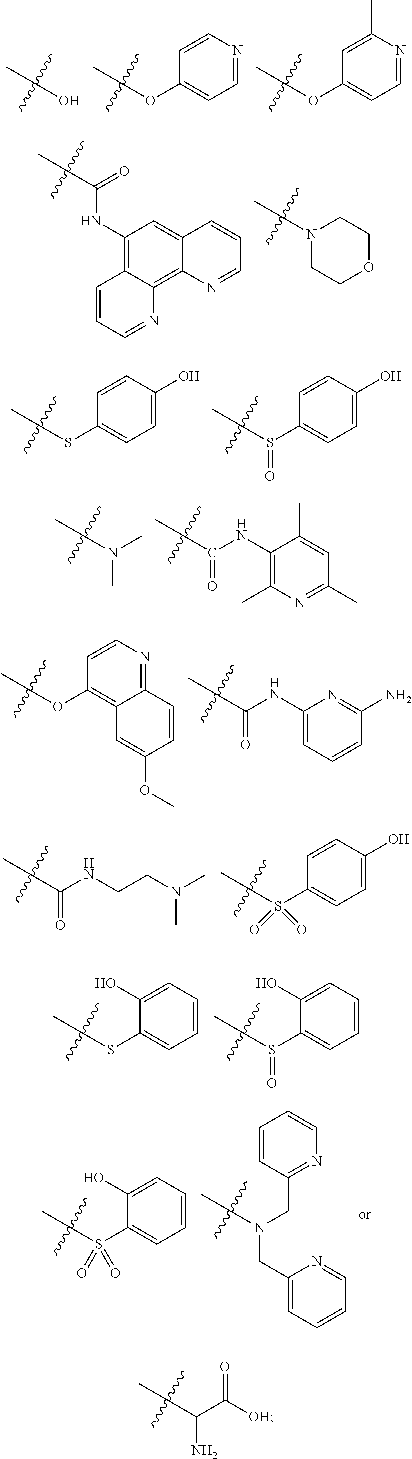

(9) the cleavage group C is selected from a group of the formula:

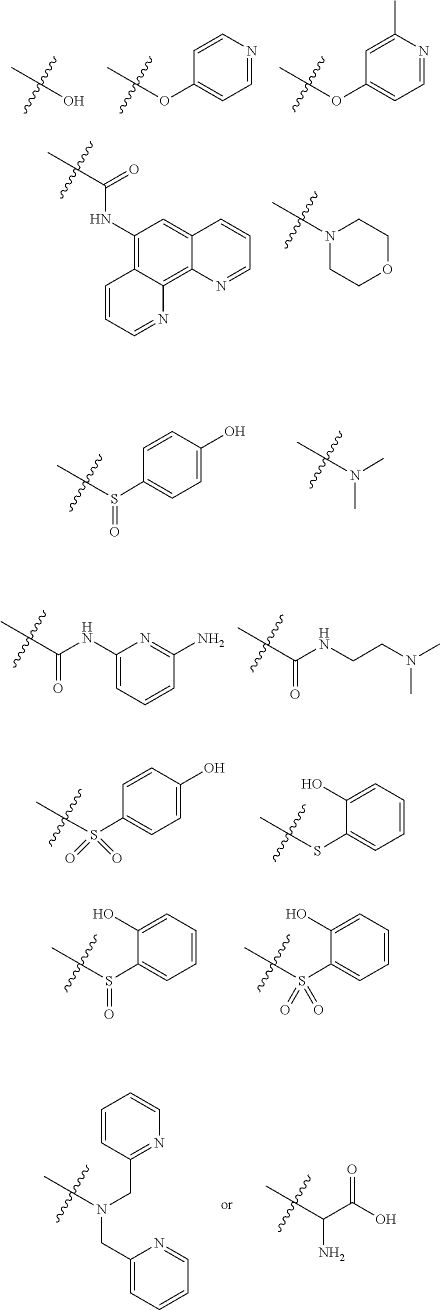

(10) the cleavage group C is selected from a group of the formula:

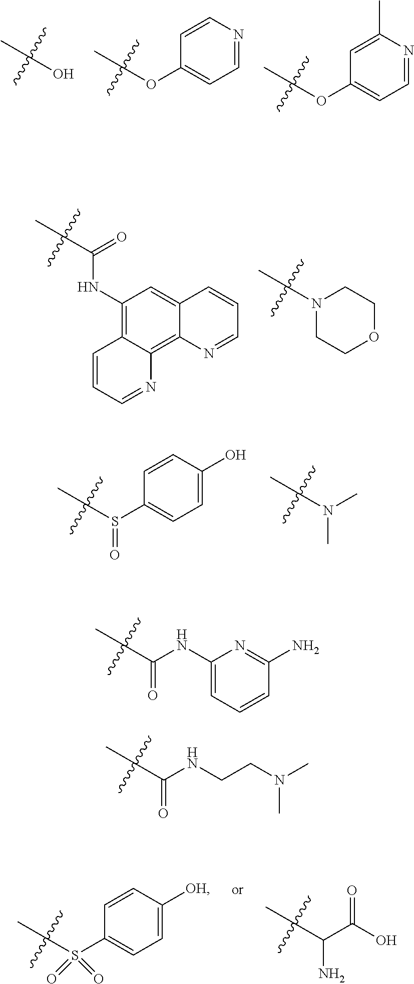

(11) the cleavage group C is selected from a group of the formula:

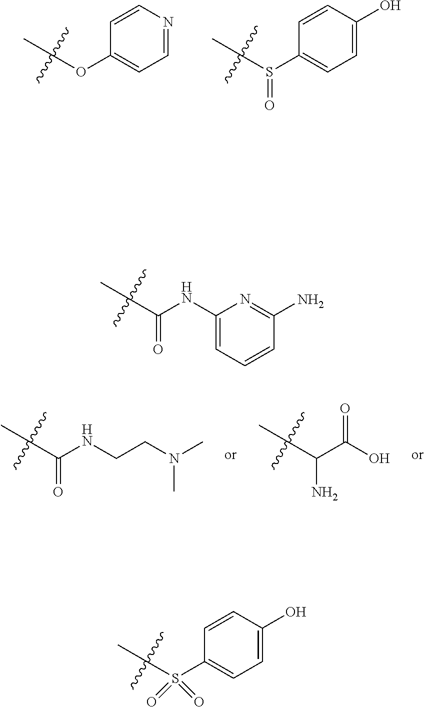

(12) the cleavage group C is a group of the formula:

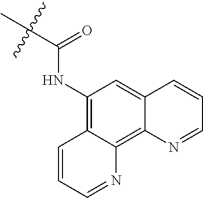 [SMILES: *C(=O)Nc1cc2cccnc2c2ncccc12]

In an embodiment of the invention, the cleavage group C is as defined in paragraph (1) above.

In an embodiment of the invention, the cleavage group C is as defined in paragraph (2) above. In an embodiment of the invention, the cleavage group C is as defined in paragraph (2A) above. In an embodiment of the invention, the cleavage group C is as defined in paragraph (3) above. In an embodiment of the invention, the cleavage group C is as defined in paragraph (3A) above. In an embodiment of the invention, the cleavage group C is as defined in paragraph (4) above. In an embodiment of the invention, the cleavage group C is as defined in paragraph (4A) above. In an embodiment of the invention, the cleavage group C is as defined in paragraph (5) above. In an embodiment of the invention, the cleavage group C is as defined in paragraph (6) above. In an embodiment of the invention, the cleavage group C is as defined in paragraph (7) above. In an embodiment of the invention, the cleavage group C is as defined in paragraph (8) above. In an embodiment of the invention, the cleavage group C is as defined in paragraph (9) above. In an embodiment of the invention, the cleavage group C is as defined in paragraph (10) above. In an embodiment of the invention, the cleavage group C is as defined in paragraph (11) above. In an embodiment of the invention, the cleavage group C is as defined in paragraph (12) above.

As indicated above, when non-covalently bound to the target nucleic acid molecule through the linker and binding group, the cleavage group C is proximate to the target nucleic acid and reacts with the target nucleic acid molecule to cleave one or more phosphodiester bonds, thereby causing degradation of the target nucleic acid molecule. For example, the cleavage group C of the bound degrader may abstract a proton from the 2′OH position on the nucleic acid molecule leading to cleavage of a phosphodiester bond in the target nucleic acid molecule. In addition, the cleavage group C may form a copper complex which cleaves a phosphodiester bond in the target nucleic acid molecule.

Suitably, one of R_{C} or a R_{A} substituent group comprises a basic nitrogen atom or hydroxy group that either: (i) has a pKa within the range of 5.5 to 9, or 6 to 9, or 6.2 to 8.6; and/or (ii) is a nitrogen atom or hydroxy group that is capable of forming chelation complex with copper (and is thereby capable of inducing copper-mediated nucleic acid degradation).

Suitably, when R_{C} is alkyl, cycloalkyl, or aryl/phenyl, then R_{A} is a substituent group comprising a basic nitrogen atom or hydroxy group that either: (i) has a pKa within the range of 5.5 to 9, or 6 to 9, or 6.2 to 8.6; and/or (ii) is a nitrogen atom or hydroxy group that is capable of forming chelation complex with copper (and is thereby capable of inducing copper-mediated nucleic acid degradation).

Suitably, when R_{C} is heteroaryl or heterocyclyl, then either the heteroaryl or heterocyclyl group comprises a basic nitrogen atom that either: (i) has a pKa within the range of 5.5 to 9, or 6 to 9, or 6.2 to 8.6; and/or (ii) is a nitrogen atom that is capable of forming chelation complex with copper (and is thereby capable of inducing copper-mediated nucleic acid degradation), or the heteroaryl or heterocyclyl is substituted by a R_{A} substituent group comprising a basic nitrogen atom or hydroxy group that either: (i) has a pKa within the range of 5.5 to 9, or 6 to 9, or 6.2 to 8.6; and/or (ii) is a nitrogen atom or hydroxy group that is capable of forming chelation complex with copper (and is thereby capable of inducing copper-mediated nucleic acid degradation).

### Linker (-L-)

The linker L of the degrader comprises a group for connection (i.e. covalent connection) of the cleavage group (C) to the non-covalent binding group (B). Suitable linkers are well known in the art.

Typically, the linker comprises a divalent group in which one of the free valencies forms part of a single bond to the cleavage group (C) and the remaining free valency forms part of a single bond to the non-covalent binding group (B).

Suitably, the linker is a stable linker. That is, the linker comprises a group that is not substantially cleaved or degraded in vivo. A stable linker is typically unreactive at physiological pH, and not substantially degraded by enzymatic action in vivo.

Typically, the linker is a flexible linker. That is, the linker permits the cleavage group (C) and binding group (B) to move relative to each other with a large degree of freedom.

Typical linkers comprise groups selected from alkylene, heteroalkylene, cycloalkylene, heterocycloalkylene, arylene and heteroarylene. Mixed linkers comprising different groups in covalent connection, such as alkylene-arylene (aralkylene) and heteroalkylene-arylene, may be permitted.

An alkylene (alkanediyl) group is a divalent saturated hydrocarbon group in which the two free valencies each form part of a single bond to an adjacent atom. The alkylene group may be a (1-6C)alkylene group, for example, a 1-4C, 1-3C or a 1-2C alkylene group. In this context, the prefix (e.g. 1-6C) denotes the number of atoms in the hydrocarbon backbone. The alkylene group may be linear or branched. Examples of linear alkylene groups include methanediyl (methylene bridge), ethane-1,2-diyl (ethylene bridge), propane-1,3-diyl, butan-1,4-diyl, pentan-1,5-diyl and hexan-1,6-diyl. Examples of branched alkylene groups include ethane-1,1-diyl and propane-1,2-diyl.

A heteroalkylene group is an alkylene group in which one or more carbon atoms is replaced with a heteroatom, for example N, O and S. The heteroalkylene group may be a 1-6C heteroalkylene group, for example, a 1-4C, 1-3C or a 1-2C heteroalkylene group. In this context, the prefix (e.g. 1-6C) denotes the number of atoms in the heteroalkylene backbone, whether carbon atoms or heteroatoms. The heteroalkylene group may be linear or branched.

Examples of linear heteroalkylene groups include those derived from oxymethylene (e.g. polyoxymethylene, POM), ethylene glycol (e.g. polyethylene glycol, PEG), ethylenimine (e.g. linear polyethylenimine, PEI; polyaziridine) and tetramethylene glycol (e.g. polytetramethylene glycol, PTMEG; polytetrahydrofuran). Examples of branched heteroalkylene groups include those derived from propylene glycol (e.g. polypropylene glycol PPG). Where a nitrogen atom is present in a heteroalkylene group, that nitrogen atom may be unsubstituted (NH) or optionally substituted with an alkyl group, such as a (1-4C)alkyl group. Where a sulfur atom is present in a heteroalkyl group, that sulfur atom may be S, S(O) or S(O)₂.

A cycloalkylene group is a divalent saturated hydrocarbon group which comprises a ring in which all of the ring atoms are carbon atoms, and in which the two free valencies each form part of a single bond to an adjacent atom. The cycloalkylene group may be a (5-6C)cycloalkylene group. In this context, the prefix (e.g. 5-6C) denotes the number or range of ring atoms. The cycloalkylene group may be monocyclic. Examples of monocylic cycloalkylene groups include 1,3-cyclopentylene and 1,4-cyclohexylene.

A heterocycloalkylene (heterocyclene) group is a cycloalkylene group in which one or more carbon atoms is replaced with a heteroatom, for example N, O and S, or in which one or more carbon atoms has an oxo substituent (═O). The heterocycloalkylene group may be a C5-6heterocycloalkylene group. In this context, the prefix (e.g. 5-6C) denotes the number or range of ring atoms, whether carbon atoms or heteroatoms. The heterocycloalkylene group may be monocyclic. Where a nitrogen atom is present in a heteroalkylene group, that nitrogen atom may be unsubstituted (NH) or optionally substituted with an alkyl group, such as a 1-4C alkyl group. Where a sulfur atom is present in a heteroalkyl group, that sulfur atom may be S, S(O) or S(O)₂.

An arylene group is a divalent hydrocarbon group comprising an aromatic ring in which all of the ring atoms are carbon atoms, and in which the two free valencies each form part of a single bond to an adjacent atom. The arylene group may be a 6-10C arylene group. In this context, the prefix (e.g. 6-10C) denotes the number or range of ring atoms. The arylene group may be monocyclic, or it may comprise two or more rings. Examples of monocyclic arylene groups include 1,4-phenylene. Examples of bicyclic arylene groups include 2,6-naphthylene.

A heteroarylene group is an arylene group comprising an aromatic ring in which one or more ring atoms are heteroatoms, for example N, O and S, or in which one or more carbon atoms has an oxo substituent (═O). The heteroarylene group may be a 6-10C heteroarylene group. In this context, the prefix (e.g. 6-10C) denotes the number or range of ring atoms, whether carbon or heteroatom. The heteroarylene group may be monocyclic, or it may comprise two or more rings. Examples of monocyclic heteroarylene groups include pyrrolylene and pyridylene.

Suitable linkers comprise groups selected from alkylene and heteroalkylene. More suitable linkers comprise heteroalkylene groups. Even more suitable linkers comprise alkylene ether groups. The most suitable linkers comprise ethylene oxide groups (e.g. derived from polyethylene glycol, PEG).

Particular compounds of the invention include, for example, compounds of Formula (I) or any sub-formula thereof, or pharmaceutically acceptable salts and/or solvates thereof, wherein, unless otherwise stated, the linker group L, and any associated substituent groups has any of the meanings defined hereinbefore or in any of paragraphs (13) to (34) hereinafter:—
(13) the linker is or comprises a group represented by formula (L-Ia) or (L-Ib):

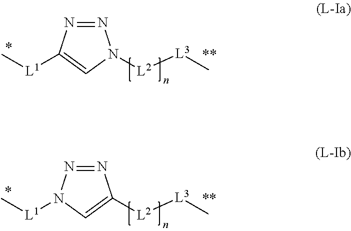

wherein:
   L¹ is a covalent bond or a (1-6C)alkylene group or (1-6C)heteroalkylene;
   L² is a (1-6C)alkylene group or a (1-6C)heteroalkylene group;
   L³ is a (1-6C)alkylene group;
   n is 0 to 8;
   * is the attachment point with the non-covalent binding group (—B); and
   ** is the attachment point with the cleavage group (—C).
(14) The linker defined in paragraph (13) wherein L¹ is a covalent bond or methylene;
(15) The linker defined in paragraph (13) or (14) wherein L³ is (1-4C)alkylene;
(16) The linker defined in paragraph (13) or (15) wherein L³ is ethylene;
(17) The linker defined in paragraphs (13) to (16) wherein n is 0 to 5 or 2 to 5;
(18) The linker defined in paragraphs (13) to (17) wherein L² is selected from —CH₂—X—, —CH₂—CH₂—X—, —CH₂—CH₂—CH₂—X—, or —CH₂—CH₂—CH₂—CH₂—X—, wherein X is —O— or —NH—;
(19) The linker defined in paragraphs (13) to (18) wherein L² is selected from —CH₂—X—, —CH₂—CH₂—X—, or —CH₂—CH₂—CH₂—X—, wherein X is —O— or —NH—;
(20) The linker defined in paragraphs (13) to (19) wherein L² is selected from —CH₂—X— or —CH₂—CH₂—X—, wherein X is —O— or —NH—;
(21) The linker defined in paragraphs (13) to (20) wherein L² is selected from —CH₂—O—, —CH₂—NH—, ethylene oxide (—CH₂CH₂O—), propylene oxide (—CH₂CH₂CH₂O—) and tetramethylene oxide (—CH₂CH₂CH₂CH₂O—);
(22) The linker defined in paragraphs (13) to (21) wherein L² is ethylene oxide; (—CH₂CH₂O—);
(23) The linker is or comprises a group represented by formula (L-IIa) or (L-IIb):

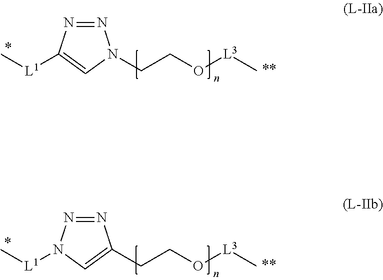

wherein L¹, L³, n, * and ** are as described for formula (L-1) in paragraph (13) above, and L¹ is optionally as defined in paragraph (14), L³ is optionally as defined in paragraph (15) or (16) and n is optionally as defined in paragraph (17).
(24) the linker is or comprises a group represented by formula (L-IIIa) or (L-IIIb):

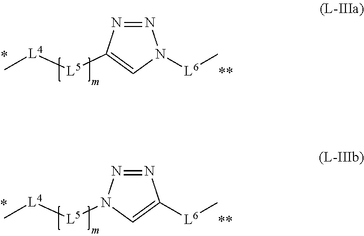

wherein:
   L⁴ is a (1-6C)alkylene group;
   L⁵ is a (1-6C)alkylene group or a (1-6C)heteroalkylene group;
   L⁶ is a covalent bond or a (1-2C)alkylene group;
   m is 1 to 8;
   * is the attachment point with the non-covalent binding group (—B); and
   ** is the attachment point with the cleavage group (—C).
(25) the linker defined in paragraph (24) wherein L⁰ is a covalent bond or methylene;
(26) the linker defined in paragraph (24) or (25) wherein L⁴ is (1-4C)alkylene;
(27) the linker defined in paragraph (24) to (26) wherein L⁴ is ethylene;
(28) the linker defined in paragraphs (24) to (27) wherein m is 0 to 5 or 2 to 5;
(29) the linker defined in paragraphs (24) to (28) wherein L⁵ is selected from —CH₂—X—, —CH₂—CH₂—X—, —CH₂—CH₂—CH₂—X—, or —CH₂—CH₂—CH₂—CH₂—X—, wherein X is —O— or —NH—;
(30) The linker defined in paragraphs (24) to (29) wherein L⁵ is selected from —CH₂—X—, —CH₂—CH₂—X—, or —CH₂—CH₂—CH₂—X—, wherein X is —O— or —NH—;
(31) The linker defined in paragraphs (24) to (30) wherein L⁵ is selected from —CH₂—X— or —CH₂—CH₂—X—, wherein X is —O— or —NH—;
(32) The linker defined in paragraphs (24) to (31) wherein L⁵ is selected from —CH₂—O—, —CH₂—NH—, ethylene oxide (—CH₂CH₂O—), propylene oxide (—CH₂CH₂CH₂O—) and tetramethylene oxide (—CH₂CH₂CH₂CH₂O—);
(33) The linker defined in paragraphs (24) to (32) wherein L⁵ is ethylene oxide (—CH₂CH₂O—).
(34) the linker is or comprises a group represented by formula (L-IV) or (L-V):

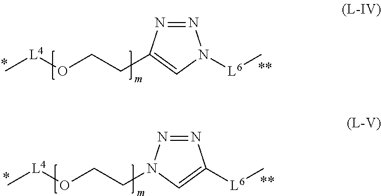

wherein L⁴, L⁶, m, * and ** are as described for formula (L-III) in paragraph (24) above, and L⁴ is optionally as defined in paragraph (26) or (27), L⁶ is optionally as defined in paragraph (25) and m is optionally as defined in paragraph (28).

Further suitable linkers are of the formula:

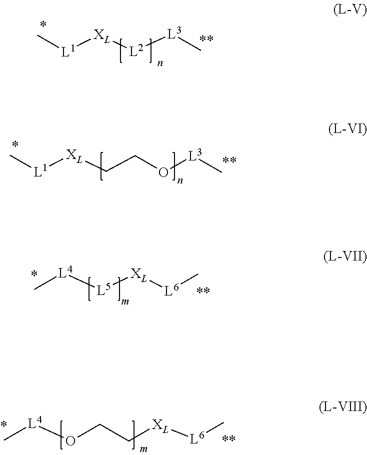

wherein:
L¹, L², L³, n, *, **, L⁴, L⁵, L⁶, and m are each as defined above; and
X_{L} is selected from —O—, —S—, —SO—, —SO₂—, —NH—, —C(O)—, —C(O)O—, —OC(O)—, —C(O)NH— or —NHC(O)—, piperidine, piperazine or triazole.

Suitably, L¹ is as defined in paragraph (14) above.

Suitably, L³ is as defined in paragraph (15) or (16) above.

Suitably, n is as defined in paragraph (17) above.

Suitably, L² is as defined in any one of paragraph (18) to (22) above.

Suitably, L¹ is as defined in paragraph (14) above, L² is as defined in paragraph (18) above, L³ is as defined in paragraph (15) above and n is as defined in paragraph (17) above.

Suitably, L¹ is as defined in paragraph (14) above, L² is as defined in paragraph (19) above, L³ is as defined in paragraph (16) above and n is as defined in paragraph (17) above.

Suitably, L¹ is as defined in paragraph (14) above, L² is as defined in paragraph (20) above, L³ is as defined in paragraph (16) above and n is as defined in paragraph (17) above.

Suitably, L¹ is as defined in paragraph (14) above, L² is as defined in paragraph (21) above, L³ is as defined in paragraph (16) above and n is as defined in paragraph (17) above.

Suitably, L¹ is as defined in paragraph (14) above, L² is as defined in paragraph (22) above, L³ is as defined in paragraph (16) above and n is as defined in paragraph (17) above.

Suitably, L⁶ is as defined in paragraph (25) above.

Suitably, L⁴ is as defined in paragraph (26) or (27) above.

Suitably, m is as defined in paragraph (28) above.

Suitably, L⁵ is as defined in any one of paragraphs (26) or (27) above.

Suitably, L⁶ is as defined in paragraph (22) above, L⁴ is as defined in paragraph (24) above, L⁵ is as defined in paragraph (26) above and m is as defined in paragraph (25) above.

Suitably, L⁶ is as defined in paragraph (22) above, L⁴ is as defined in paragraph (24) above, L⁵ is as defined in paragraph (27) above and m is as defined in paragraph (25) above.

Suitable (1-2C)alkylene groups include methylene (methanediyl), ethylene (ethane-1,2-diyl).

Suitable (1-6C)alkylene groups include methylene (methanediyl), ethylene (ethane-1,2-diyl), propylene (propane-1,3-diyl), butylene (butan-1,4-diyl), pentylene (pentan-1,5-diyl) and hexylene (hexan-1,6-diyl).

Suitable (1-6C)heteroalkylene groups include alkylene ether group such as ethylene oxide (—CH₂CH₂O—), propylene oxide (—CH₂CH₂CH₂O—) and tetramethylene oxide (—CH₂CH₂CH₂CH₂O—).

Suitably L² is ethylene oxide. Suitably L⁴ is (1-4C)alkylene. Most suitably L³ is ethylene.

Suitably L⁶ is methylene or ethylene. Suitably m is 2 to 5. Suitably L⁵ is ethylene oxide.

In an embodiment, m or n is 4 to 8, 5 to 7, or 6.

### Non-Covalent Binding Group (—B)

The binding group of the degrader comprises a group capable of binding to the target nucleic acid molecule. The binding group binds to the target nucleic acid molecule through non-covalent bonding.

Certain small-molecule ligands are known to non-covalently bind to nucleic acids, and thus may form the basis of the non-covalent binding group. Any small molecule capable of binding to nucleic acids may be used as the non-covalent binding group. Such compounds may, for example, bind to MYC or MALAT-1. Suitably, the small molecule compounds are capable of binding to a secondary or tertiary structure within the target nucleic acid. In an embodiment, the non-covalent binding group may target SNVs and indels that get transcribed (for example, rs4430796 SNP on HNF1B is associated with ovarian and prostate cancers; rs28897672 SNV on BRCA1 is associated with ovarian cancer; rs80359351 deletion on BRCA2 is associated with breast and ovarian cancers—all of these genetic changes are reflected in respective mRNAs thus are potentially targetable with the degraders of the present invention).

In addition, the non-colvalent binding group B may be an oligonucleotide, nanobody, antibody or antibody fragment that is capable of binding to a target nucleic acid secquence. The binding of the oligonucleotide binding group B to target nuceic acid sequence, enables the targeted cleavage/degradation of the target nucleic acid molecule. The target nucleic acid sequence may be any desired nucleic acid sequence, including sequences associated with particular medical conditions, e.g. sequences associated with cancer, nucleotide repeat disorders (e.g. Huntington's, Fragile X, Myotonic Dystrophy Type 1), and sequences of mRNAs encoding non-structured proteins (IAPP in Type II Diabetes).

In some embodiments, the non-covalent binding group has molecular weight of 1,000 kDa or less. For example, the non-covalent binding group has a molecular weight of 800 kDa or less.

In certain embodiments, the non-covalent binding group binds to a secondary or tertiary structure within the target nucleic acid. Suitable secondary or tertiary structures include quadruplexes, pseudoknots, triplexes, tetraloops, step-loops and hairpin loops. Suitably, the non-covalent binding group binds to a quadruplex or pseudoknot.

Suitably, in such embodiments, the non-covalent binding group selectively binds to a secondary or tertiary structure within the target nucleic acid. In such cases, the non-covalent binding group preferentially binds to a secondary or tertiary structure within the target nucleic acid in comparison to linear or unstructured nucleic acid. Suitably, the non-covalent binding group selectively binds to a quadruplex or pseudoknot.

Suitably, the non-covalent binding group selectively binds to a ribonucleic acid (RNA). Accordingly, the non-covalent binding group may be known as a non-covalent RNA binding group.

The non-covalent binding group may bind to the target nucleic acid through electrostatic interactions, such as ionic interactions, hydrogen-bonding and halogen bonding; van der Waals interactions such as permanent dipole-dipole interactions, dipole-induced dipole interactions, and induced dipole-induced dipole interactions; and π-effects such as π-πinteractions, π-cation interactions and polar-π interactions.

The non-covalent binding group may be based on the following small molecule nucleic acid binding molecules:

| Name | Structure | Binding group |
|---|---|---|
| Pyridostatin (PDS) | 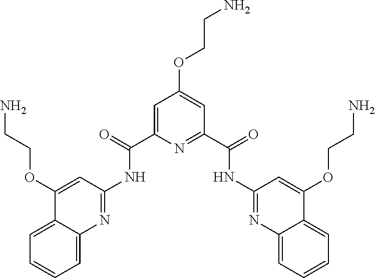 | 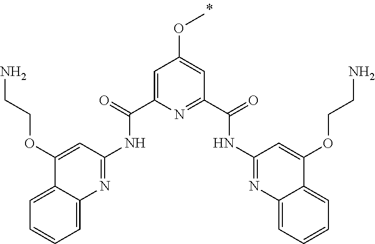 (B-I) |
| MTBD | 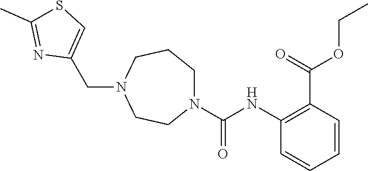 | 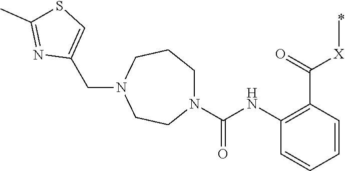 X is O or NH (B-II) |
| Chloramphenicol | 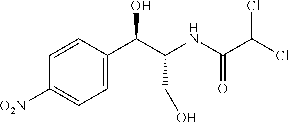 | 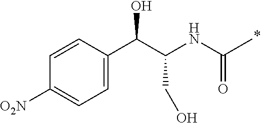 (B-III) |
| Lincomycin | 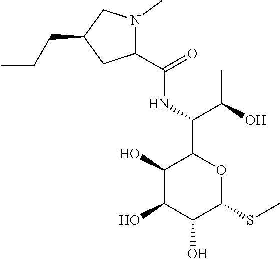 | 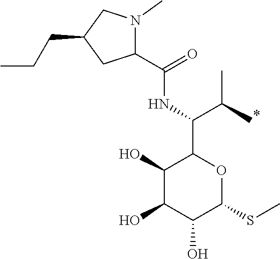 (B-IV) |

*is the attachment point with the linker

The non-covalent binding group may be attached to the linker at any suitable position.

Typically, the non-covalent binding group is attached to the linker through a heteroatom (such as 0 or NH), or adjacent to a carbonyl group (0=0).

Suitably, the binding group is selected from formulae (B—I), (B-II), (B—III) or (B—IV).

Particular compounds of the invention include, for example, compounds of Formula (I) or any sub-formula thereof, or pharmaceutically acceptable salts and/or solvates thereof, wherein, unless otherwise stated, the non-covalent binding group B, and any associated substituent groups has any of the meanings defined hereinbefore or in any of paragraphs (35) to (39) hereinafter:—
(35) the binding group B is selected from an oligonucleotide, nanobody, antibody, antibody fragment or small molecule capable of binding a target nucleic acid or one of formulae (B-1), (B-II), (B-Ill) or (B—IV) above;
(36) the binding group B is a group of formula (B-1) above;
(37) the binding group B is a group of formula (B-II) above;
(38) the binding group B is a group of formula (B—III) above;
(39) the binding group B is a group of formula (B—IV) above.

### Particular Embodiments

In a particular embodiment:
C is as defined in paragraph (1) above;
L is as defined in any one of paragraphs (13) to (34) above; and
B is as defined in paragraph (35) above.

In a particular embodiment:
C is as defined in paragraph (2) above;
L is as defined in any one of paragraphs (13) to (34) above; and
B is as defined in paragraph (35) above.

In a particular embodiment:
C is as defined in paragraph (2A) above;
L is as defined in any one of paragraphs (13) to (34) above; and
B is as defined in paragraph (35) above.

In a particular embodiment:
C is as defined in paragraph (3) above;
L is as defined in any one of paragraphs (13) to (34) above; and
B is as defined in paragraph (35) above.

In a particular embodiment:
C is as defined in paragraph (3A) above;
L is as defined in any one of paragraphs (13) to (34) above; and
B is as defined in paragraph (35) above.

In a particular embodiment:
C is as defined in paragraph (4) above;
L is as defined in any one of paragraphs (13) to (34) above; and
B is as defined in paragraph (35) above.

In a particular embodiment:
C is as defined in paragraph (4A) above;
L is as defined in any one of paragraphs (13) to (34) above; and
B is as defined in paragraph (35) above.

In a particular embodiment:
C is as defined in paragraph (5) above;
L is as defined in any one of paragraphs (13) to (34) above; and
B is as defined in paragraph (35) above.

In a particular embodiment:
C is as defined in paragraph (6) above;
L is as defined in any one of paragraphs (13) to (34) above; and
B is as defined in paragraph (35) above.

In a particular embodiment:
C is as defined in paragraph (7) above;
L is as defined in any one of paragraphs (13) to (34) above; and
B is as defined in paragraph (35) above.

In a particular embodiment:
C is as defined in paragraph (8) above;
L is as defined in any one of paragraphs (13) to (34) above; and
B is as defined in paragraph (35) above.

In a particular embodiment:
C is as defined in paragraph (9) above;
L is as defined in any one of paragraphs (13) to (34) above; and
B is as defined in paragraph (35) above.

In a particular embodiment:
C is as defined in paragraph (10) above;
L is as defined in any one of paragraphs (13) to (34) above; and
B is as defined in paragraph (35) above.

In a particular embodiment:
C is as defined in paragraph (11) above;
L is as defined in any one of paragraphs (13) to (34) above; and
B is as defined in paragraph (35) above.

In a particular embodiment:
C is as defined in paragraph (12) above;
L is as defined in any one of paragraphs (13) to (34) above; and
B is as defined in paragraph (35) above.

In a particular embodiment, B is a pyridostatin binding group, i.e. the compounds have the formula (II) shown below:

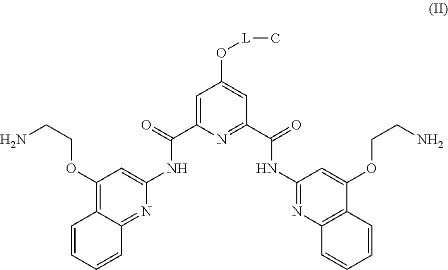

wherein L and C are each as defined above.

In a particular embodiment, B is a MTBD binding group, i.e. the compounds have the formula (III) shown below:

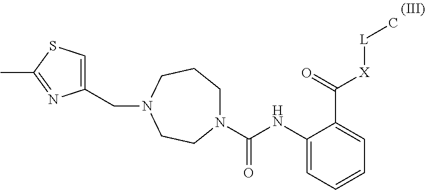

wherein L, X and C are each as defined above.

In a particular embodiment, B is a chloramphenicol binding group, i.e. the compounds have the formula (IV) shown below:

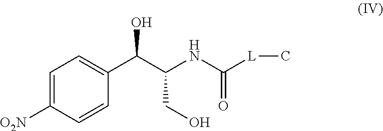

wherein L and C are each as defined above.

In a particular embodiment, B is a lincomycin binding group, i.e. the compounds have the formula (V) shown below:

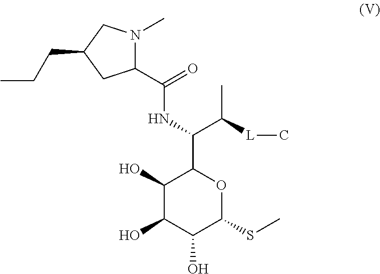

wherein L and C are each as defined above.

Suitably, in compounds of formula (II), (III), (IV) or (V):
the cleavage group C is as defined in any one of paragraphs (1) to (12) above (including 2A, 3A and 4A); and
L is as defined in any one of paragraphs (13) to (34) above.

In particular compounds of formula (II), (Ill), (IV) or (V):
the cleavage group C is as defined in paragraph (1) above; and
L is as defined in any one of paragraphs (13) to (34) above.

In particular compounds of formula (II), (III), (IV) or (V):
the cleavage group C is as defined in paragraph (2) above; and
L is as defined in any one of paragraphs (13) to (34) above.

In particular compounds of formula (II), (III), (IV) or (V):
the cleavage group C is as defined in paragraph (2A) above; and
L is as defined in any one of paragraphs (13) to (34) above.

In particular compounds of formula (II), (Ill), (IV) or (V):
the cleavage group C is as defined in paragraph (3) above; and
L is as defined in any one of paragraphs (13) to (34) above.

In particular compounds of formula (II), (III), (IV) or (V):
the cleavage group C is as defined in paragraph (4A) above; and
L is as defined in any one of paragraphs (13) to (34) above.

In particular compounds of formula (II), (III), (IV) or (V):
the cleavage group C is as defined in paragraph (4) above; and
L is as defined in any one of paragraphs (13) to (34) above.

In particular compounds of formula (II), (Ill), (IV) or (V):
the cleavage group C is as defined in paragraph (4A) above; and
L is as defined in any one of paragraphs (13) to (34) above

In particular compounds of formula (II), (III), (IV) or (V):
the cleavage group C is as defined in paragraph (5) above; and
L is as defined in any one of paragraphs (13) to (34) above.

In particular compounds of formula (II), (III), (IV) or (V):
the cleavage group C is as defined in paragraph (6) above; and
L is as defined in any one of paragraphs (13) to (34) above.

In particular compounds of formula (II), (Ill), (IV) or (V):
the cleavage group C is as defined in paragraph (7) above; and
L is as defined in any one of paragraphs (13) to (34) above.

In particular compounds of formula (II), (III), (IV) or (V):
the cleavage group C is as defined in paragraph (8) above; and
L is as defined in any one of paragraphs (13) to (34) above.

In particular compounds of formula (II), (III), (IV) or (V):
the cleavage group C is as defined in paragraph (9) above; and
L is as defined in any one of paragraphs (13) to (34) above.

In particular compounds of formula (II), (Ill), (IV) or (V):
the cleavage group C is as defined in paragraph (10) above; and
L is as defined in any one of paragraphs (13) to (34) above.

In particular compounds of formula (II), (III), (IV) or (V):
the cleavage group C is as defined in paragraph (11) above; and
L is as defined in any one of paragraphs (13) to (34) above.

In particular compounds of formula (II), (III), (IV) or (V):
the cleavage group C is as defined in paragraph (12) above; and
L is as defined in any one of paragraphs (13) to (34) above.

Particular compounds of the invention include compounds of formula VI shown below, or a pharmaceutically acceptable salt thereof:

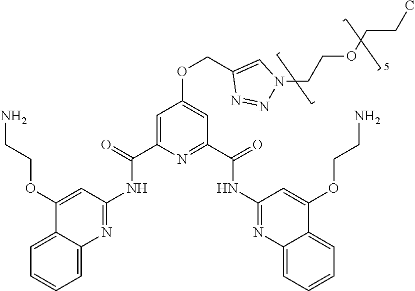

wherein C is as defined herein before, or selected from one of the following:

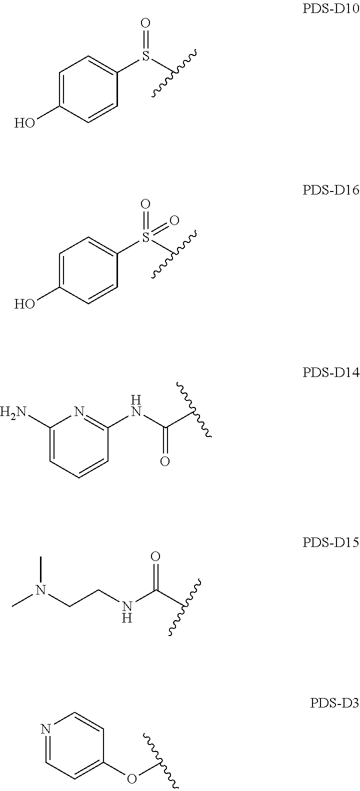

### Kinetic Properties

The interaction between the degrader and the target nucleic acid can be quantified using the dissociation constant (kD). The dissociation constant between a degrader comprising a given non-covalent binding group and a nucleic acid may be known or it may be determined using standard techniques such as surface plasmon resonance (SPR), for example Biacore (Santos, et al., 2021). Suitable systems for measuring the dissociation constant include Biacore T200.

Typically, the degrader binds to the target nucleic acid with a dissociation constant (kD) of 10,000 nM or less, such as determined by SPR. Suitably, the degrader binds to the target nucleic acid with a kD of 1,000 nM or less, more suitably 500 nM or less, even more preferably 200 nM or less, and most suitably 100 nM or less.

As noted above, the non-covalent binding group of degrader typically binds to a secondary or tertiary structure within the target nucleic acid. Accordingly, the degrader typically binds to a secondary or tertiary structure with a dissociation constant (kD) of 10,000 nM or less, such as determined by SPR. Suitably, the degrader binds to the secondary or tertiary structure with a kD of 1,000 nM or less, more suitably 500 nM or less, even more suitably 200 nM or less, and most suitably 100 nM or less.

In some embodiments, the degrader binds to a quadruplex with a dissociation constant (kD) of 10,000 nM or less, such as determined by SPR. In such cases, the degrader suitably binds to the quadruplex with a kD of 1,000 nM or less, more suitably 500 nM or less, even more suitably 200 nM or less, and most suitably 100 nM or less.

In some embodiments, the degrader binds to a pseudoknot with a dissociation constant (kD) of 10,000 nM or less, such as determined by SPR. In such cases, the degrader suitably binds to the pseudoknot with a kD of 1,000 nM or less, more suitably 500 nM or less, even more suitably 200 nM or less, and most suitably 100 nM or less.

As noted above, the non-covalent binding group of the degrader suitably selectively binds to a secondary or tertiary structure within the target nucleic acid. The binding selectivity can be quantified using the ratio between the dissociation constant for binding to a given secondary or tertiary structure in compression to the dissociation constant for binding to linear or unstructured nucleic acid, such as linear or unstructured RNA. Typically, the comparison linear or unstructured nucleic acid is prepared by mutating one or more residues within the secondary or tertiary structure of interest such that the secondary or tertiary structure no longer forms, while the remainder of the sequence is maintained. For example, the selectivity of binding to an RNA G quadruplex can be assessed by using comparison RNA in which one or more GGG motifs are exchanged for AUC motifs.

Typically, the binding selectivity between a given secondary or tertiary structure and linear or unstructured nucleic acid is 5:1 or greater. Suitably, selectivity between a given secondary or tertiary structure and linear or unstructured nucleic acid is 10:1 or greater, more suitably 20:1 or greater, even more suitably 50:1 or greater, and most suitably 100:1 or greater.

In one embodiment, the binding selectivity between a quadruplex and linear or unstructured nucleic acid is 5:1 or greater. Suitably, selectivity between a quadruplex and linear or unstructured nucleic acid is 10:1 or greater, more suitably 20:1 or greater, even more suitably 50:1 or greater nM or less, and most suitably 100:1 or greater.

In one embodiment, the binding selectivity between a quadruplex and linear or unstructured nucleic acid is 5:1 or greater. Suitably, selectivity between a quadruplex and linear or unstructured nucleic acid is 10:1 or greater, more suitably 20:1 or greater, even more suitably 50:1 or greater nM or less, and most suitably 100:1 or greater.

### Salts and Solvates

The degrader of the present invention may be provided in free base form.

The degrader of the present invention may be provided in the form of a salt, preferably a pharmaceutically acceptable salt.

In some embodiments, degraders disclosed herein may be provided as salts in a protonated form together with a suitable counter anion.

Suitable counterions include both organic and inorganic anions. Example of inorganic anions include those derived from inorganic acids, including chloride (Cl⁻), bromide (Br⁻), iodide (I⁻), sulfate (SO₄), sulfite (SO₃), nitrate (NO₃), nitrite (NO₂), phosphate (PO₄), and phosphite (PO₃). Examples of organic anions include 2-acetoxybenzoate, acetate, ascorbate, aspartate, benzoate, camphorsulfonate, cinnamate, citrate, edetate, ethanedisulfonate, ethanesulfonate, formate, fumarate, gluconate, glutamate, glycolate, hydroxymalate, carboxylate, lactate, laurate, lactate, maleate, malate, methanesulfonate, oleate, oxalate, palmitate, phenylacetate, phenylsulfonate, propionate, pyruvate, salicylate, stearate, succinate, sulfanilate, tartarate, toluenesulfonate, and valerate. Examples of suitable polymeric organic anions include those derived from tannic acid and carboxymethyl cellulose.

In some embodiments, degraders disclosed herein may be provided as salts in a deprotonated form together with a suitable counter cation.

Suitable counterions include both inorganic and organic cations. Examples of suitable inorganic cations include alkali metal ions such as Na₄⁺ and K⁺, alkaline earth cations such as Ca²⁺ and Mg²⁺, and other cations such as NH₄⁺ or Al³⁺. Examples of suitable organic cations include substituted ammonium ions (e.g., NH₃R⁺, NH₂R₂⁺, NHR₃⁺, NR₄⁺). Examples of substituted ammonium ions include those derived from ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

The degraders of the present invention may be provided in the form of a solvate (a complex of solute (e.g., compound, salt of compound) and solvent). Examples of solvates include hydrates, for example, a mono-hydrate, a di-hydrate and a tri-hydrate.

The degraders of the present invention may be provided in desolvated form, for example, in dehydrated form.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a degrader of formula (I) has an asymmetric center, for example, it is bonded to four different groups, a pair of enantiomers is possible. An enantiomer can be characterized by the absolute configuration of its asymmetric center and is described by the R- and S-sequencing rules of Cahn-Ingold-Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the invention may have geometric isomeric centres (E- and Z-isomers). It is to be understood that the present invention encompasses all optical, diastereoisomers and geometric isomers and mixtures thereof that possess antiproliferative activity.

The present invention also encompasses compounds of the invention as defined herein which comprise one or more isotopic substitutions. For example, H may be in any isotopic form, including ¹H, ²H(D), and ³H (T); C may be in any isotopic form, including ¹²C, ¹³C, and ¹⁴C; and O may be in any isotopic form, including ¹⁶O and ¹⁸O; and the like.

It is also to be understood that certain compounds of the present invention may exist in solvated as well as unsolvated forms such as, for example, hydrated forms. It is to be understood that the invention encompasses all such solvated forms that possess antiproliferative activity.

It is also to be understood that certain compounds of the present invention may exhibit polymorphism, and that the invention encompasses all such forms that possess antiproliferative activity.

Compounds of the present invention may exist in a number of different tautomeric forms and references to compounds of the present invention include all such forms. For the avoidance of doubt, where a degrader of the present invention can exist in one of several tautomeric forms, and only one is specifically described or shown, all others are nevertheless embraced by the present invention. Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

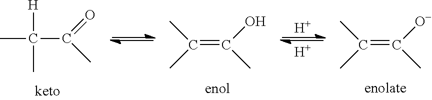

Compounds of the present invention containing an amine function may also form N-oxides. A reference herein to a degrader of the present invention that contains an amine function also includes the N-oxide. Where a degrader of the present invention contains several amine functions, one or more than one nitrogen atom may be oxidised to form an N-oxide. Particular examples of N-oxides are the N-oxides of a tertiary amine or a nitrogen atom of a nitrogen-containing heterocycle. N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (mCPBA), for example, in an inert solvent such as dichloromethane.

The compounds of the present invention may be administered in the form of a pro-drug which is broken down in the human or animal body to release a degrader of the present invention. A pro-drug may be used to alter the physical properties and/or the pharmacokinetic properties of a degrader of the invention. A pro-drug can be formed when the compound of the invention contains a suitable group or substituent to which a property-modifying group can be attached. Examples of pro-drugs include in vivo cleavable ester derivatives that may be formed at a carboxy group or a hydroxy group in a degrader of the present invention and in-vivo cleavable amide derivatives that may be formed at a carboxy group or an amino group in a compound of the present invention.

Accordingly, the present invention includes those compounds of the invention as defined hereinbefore when made available by organic synthesis and when made available within the human or animal body by way of cleavage of a pro-drug thereof. Accordingly, the present invention includes those compounds of the invention that are produced by organic synthetic means and also such compounds that are produced in the human or animal body by way of metabolism of a precursor compound, that is a degrader of the present invention may be a synthetically-produced compound or a metabolically-produced compound.

A suitable pharmaceutically acceptable pro-drug of a degrader of the present invention is one that is based on reasonable medical judgement as being suitable for administration to the human or animal body without undesirable pharmacological activities and without undue toxicity.

Various forms of pro-drug have been described, for example in the following documents:
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

A suitable pharmaceutically acceptable pro-drug of a degrader of the present invention that possesses a carboxy group is, for example, an in vivo cleavable ester thereof. An in vivo cleavable ester of a degrader of the present invention containing a carboxy group is, for example, a pharmaceutically acceptable ester which is cleaved in the human or animal body to produce the parent acid. Suitable pharmaceutically acceptable esters for carboxy include C₁₋₆ alkyl esters such as methyl, ethyl and tert-butyl, C₁₋₆alkoxymethyl esters such as methoxymethyl esters, C₁₋₆alkanoyloxymethyl esters such as pivaloyloxymethyl esters, 3-phthalidyl esters, C₃₋₆cycloalkylcarbonyloxy-C₁₋₆alkyl esters such as cyclopentylcarbonyloxymethyl and 1-cyclohexylcarbonyloxyethyl esters, 2-oxo-1,3-dioxolenylmethyl esters such as 5-methyl-2-oxo-1,3-dioxolen-4-ylmethyl esters and C₁₋₆ alkoxycarbonyloxy-C₁₋₆alkyl esters such as methoxycarbonyloxymethyl and 1-methoxycarbonyloxyethyl esters.

A suitable pharmaceutically acceptable pro-drug of a degrader of the present invention that possesses a hydroxy group is, for example, an in vivo cleavable ester or ether thereof. An in vivo cleavable ester or ether of a degrader of the present invention containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁₋₁₀alkanoyl groups such as acetyl, benzoyl, phenylacetyl and substituted benzoyl and phenylacetyl groups, C₁₋₁₀alkoxycarbonyl groups such as ethoxycarbonyl, N,N —(C₁₋₆)₂carbamoyl, 2-dialkylaminoacetyl and 2-carboxyacetyl groups.

Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₋₄ alkyl)piperazin-1-ylmethyl. Suitable pharmaceutically acceptable ether forming groups for a hydroxy group include a-acyloxyalkyl groups such as acetoxymethyl and pivaloyloxymethyl groups.

A suitable pharmaceutically acceptable pro-drug of a degrader of the present invention that possesses a carboxy group is, for example, an in vivo cleavable amide thereof, for example an amide formed with an amine such as ammonia, a C₁₋₄alkylamine such as methylamine, a (C₁₋₄alkyl)₂amine such as dimethylamine, N-ethyl-N-methylamine or diethylamine, a C₁₋₄alkoxy-C₂₋₄alkylamine such as 2-methoxyethylamine, a phenyl-C₁₋₄ alkylamine such as benzylamine and amino acids such as glycine or an ester thereof.

A suitable pharmaceutically acceptable pro-drug of a degrader of the present invention that possesses an amino group is, for example, an in vivo cleavable amide derivative thereof. Suitable pharmaceutically acceptable amides from an amino group include, for example an amide formed with C₁₋₁₀alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₋₄alkyl)piperazin-1-ylmethyl.

The in vivo effects of a degrader of the present invention may be exerted in part by one or more metabolites that are formed within the human or animal body after administration of a degrader of the formula I. As stated hereinbefore, the in vivo effects of a degrader of the present invention may also be exerted by way of metabolism of a precursor compound (a pro-drug).

Though the present invention may relate to any compound or particular group of compounds defined herein by way of optional, preferred or suitable features or otherwise in terms of particular embodiments, the present invention may also relate to any compound or particular group of compounds that specifically excludes said optional, preferred or suitable features or particular embodiments.

### Synthesis

The compounds of the present invention can be prepared by any suitable technique known in the art. Particular processes for the preparation of these compounds are described further in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

It will be appreciated that during the synthesis of the compounds of the invention in the processes defined herein, or during the synthesis of certain starting materials, it may be desirable to protect certain substituent groups to prevent their undesired reaction. The skilled chemist will appreciate when such protection is required, and how such protecting groups may be put in place, and later removed.

For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule.

Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide.

Alternatively an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia.

Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

Resins may also be used as a protecting group.

The methodology employed to synthesise a degrader of the present invention will vary depending on the nature of any substituent groups associated therewith. Suitable processes for their preparation are described further in the accompanying Examples.

Once a degrader of the present invention has been synthesised by any one of the processes defined herein, the processes may then further comprise the additional steps of:
(i) optionally removing any protecting groups present;
(ii) optionally converting the compound of the present invention into another compound of the present invention;
(iii) optionally forming a pharmaceutically acceptable salt, hydrate or solvate thereof; and/or
(iv) optionally forming a prodrug thereof.

An example of (ii) above is when a degrader of the present invention is synthesised and then one or more of the groups may be further reacted to change the nature of the group and provide an alternative compound of the present invention.

The resultant compounds of the present invention can be isolated and purified using techniques well known in the art.

### Methods for Cleaving a Target Nucleic Acid

The invention provides a method for cleaving a target nucleic acid molecule. The method comprises:
contacting the target nucleic acid molecule with a degrader of formula (I):

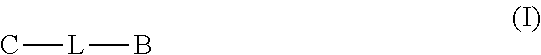

as defined herein (where —C is a cleavage group, -L- is a linker and —B is a non-covalent binding group), such that the degrader non-covalently binds to the target nucleic acid molecule; and
allowing the degrader to cleave the target nucleic acid molecule bound thereto.

Particular embodiments of the degrader of formula (I) are set out above.

In some embodiments, the target nucleic acid molecule may be contacted with the degrader in solution.

More suitably, the target nucleic acid molecule may be contacted with the degrader within a cell (i.e. intracellularly). The cell may be in vitro and may be an isolated cell, for example an isolated cell line or cell isolated from an individual (from a tissue sample, such as a biopsy).

Suitable cells may include mammalian, preferably human cells. Cells may include somatic and germ-line cells and may be at any stage of development, including fully or partially differentiated cells or non-differentiated or pluripotent cells, including stem cells, such as adult or somatic stem cells, foetal stem cells or embryonic stem cells. For example, cells may include neural cells, including neurons and glial cells, contractile muscle cells, smooth muscle cells, liver cells, hormone synthesising cells, sebaceous cells, pancreatic islet cells, adrenal cortex cells, fibroblasts, keratinocytes, endothelial and urothelial cells, osteocytes, and chondrocytes. In some embodiments, cells may be associated with a disease condition, for example cancer cells, such as carcinoma, sarcoma, lymphoma, blastoma or germ-line tumour cells, and cells with the genotype of a genetic disorder, such as Huntington's disease, cystic fibrosis, sickle cell disease, phenylketonuria, Down syndrome or Marfan syndrome.

The target nucleic acid molecule may be an endogenous nucleic acid that is present in the cell. The degrader may be an exogenous molecule. A method may comprise introducing the degrader into the cell and allowing it to bind to the target nucleic acid molecule.

The target nucleic acid molecule may be a DNA or RNA molecule. Suitable target RNA molecules may include mRNA and long non-coding RNA (lncRNA). The RNA molecule may comprise intronic and intergenic regions.

The target nucleic acid molecule may comprise a secondary or tertiary structure. Suitable secondary and tertiary structures include quadruplexes, pseudoknots, tetraloops, step-loop and hairpin loops. Preferably, the target nucleic acid molecule comprises a quadruplex or pseudoknot.

For example, a method for cleaving a target nucleic acid comprising a secondary or tertiary structure may comprise:
contacting the target nucleic acid molecule with a degrader of formula (I):

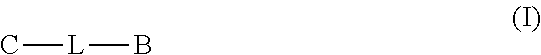

as defined herein, where —C is a cleavage group, -L- is a linker and —B is a non-covalent binding group that interacts with the secondary or tertiary structure to non-covalently bind the degrader to the target nucleic acid molecule; and
allowing the degrader to cleave the target nucleic acid molecule bound thereto.

In a particular embodiment, the secondary or tertiary structure is a quadruplex. In such cases, the method may comprise:
contacting a target nucleic acid molecule comprising a quadruplex with a degrader of formula (I):

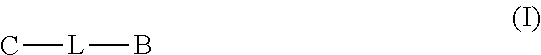

as defined herein, where —C is a cleavage group, -L- is a linker and —B is a non-covalent binding group that interacts with the quadruplex to non-covalently bind the degrader to the target nucleic acid molecule; and
allowing the degrader to cleave the target nucleic acid molecule bound thereto.

In a particular embodiment, the secondary or tertiary structure is a pseudoknot. In such cases, the method may comprise:
contacting a target nucleic acid molecule comprising a pseudoknot with a degrader of formula (I):

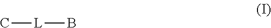

as defined herein (where —C is a cleavage group, -L- is a linker and —B is a non-covalent binding group that interacts with the pseudoknot to non-covalently bind the degrader to the target nucleic acid molecule; and
allowing the degrader to cleave the target nucleic acid molecule bound thereto.

When non-covalently bound to the target nucleic acid molecule, the degrader cleaves the target nucleic acid. Without wishing to be bound by any particular theory, the degrader is understood to cleave one or more phosphodiester bonds in the target nucleic acid.

When non-covalently bound to the target nucleic acid molecule, the cleavage group may abstract a proton from the 2′OH of a nucleotide in the target nucleic acid. Cleavage of the phosphodiester backbone may occur by intramolecular attack on the phosphate group at the 3′ position.

When non-covalently bound to the target nucleic acid molecule, the cleavage group may bind to one or more transition metals (e.g. copper). The degrader may cleave the target nucleic acid through copper-mediated nucleic acid degradation.

Binding of the degrader to the target nucleic acid may proceed via an intermediate species. That is, a target nucleic acid molecule may be cleaved as described herein by a method that comprises binding the target nucleic acid molecule to a degrader to produce an intermediate having the formula:

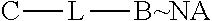

where —C is a cleavage group as defined herein, -L- is a linker as defined herein, —B is a non-covalent binding group as defined herein, ~ is a non-covalent interaction and NA is the target nucleic acid; and allowing the degrader to cleave the target nucleic acid molecule.

Particular embodiments of the degrader of formula (I) are set out above.

### Methods for Identifying Secondary or Tertiary Structures

Following selective cleavage of a target nucleic acid molecule by a degrader as described above, a method may comprise identifying the target nucleic acid molecule. This may be useful for example in the mapping of sites comprising secondary or tertiary structures within the nucleic acid.

The method may also comprise determining the abundance or amount of one or more nucleic acid molecules in a population of nucleic acids. A reduction in the abundance or amount of a nucleic acid molecule in the population relative to control is indicative that the nucleic acid molecule is the target nucleic acid molecule that has been selectively cleaved by the degrader. A suitable control may be a population of nucleic acids that has not been treated with the degrader.

Accordingly, the invention provides a method for identifying a secondary or tertiary structure within a target nucleic acid molecule, the method comprising:
providing first and second populations of nucleic acid molecules, each population comprising the target nucleic acid molecule;
introducing into the first population of nucleic acid molecules a degrader of formula (I): C-L-B (I) where —C is a cleavage group as defined herein, -L- is a linker as defined herein and —B is a non-covalent binding group as defined herein, such that the degrader non-covalently binds to the target nucleic acid molecule;
allowing the degrader to cleave the target nucleic acid molecule present in the first population; and identifying nucleic acid molecules which are present in a reduced amount in the first population relative to the second population.

Particular embodiments of the degrader of formula (I) are set out above.

The non-covalent binding group may bind to a secondary or tertiary structure within the target nucleic acid molecule. Suitable secondary or tertiary structures include quadruplexes, pseudoknots, tetraloops, step-loops and hairpin loops. Preferably, the secondary or tertiary structure is a quadruplex or pseudoknot.

The first and second populations of nucleic acid molecules may independently be isolated (ex vivo) populations of nucleic acid molecules. Alternatively, one or more of the populations of nucleic acid molecules may be present within a cell.

The method may comprise extracting the total nucleic acid, such as total DNA or total RNA, from a cell. The nucleic acid may be further analysed, for example to determine the abundance or amount of one or more nucleic acid molecules. For example, the extracted total nucleic acid may be sequenced, and the sequence reads analysed.

Suitable methods of determining the abundance or amount of nucleic acid molecules in a cell are well known in the art and include RT-qPCR, RNA-sequencing (RNA-seq), next generation (NGS), nanopore sequencing, and other sequencing techniques, such as Sanger sequencing, Tracking Indels by Composition (TIDE) (Brinkman et al Nucleic Acids Res. 2014 Dec. 16; 42(22): e168) and PCR analysis. In some embodiments, a method may comprise extracting nucleic acid molecules from the cell, sequencing the extracted nucleic acid molecules and determining the number of sequence reads (i.e. read count) for each extracted nucleic molecule to determine the abundance or amount of each nucleic acid molecule in the cell. In some embodiments, the raw read count may be normalised and expressed in RPKM (reads per kilobase of exon model per million reads) or FPKM (fragments per kilobase of exon model per million reads mapped). Suitable methods of sequencing and sequence analysis are well established in the art.

### Use in Medicine

The selective cleavage of a target nucleic acid molecules by the degrader described above may alter downstream effects of the target nucleic acid molecule. This may be useful, for example, in the treatment or prophylaxis of a disease mediated by the target nucleic acid molecule.

Accordingly, the present invention provides a degrader of formula (I) for use in a method of treatment of the human or animal body by therapy, for example, for use in a method of treatment of a disorder (e.g., a disease).

Another aspect of the present invention pertains to a method of treatment, for example, a method of treatment of a disorder (e.g., a disease), comprising administering a therapeutically-effective amount of a degrader of formula (I) to a subject in need of treatment.

Another aspect of the present invention pertains to use of degrader of formula (I) in the manufacture of a medicament for use in treating a disorder (e.g., a disease). Typically, the medicament comprises the degrader of formula (I).

### Disorders Treated

(i) Proliferative Disorders (e.g. Cancer)

According to a further aspect of the present invention, there is provided a method of inhibiting cell proliferation, in vitro or in vivo, said method comprising contacting a cell with an effective amount of a degrader of formula (I) or a pharmaceutically acceptable salt, hydrate or solvate thereof as defined herein, or a pharmaceutical composition as defined herein.

According to a further aspect of the present invention, there is provided a method of treating a proliferative disorder in a patient in need of such treatment, said method comprising administering to said patient a therapeutically effective amount of a degrader of formula (I) or a pharmaceutically acceptable salt, hydrate or solvate thereof as defined herein, or a pharmaceutical composition as defined herein.

According to a further aspect of the present invention, there is provided a method of treating cancer in a patient in need of such treatment, said method comprising administering to said patient a therapeutically effective amount of a degrader of formula (I) or a pharmaceutically acceptable salt, hydrate or solvate thereof as defined herein, or a pharmaceutical composition as defined herein.

According to a further aspect of the present invention, there is provided a degrader of formula (I) or a pharmaceutically acceptable salt, hydrate or solvate thereof as defined herein, or a pharmaceutical composition as defined herein, for use in the treatment of a proliferative condition.

According to a further aspect of the present invention, there is provided a degrader of formula (I), or a pharmaceutically acceptable salt, hydrate or solvate thereof, or a pharmaceutical composition as defined herein for use in the treatment of cancer. In a particular embodiment, the cancer is human cancer.

According to a further aspect of the present invention, there is provided the use of a degrader of formula (I), or a pharmaceutically acceptable salt, hydrate or solvate thereof, as defined herein in the manufacture of a medicament for the treatment of a proliferative condition.

According to a further aspect of the present invention, there is provide the use of a degrader of formula (I), or a pharmaceutically acceptable salt, hydrate or solvate thereof, as defined herein in the manufacture of a medicament for the treatment of cancer. Suitably, the cancer is a human cancer.

Optionally, the compound or pharmaceutical composition is administered in combination with one or more additional antiproliferative agents (e.g. checkpoint inhibitors and/or cytotoxic agents).

The term “proliferative disorder” is used herein and pertain to an unwanted or uncontrolled cellular proliferation of excessive or abnormal cells which is undesired, such as, neoplastic or hyperplastic growth, whether in vitro or in vivo. Examples of proliferative conditions include, but are not limited to, pre-malignant and malignant cellular proliferation, including, but not limited to, malignant neoplasms and tumours, cancers, leukemias, psoriasis, bone diseases, fibroproliferative disorders (e.g., of connective tissues), and atherosclerosis. Any type of cell may be treated, including but not limited to, lung, colon, breast, ovarian, prostate, liver, pancreas, brain, and skin.

When the proliferative disorder is a cancer, the cancer is optionally selected from adenoid cystic carcinoma, adrenal gland cancer, amyloidosis, anal cancer, ataxia-telangiectasia, atypical mole syndrome, basal cell carcinoma, bile duct cancer, Birt Hogg Dube Syndrome, bladder cancer, bone cancer, brain tumor, breast cancer (male and female), carcinoid tumor, cervical cancer, colorectal cancer, ductal carcinoma, endometrial cancer, esophageal cancer, gastric cancer, gastrontestinal stromal tumor (GIST), islet cell tumor, juvenile polyposis syndrome, kidney cancer, laryngeal cancer, acute lymphoblastic Leukemia leukemia, acute lymphocytic leukemia (ALL), acute myeloid leukemia (AML), adult leukemia, childhood leukemia, chronic lymphocytic leukemia (CLL), chronic myeloid leukemia (CML), liver cancer, lobular carcinoma, non-small cell lung cancer, small cell lung cancer, Hodgkin's lymphoma, non-Hodgkin's lymphoma, malignant glioma, melanoma, meningioma, multiple myeloma, myelodysplastic syndrome (MDS), nasopharyngeal cancer, neuroendocrine tumor, oral cancer, osteosarcoma, ovarian cancer, pancreatic cancer, pancreatic neuroendocrine tumors, parathyroid cancer, penile cancer, peritoneal cancer, Peutz-Jeghers Syndrome, pituitary gland tumor, polycythemia vera, prostate cancer, renal cell carcinoma, retinoblastoma, salivary gland cancer, sarcoma, kaposi sarcoma, skin cancer, small intestine cancer, stomach cancer, testicular cancer, thymoma, thyroid cancer, uterine (endometrial) cancer, vaginal cancer, or Wilms' Tumor.

In an embodiment, the degraders of formula I may target SNVs and indels that get transcribed in various cancers. For example, the rs4430796 SNP on HNF1B is associated with ovarian and prostate cancers; the rs28897672 SNV on BRCA1 is associated with ovarian cancer; rs80359351 deletion on BRCA2 is associated with breast and ovarian cancers. All of these genetic changes are reflected in respective mRNAs thus are potentially targetable with the degraders of the present invention.

Particular cancers of interest include Ewings Sarcoma (by targeting ESWR1, e.g. with a pyridostatin (PDS) non-covalent binding group) and lung, breast, cervical and colon cancers (by targeting MALAT1 with MALAT1 binding non-covalent binding groups.

### (ii) Bacterial and Viral Infections

In one embodiment (e.g., of use in methods of therapy, of use in the manufacture of medicaments, of methods of treatment), the treatment is treatment of a bacterial or viral infection.

Suitably, the viral infection is an infection with an RNA virus (e.g., a virus in which the viral genome comprises single- or double-stranded RNA). Many pathogenic viruses exploit a −1 ribosomal frameshifting as a mechanism for correct translation of proteins and this phenomenon is enabled by secondary RNA structures such as stem-loops and pseudoknots.

Accordingly, targeting these secondary RNA structures with a degrader of formula (I) can cleave and inactivate the viral RNA, and treat the viral infection.

Examples of RNA viruses include (+)ssRNA viruses such as coronaviruses, picornaviruses and togaviruses; (−)ssRNA viruses such as orthomyxoviruses and rhabdoviruses; and dsRNA viruses such as reoviruses.

Preferably, the virus is a (+)ssRNA virus, more preferably a coronavirus. Examples of coronaviruses include alphacoronaviruses such as transmissible gastroenteritis virus, feline coronavirus, canine coronavirus; betacoronaviruses such as middle east respiratory syndrome-related coronavirus (MERS-CoV), murine coronavirus (M-CoV) and severe acute respiratory syndrome-related coronavirus (SARS-CoV, SARS-CoV-2); gammacoronavirus such as avian coronavirus; and deltacoronavirus such as bulbul coronavirus HKU11 and porcine coronavirus HKU15.

The bacterial infection may be an infection with a Gram-negative or Gram-positive bacterium. Both classes of bacteria contain a bacterial ribosome, which is a riboenzyme comprising both protein and RNA units. Accordingly, targeting the RNA units with a degrader of formula (I) can cleave and inactive the bacterial ribosome, and treat the bacterial infection.

Examples of medically-relevant Gram-negative bacteria include Haemophilus influenzae, Kiebsiella pneumoniae, Legionella pneumophila and Pseudomonas aeruginosa (which are primarily associated with respiratory problems); Escherichia coli and Enterobacter cloacae (which are primarily associated with urinary problems); and Helicobacter pylori and Salmonella enterica (which are primarily associated with gastrointestinal problems), Neisseria meningitidis (which is primarily associated with meningitis).

Accordingly, in one embodiment, the Gram-negative bacterial species is selected from the group consisting of E. coli, E. cloacae, H. pylori, S. enterica, H. influenzae, K. pneumoniae, L. pneumoniae, L. pneumophila, P. aeruginosa and N. meningitidis.

Examples of medically-relevant Gram-positive bacteria include actinomyces, bacillus, clostridium, corynebacterium (e.g. Corynebacterium diphtheriae), enterococcus, erysipelothrix, listerial (e.g. Listeria monocytogenes), nocardia, staphylococcal, and streptococcal (e.g. Staphylococcus aureus).

Accordingly, in one embodiment, the Gram-negative bacterial genus is selected from the group consisting of actinomyces, bacillus, clostridium, corynebacterium, enterococcus, erysipelothrix, listerial nocardia, staphylococcal, and streptococcal.

In one embodiment (e.g., of use in methods of therapy, of use in the manufacture of medicaments, of methods of treatment), the treatment is treatment of a respiratory tract infection, a urinary tract infection, or gastroenteritis.

### Other Conditions

The degraders of the present invention may also be used to degrade other nucleic acid sequences associated with other disease states. For example, the degrader sof the present invention may be used to treat nucleotide repeat disorders (e.g. Huntington's, Fragile X, Myotonic Dystrophy Type 1), and sequences of mRNAs encoding non-structured proteins (e.g. IAPP in Type II Diabetes).

### Patients Treated

In one embodiment (e.g., of use in methods of therapy, of use in the manufacture of medicaments, of methods of treatment), the treatment is administered to a subject in need of treatment.

The subject in need of treatment (the patient) may be a chordate, a vertebrate, a mammal, a placental mammal, a marsupial (e.g., kangaroo, wombat), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutang, gibbon), or a human.

The subject in need of treatment may be an adult or juvenile.

Preferably, the subject in need of treatment is a human, more preferably an adult human.

Alternatively, the subject in need of treatment is a non-human animal used in laboratory research. Preferably, the non-human animal is a rodent (e.g., a guinea pig, a hamster, a rat, a mouse).

### Routes of Administration

In one embodiment (e.g., of use in methods of therapy, of use in the manufacture of medicaments, of methods of treatment), the treatment is administered by any convenient route of administration, whether systemically/peripherally or topically (i.e., at the site of desired action).

The routes of administration may be oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eyedrops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### Formulations

In one embodiment (e.g., of use in methods of therapy, of use in the manufacture of medicaments, of methods of treatment), the degrader of formula (I) is administered alone. Typically, however, it is preferable to present the degrader in a pharmaceutical formulation (e.g., composition, preparation, medicament) comprising at least one degrader as described herein, together with one or more other pharmaceutically acceptable ingredients well known to those skilled in the art, including, but not limited to, pharmaceutically acceptable carriers, diluents, excipients, adjuvants, fillers, buffers, preservatives, anti-oxidants, lubricants, stabilisers, solubilisers, surfactants (e.g., wetting agents), masking agents, colouring agents, flavouring agents, and sweetening agents. The formulation may further comprise other active agents, for example, other therapeutic or prophylactic agents.

Thus, the present invention further provides pharmaceutical compositions, and methods of making a pharmaceutical composition comprising mixing at least one degrader described herein, together with one or more other pharmaceutically acceptable ingredients well known to those skilled in the art, e.g., carriers, diluents, excipients, etc. If formulated as discrete units (e.g., tablets, etc.), each unit contains a predetermined amount (dosage) of the compound.

The term “pharmaceutically acceptable” as used herein, pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

Suitable carriers, diluents, excipients, etc. can be found in standard pharmaceutical texts, for example, Remington's Pharmaceutical Sciences, 18th edition, Mack Publishing Company, Easton, Pa., 1990; and Handbook of Pharmaceutical Excipients, 5th edition, 25 2005.

The formulations may be prepared by any methods well known in the art of pharmacy. Such methods include the step of bringing into association the degrader with a carrier which constitutes one or more accessory ingredients. In general, the formulations are prepared by uniformly mixing the degrader with a carrier (e.g., a liquid carrier, a finely divided solid carrier, etc.), and then shaping the product, if necessary.

The formulation may be prepared to provide for rapid or slow release; immediate, delayed, timed, or sustained release; or a combination thereof.

Formulations may suitably be in the form of liquids, solutions (e.g., aqueous, nonaqueous), suspensions (e.g., aqueous, non-aqueous), emulsions (e.g., oil-in-water, water-in-oil), elixirs, syrups, electuaries, mouthwashes, drops, tablets (including, e.g., coated tablets), granules, powders, losenges, pastilles, capsules (including, e.g., hard and soft gelatin capsules), cachets, pills, ampoules, boluses, suppositories, pessaries, tinctures, gels, pastes, ointments, creams, lotions, oils, foams, sprays, mists, or aerosols.

Formulations may suitably be provided as a patch, adhesive plaster, bandage, dressing, or the like which is impregnated with one or more compounds and optionally one or more other pharmaceutically acceptable ingredients, including, for example, penetration, permeation, and absorption enhancers. Formulations may also suitably be provided in the form of a depot or reservoir.

The degrader may be dissolved in, suspended in, or mixed with one or more other pharmaceutically acceptable ingredients. The compound may be presented in a liposome or other microparticulate which is designed to target the compound, for example, to blood components or one or more organs.

### Dosages

In one embodiment (e.g., of use in methods of therapy, of use in the manufacture of medicaments, of methods of treatment), the treatment comprises administering a therapeutically-effective amount of a degrader of formula (I) to a subject in need of treatment.

It will be appreciated by one of skill in the art that appropriate dosages of the degraders described herein, and compositions comprising the degraders, can vary from patient to patient. Determining the optimal dosage will generally involve the balancing of the level of therapeutic benefit against any risk or deleterious side effects. The selected dosage level will depend on a variety of factors including, but not limited to, the activity of the particular degrader, the route of administration, the time of administration, the rate of excretion of the degrader, the duration of the treatment, other drugs, compounds, and/or materials used in combination, the severity of the disorder, and the species, sex, age, weight, condition, general health, and prior medical history of the patient. The amount of degrader and route of administration will ultimately be at the discretion of the physician, veterinarian, or clinician, although generally the dosage will be selected to achieve local concentrations at the site of action which achieve the desired effect without causing substantial harmful or deleterious side-effects.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

In general, a suitable dose of the degrader is in the range of about 10 μg to about 250 mg (more typically about 100 μg to about 25 mg) per kilogram body weight of the subject per day.

Where the compound is a salt, an ester, an amide, a prodrug, or the like, the amount administered is calculated on the basis of the parent compound and so the actual weight to be used is increased proportionately.

### Other Preferences

Each and every compatible combination of the embodiments described above is explicitly disclosed herein, as if each and every combination was individually and explicitly recited.

Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein

Unless context dictates otherwise, the descriptions and definitions of the features set out above are not limited to any particular aspect or embodiment of the invention and apply equally to all aspects and embodiments which are described.

### EXAMPLES

Certain aspects and embodiments of the invention will now be illustrated by way of example and with reference to the figures described above.

### Illustrative Example 1

### Studies to Show the Concept of Non-Covalent Nucleic Acid Degradation Using a Model Degrader Comprising an Imidazole Cleavage Group

### General Experimental Protocols

### In Vitro Pseudoknot Oligo Degradation Reactions

RNA oligo (20 μM) was added to a pH 7.5 HEPES (20 mM) buffer supplemented with KCl (50 mM) and EDTA (10 mM). The mixture was incubated at 37° C. for 30 min. MTDB-deg 16a, MTDB or TDB-deg 16b (1 mM) was then added. The reaction mixture was incubated at 37° C. for 3 h and then kept at 4° C. The reaction mixtures were analyzed by LC-MS or gel electrophoresis.

### LC-MS Analysis of Oligonucleotides.

Oligonucleotides were analyzed by LC-MS following the method of Mikutis et al., 2020.

Oligomers were analysed using a Xevo G2-S TOF mass spectrometer coupled to an Acquity UPLC system using an Acquity UPLC BEH C18 1.7 μm column. The system utilises electronspray (ESI) ionisation. Two mobile phases were used—16.3 mM TEA, 400 mM HFIP in H2O and 16.3 mM TEA, 400 mM HFIP in 80:20 v/v MeCN and H2O, with a flow rate of 0.200 mL/min. Calibration curves for the RNA species were based either on A260 or intensities of specified negative m/z signals. Intensities of integrated peaks were calculated using native modules of KNIME software platform (33). Total mass spectra were reconstructed from the ion series using the MaxEnt algorithm preinstalled on MassLynx software (v. 4.1 from Waters) according to the manufacturer's instructions. To obtain the negative ion series described, the oligomer peak in the chromatogram was selected for integration and further analysis.

RNA degradation gel electrophoresis

Gel electrophoresis was carried out following the method of Mikutis et al., 2020.

In vitro RNA degradation reactions were carried out as described above. The quenched reaction mixture was mixed in 1:1 ratio with a loading buffer (95% formamide, 0.025% SDS, 0.025% bromophenol blue (BPB), 0.025% xylene cyanol FF, 0.025% ethidium bromide, 0.5 mM EDTA), heated at 70° C. for 5 min, and cooled to 0° C. PAGE was performed on Novex™ TBEUrea Gels, containing 15% polyacrylamide under 1 x TBE buffer (89 mM Tris, 89 mM boric acid, 2 mM EDTA) at 180 V for 60 min. Gel staining was performed using SYBR Green II RNA Gel Stain (Invitrogen) in 1x TBE buffer. The stained RNA was visualized with ChemiDoc MP (Bio-Rad, United Kingdom).

### Viral Stocks

SARS-CoV-2 stocks used to infect Vero CCL-81 cells were established from passage 4 of SARS-CoV-2 isolated from a Portuguese patient (internal reference: 606_IMM ID-5452) at approximately 1.7×10⁶ PFU/mL, after 4 days in Vero CCL-81 culture. Stock titers were calculated by plaque assay. Briefly, approximately 8×10⁵ CCL-81 cells/well were seeded in 6-well plates and allowed to grow to confluence for 24 h. Medium was removed, and 500 μL of 10-fold serial dilutions of virus-containing supernatants were adsorbed in duplicate for 1 h, at 37° C. Plates were rocked manually to redistribute inoculum every 15 minutes. Cells were overlaid with 1.25% carboxymethylcellulose (CMC) in supplemented DMEM and incubated at 37° C. for 4 days. After incubation, the CMC overlay was removed, and cells were fixed with 4% formaldehyde/PBS and stained with 0.1% toluidine blue. After inactivation by fixation, plates were sealed with parafilm and disinfected before being removed from the BSC and BSL3. Viral plaques were counted to determine infectious titers (PFU (plaque forming units)/mL).

### Viral Infection of Vero CCL-81

Vero CCL-81 cells at 80% confluency were incubated with SARS-CoV-2 inoculum for 1 h at 37° C. After incubation, the inoculum was removed and DMEM medium supplemented with 2.5% FCS was added for 24 h, or until samples were harvested.

### Gel Electrophoresis Analysis

500 ng of SARS-CoV-2 RNA was incubated with or without 100 μM of MTDB-deg 16a in 1x HEPES buffer for 2 h at 37° C. with mild agitation. The samples were then analyzed on a 1.5% agarose gel.

### Nanopore Sequencing

500 ng of SARS-CoV-2 RNA was incubated with or without 100 μM of MTDB-deg 16a in 1x HEPES buffer for 2 h at 37° C. with mild agitation. The samples were then prepared for sequencing following the manufacturer's protocol for Direct RNA Sequencing (SQK-RNA002, ONT). The prepared libraries were loaded on FLO-MIN106D flow cells (ONT) and sequenced on a MinION Mk1C device (ONT).

Genomic sequence of the Wuhan-hu1 strain of SARS-CoV-2 (GenBank: MN908947.3) and the genomic annotation (NC_045512.2) were downloaded from the NCBI database. Sequence reads were aligned to the Wuhan-hu1 genome using minimap2 (Li et al., 2018). with parameters “-ax splice —N32-un-k13”. CIGAR strings of the alignments were processed by customized scripts. Reads were flagged as leader if the splice junction within the read starts between the first 60-120 bp of the genome. Reads were assigned to individual transcript if it covers more than 90% of the annotated transcript or more than 90% or the read sequence lies within the transcript.

### Drug Assay to Determine 50% Inhibitory Concentration

Increasing concentrations of MTDB-deg 16a (ranging from 0.07 to 25 μM) were tested to determine the 50% inhibitory concentration (IC₅₀). Vehicle (H₂O) control and control molecules were included in parallel. Cells were seeded in 96 well-plates at approximately 40% confluency 24 h before infection. MTDB-deg 16a, MTDB or TDB-deg 16b were added either 1 h before infection or 1 h after infection. SARS-CoV-2 cryopreserved stocks, were thawed at room temperature and used to infect cells at a 0.05 multiplicity of infection (MOI). Inhibition of viral growth was measured by harvesting cells at 24 h after infection. Viral growth was assessed by measuring viral loads by PCR targeting the E gene and pseudoknot region.

### Detection of Viral Plaque-Forming Units by Plaque Assay

Approximately 8×10⁵ CCL-81 cells/well were seeded in 6-well plates and allowed to grow to 80% confluence for 24 h. Supernatant of cultures treated with the compounds was diluted in DMEM medium supplemented with 2.5% FCS and added to pre-seeded wells of a 6-well plate and incubated for 1 h, at 37° C. Plates were rocked manually to redistribute inoculum every 15 min. Cells were overlaid with 1.25% CMC in supplemented DMEM and incubated at 37° C. for 4 days. After incubation, the CMC overlay was removed, and cells were fixed with 4% formaldehyde/PBS and stained with 0.1% toluidine blue. After inactivation by fixation, plates were sealed with parafilm and disinfected before being removed from the BSC and BSL3. Viral plaques were counted to determine infectious titers (PFU (plaque forming units)/mL).

### Quantification of Viral Load by PCR

Cell pellets were harvested into 300 μL of lysis buffer. Viral RNA was extracted by using a NZY Viral RNA Isolation kit (NZYtech) and cDNA was synthesized by using NZY First-Strand cDNA Synthesis kit (NZYtech) following manufactures' instructions. The quantitative RT-PCR (RT-qPCR) was then performed by using PowerUp SYBR Green Master Mix (BIO-RAD), set up by Applied Biosystems RT-PCR 7500Fast machine with default SYBR green program.

The primers used for detecting SARS-CoV-2 were:

E gene:
5′-ACAGGTACGTTAATAGTTAATAGCGT-3′
(forward),
5′-ATATTGCAGCAGTACGCACACA-3′
(reverse);
N gene:
5′-GACCCCAAAATCAGCGAAAT-3′
(forward),
5′-TCTGGTTACTGCCAGTTGAATCTG-3′
(reverse);
Pseudo-knot:
5′-CCGCGAACCCATGCTTCAGTCA-3′
(forward),
5′-CACGGTGTAAGACGGGCTGCAC-3′
(reverse);
18S:
5′-GTAACCCGTTGAACCCCATT-3′
(forward),
5′-CCATCCAATCGGTAGTAGCG-3′
(reverse).

### Viral Recovery Assay

Two sets of samples were prepared for the recovery assay, where cells at 80% confluency were infected with SARS-CoV-2 cryopreserved stocks at a 0.05 MOI for 2 h. Then, the inoculum was removed, and infected cells were incubated with the MTDB-deg 16a, MTDB, and TDB-deg 16b at 6 μM for 24 h, at 37° C. and 5% CO₂. After 24 h, in one set of samples cells were harvested into lysis buffer for PCR analysis of viral growth. On the other set of samples, compounds in supernatant were removed, replaced by drug-free media and incubated for an additional 24 h, at 37° C. and 5% CO₂. After 24 h of incubation (corresponding to 48 h timepoint) cells were harvested into lysis buffer and viral growth was be measured by PCR targeting the E gene and pseudoknot region. Percentage of viral recovery was normalised to vehicle control.

### Cytotoxicity Assay

To determine if the compounds were toxic to cells, 1×10⁴ Vero E6 cells per well were seeded in 96-well plates. After 24 h, cells were incubated with increasing concentrations of MTDB-deg 16a, MTDB or TDB-deg 16b (ranging from 0.05 μM to 2 μM). The viability of cells after 24 h of incubation with the compounds was assessed by using CellTiter Blue viability assay (Promega) in accordance with manufacturer's protocol. Briefly, Cell titer blue stock solution was diluted 1:20. A volume of 80 L of diluted cell titer blue was added to each well and incubated at 37° C., for 2 h.

### Dynamic Light Scattering (DLS)

Stock solutions (10 mM) of each screening molecule were prepared in neat DMSO and sequentially diluted in water to a final concentration of 25 or 12.5 μM. Data were collected on a Zetasizer Nano S (Malvern) at 25° C.

### Antiviral Activity on an Animal Model of SARS-CoV-2 Infection

Ten to twelve week-old specific pathogen-free Hemizygous for Tg(K18-ACE2)2Prlmn (Strain B6.Cg-Tg(K18-ACE2)2Prlmn/J, the Jackson laboratory strain 034860) mice were used in this study. Mice were intranasally infected with 1×10⁴ PFU of SARS-CoV-2 in 50 μl of PBS. Compounds were administrated intranasally 1 hour pre-infection and 3 hours post-infection. Mice were treated either with vehicle (n=6), MTDB-degrader 16a at 25 mg/kg (n=6), MTDB at 10 mg/kg (n=3) or TDB-degrader 16b at 25 mg/kg (n=5). On day 5 post SARS-CoV-2 infection, animals were humanely euthanized and left lung was harvested for viral quantification by plaque assay and right lung was harvested for histopathological analysis.

### Western Blot Analysis.

For in vitro experiments, samples were treated with vehicle (H₂O) or MTDB-deg 16a (6 mM) for 24 hours. Cells were then lysed using whole cell lysis buffer (50 mM Tris-HCl pH=8.0, 450 mM NaCl, 0.1% NP-40, 1 mM EDTA), supplemented with 1 mM DTT, protease inhibitors (Sigma), and phosphatase inhibitors (Sigma). For in vivo experiments, the whole left lung from mice was homogenized in 3 mL of DMEM and 750 μL was transferred to an equal volume of whole cell lysis buffer, supplemented as above. Protein concentrations were accessed using Bradford Assays (BioRad). Prior to loading the samples were supplemented with LDS Loading Buffer (Life technologies) and Sample Reducing Agent (Life Technologies). 40 μg of protein was separated on SDS-PAGE gels and blotted onto polyvinylidene difluoride (PVDF) membranes (GE Healthcare). Western Blot experiments were performed using the following antibodies: anti-beta actin (Abcam, ab8224), anti phospho-MAPKAPK-2 (Thr334) (27B7) (Cell Signalling, 3007), anti-Phospho-p38 MAPK (Thr180/Tyr182) (D3F9) XP® (Cell Signalling, 4511), goat anti-mouse IgG H&L (HRP) (Abcam, ab205719) and goat-anti rabbit HRP (Abcam, ab6721).

### Synthesis of Azido-Imidazoles

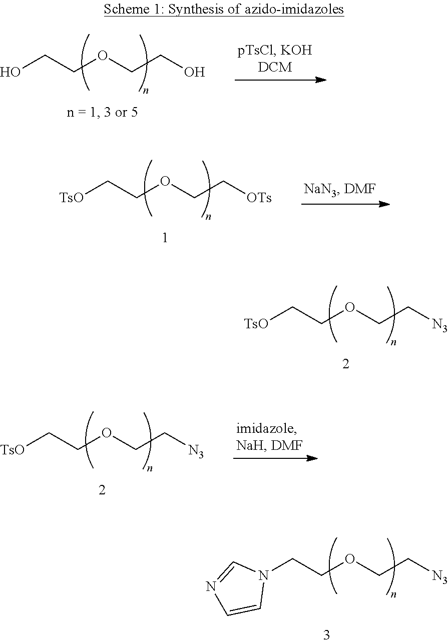

### Synthesis of Hexaethylene glycol di(p-toluenesulfonate) (1)

Hexaethylene glycol (1.0 mmol) was solved in DCM (10 mL) and p-toluenesulfonyl chloride (2.2 mmol) and KOH (10 mmol) were added at 0° C. The reaction mixture was stirred for 6 hours at room temperature, then filtered and washed with water. After drying over MgSO₄, the solvent was evaporated under reduced pressure. No further purification necessary. Yield: 95% (colourless oil).

¹H NMR (400 MHz, CDCl₃): δ_{H} 7.78 (d, 4H), 7.33 (d, 4H), 4.14 (t, 4H), 3.67 (br tr, 4H, 3.60 (br s, 8H), 3.57 (br s, 8H), 2.43 (br s, 6H). MS: m/z for C₂₆H₃₉O₁₁S₂: 591.19.

The physical and spectroscopic data were in agreement with that described in the literature (Mikutis et al., 2020).

### Synthesis of Hexaethylene glycol p-toluenesulfonate azide (2a)

Hexaethylene glycol di(p-toluenesulfonate) 1 (1.0 mmol) was dissolved in DMF (10 mL) and sodium azide (1.0 mmol) was added. The reaction mixture was stirred at 60° C. for 6 hours, then cooled down to room temperature and stirred over-night. The mixture was washed with brine and dried over MgSO₄. To remove DMF, toluene was added and the solvent was evaporated under reduced pressure. The crude product was purified via column chromatography (EtOAc:Hexane, 1:1). Yield: 54% (colourless oil).

¹H NMR (400 MHz, CDC₃) δ_{H} 7.82 (d, 2H), 7.36 (d, 2H), 4.18 (t, 2H), 3.59-3.73 (20H, PEG), 3.41 (t, 2H), 2.47 (s, 3H). MS: m/z for C₁₉H₃₁N₃NaO₈S: 484.2.

The physical and spectroscopic data were in agreement with that described in the literature (Mikutis et al., 2020).

### Synthesis of Tetraethylene glycol p-toluenesulfonate azide (2b)

Tetraethylene glycol di(p-toluenesulfonate) (2.7 g, 5.4 mmol) was dissolved in anhydrous DMF (10 ml). Sodium azide (355 mg, 5.4 mmol) was added and the mixture was placed under N₂ and stirred for 18 h at 55° C. The solvent was removed in vacuo, and the products were purified via flash column chromatography (3:1 Pet. Ether:AcOEt to 1:1 Pet. Ether:AcOEt). The product was obtained as a colourless oil (798 mg, 2.1 mmol, 39%).

¹H NMR (400 MHz, CDCl3) δ 7.82 (d, 2H), 7.37 (d, 2H), 4.19 (t, 1H), 3.60-3.73 (12H, PEG), 3.40 (t, 2H), 2.47 (s, 3H). MS: m/z for C₁₅H₂₃N₃NaO₆S 396.1207.

### Synthesis of Diethylene glycol p-toluenesulfonate azide (2c)

Diethylene glycol di(p-toluenesulfonate) (1.0 mmol) was dissolved in DMF (10 mL) and sodium azide (1.0 mmol) was added. The reaction mixture was stirred at 60° C. for 6 hours, then cooled down to room temperature and stirred over-night. The mixture was washed with brine and dried over MgSO₄. To remove DMF, toluene was added and the solvent was evaporated under reduced pressure. The crude product was purified via column chromatography (EtOAc:Hexane, 1:1). Yield: 59% (colourless oil).

¹H NMR (400 MHz, CDCl3) 5H 7.80 (d, 2H), 7.35 (d, 2H), 4.17 (t, 2), 3.70 (t, 2H), 3.61 (t, 2H), 3.32 (t, 2H), 2.45 (s, 3H). MS: m/z for C₁₁H₁₅N₃NaO₄S: 308.068.

The physical and spectroscopic data were in agreement with that described in the literature (Mikutis et al., 2020).

### Synthesis of Hexaethylene glycol imidazolate azide (3a)

Imidazole (1.0 mmol) was dissolved under inert conditions in dry DMF (15 mL) and sodium hydride (60% dispersion in mineral oil, 1.2 mmol) was added. After stirring for 30 min at 0° C. 2a (1.0 mmol) was added. The reaction mixture was stirred at 60° C. overnight and, after cooling to room temperature, the mixture was quenched with water (20 mL). Subsequent extraction with EtOAc and DCM, drying over MgSO₄ and evaporation of the solvent under reduced pressure gave the crude product. The crude product was then purified via column chromatography (EtOAc:MeCOH, 3:1). Yield: 35% (colourless oil).

¹H NMR (400 MHz, CDCl₃) δ_{H} 7.52 (s, 1H), 7.02 (s, 1H), 6.98 (s, 1H), 4.09 (t, 2H), 3.72 (t, 2H), 3.55-3.78 (18H, PEG), 3.36 (t, 2H). MS: m/z for C₁₅H₂₈N₅O₅ 358.21.

The physical and spectroscopic data were in agreement with that described in the literature (Mikutis et al., 2020).

### Synthesis of Tetraethylene glycol imidazolate azide (3b)

Imidazole (18 mg, 0.27 mmol) and NaH (60% dispersion in mineral oil, 12 mg, 0.27 mmol) were suspended in anhydrous DMF (1 ml) at 0° C.. The mixture was placed under N₂ atmosphere, allowed to warm to room temperature and stirred for 30 min. 2b (100 mg, 0.27 mmol) was dissolved in anhydrous DMF (1 ml) and the resulting solution was added to the first mixture. It was then stirred for 20 h at 55° C. Solvent was then removed in vacuo and the resulting residue was purified via flash chromatography (dry loading, gradient EtOAC to 9:1 EtOAc:MeOH). The product was obtained as a colourless oil (54 mg, 0.20 mmol, 74%).

¹H NMR (400 MHz, CDCl₃) δ 7.55 (s, 1H), 7.05 (s, 1H), 7.05 (s, 1H), 4.12 (t, 2H), 3.75 (t, 2H), 3.60-3.71 (10H, PEG), 3.39 (t, 2H). ¹³C NMR (100 MHz, CDCl₃) δ_{C} 137.6, 129.2, 119.4, 70.5-70.7 (multiple PEG peaks), 70.0, 50.7, 47.1. MS: m/z for C₁₁H₁₉N₅O₃ 270.1582.

### Synthesis of Diethylene glycol imidazolate azide (3c)

Imidazole (1.0 mmol) was dissolved under inert conditions in dry DMF (15 mL) and sodium hydride (60% dispersion in mineral oil, 1.2 mmol) was added. After stirring for 30 min at 0° C., 2c (1.0 mmol) was added. The reaction mixture was stirred at 60° C. overnight and then cooled to room temperature. The mixture was quenched with water (20 mL). After extraction with EtOAc and DCM, drying over MgSO₄, and evaporation of the solvent under reduced pressure, a crude product was obtained. The crude product was then purified via column chromatography (EtOAc:MeCOH, 3:1). Yield: 38% (colourless oil).

¹H NMR (400 MHz, CDCl₃) δ_{H} 7.53 (s, 1H), 7.06 (s, 1H), 6.99 (s, 1H), 4.14 (t, 2H), 3.75 (t, 2H), 3.60 (t, 2H), 3.36 (t, 2H). MS: m/z for C₁₁H₁₉N₅O₃ 182.1

The physical and spectroscopic data were in agreement with that described in the literature (Mikutis et al., 2020).

### Synthesis azido-ethyl imidazole (4)

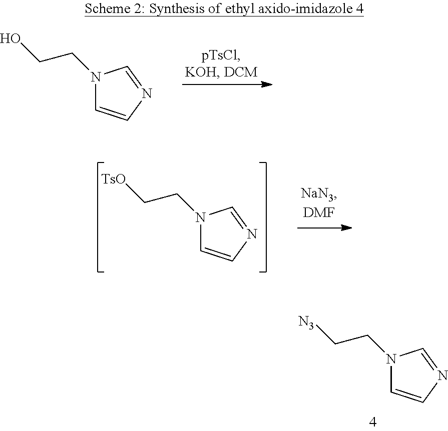

Hydroxyethyl imidazole (1.0 mmol) was dissolved in DCM (10 mL) at 0° C. and KOH (10 mmol) and p-toluenesulfonyl chloride (1.2 mmol) were added. The reaction mixture was stirred for 6 hours at room temperature, then filtered and the solvent was removed under reduced pressure. The crude was resolved in DMF and sodium azide (1.0 mmol) was added. It was stirred at 60° C. for 6 hours, then cooled down to room temperature and stirred over-night. Toluene was added and the solvent was removed under reduced pressure. Purification was performed via column chromatography (EtOAC:MeOH, 3:1). Yield: 26% (white solid).

¹H NMR (400 MHz, CDC₃) δ 7.51 (s, 1H), 7.09 (s, 1H), 6.96 (s, 1H), 4.09 (t, J=5.7 Hz, 1H), 3.62 (t, J=5.7 Hz, 1H).

### Synthesis of Pyridostatin Degraders

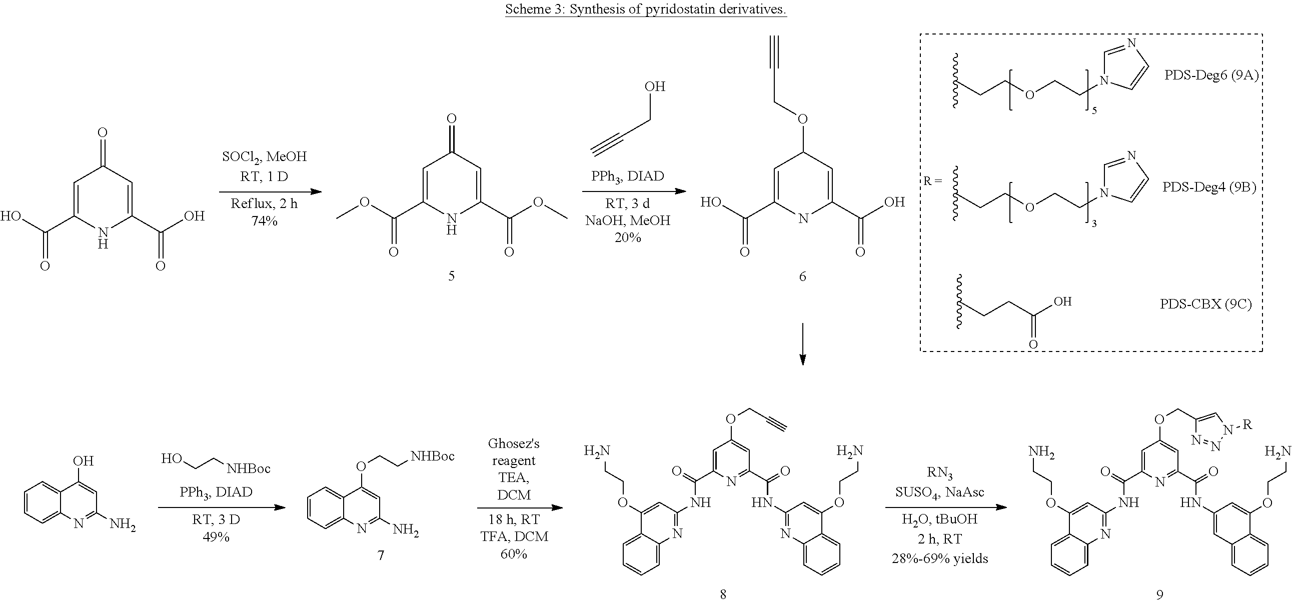

### Synthesis of Chelidamic acid dimethyl ester (5)

Chelidamic acid hydrate (2.0 g, 11 mmol) was suspended in 20 ml MeOH. Thionyl chloride (500 μL, 6.9 mmol) was added dropwise to the suspension at −10° C. A white to brown colour change was observed. The solution was warmed to RT and stirred overnight. The brown solution was refluxed for 2 h and the solvent removed in vacuo. The brown crude product was then re-crystallised from EtOH, resulting in a beige solid chelidamic acid dimethyl ester 5 (864 mg, 3.9 mmol, 36%).

¹H NMR (400 MHz, DMSO) δ 11.77 (br s, 1H), 7.61 (s, 2H), 3.88 (s, 6H). ¹³C NMR (101 MHz, DMSO) δ 165.97, 164.88, 149.37, 115.33, 52.68. HRMS (ES) calculated for C₉H₁₀NO₅ ([M+H]⁺) m/z: 212.0559, found 212.0567.

### Synthesis of Propargylic chelidamic acid (6)

Chelidamic acid dimethyl ester 5 (0.82 g, 3.8 mmol), propargyl alcohol (0.33 mL, 5.7 mmol) and polymer bound triphenylphosphine (3.47 g, 1.5 mmol loading/g, 5.2 mmol) were suspended in 55 mL freshly distilled THF. The solution was degassed using freeze-pump-thaw cycling and cooled to 0° C., DIAD (1.0 mL, 5.1 mmol) was added dropwise under argon. The solution was warmed to RT and stirred for 3d. The solution was filtered, and the solvent removed in vacuo. Chelidamic acid dimethyl ester was obtained via column chromatography (50% EtOAc, 50% pet. ether). It was then was dissolved in 50 mL MeOH, followed by addition of 50 mL aqueous NaOH (0.33 g, 7.5 mmol) solution. The resulting mixture was stirred for 5 min and deprotection was confirmed by TLC. The organic solvent was removed in vacuo. 5% formic acid was added to acidify followed by extraction with EtOAC 3×100 mL. The organic layer was then dried with MgSO₄, filtered and the solvent removed in vacuo. This yielded an off-white solid propargylic chelidamic acid 6 (0.17 g, 0.77 mmol, 20%).

¹H NMR (400 MHz, MeOD) δ 7.93 (s, 2H), 5.02 (d, J=2.4 Hz, 2H), 3.15 (t, J=2.5 Hz, 1H). ¹³C NMR (100 MHz, MeOD) δ 168.12, 166.98, 150.54, 150.43, 115.73, 78.97, 77.90, 77.88, 57.74, 57.68. HRMS (ES) calculated for C₁₀H₃NO₅([M+H]⁺) m/z: 222.0397, found 222.0391.

### Synthesis of O-(Ethyl-2-N-boc-amine)-2-aminoquinolinone (7)

2-aminoquinolinone (1.0 g, 6.2 mmol), N-boc ethanolamine (1.4 mL, 9.1 mmol) and triphenylphosphine (3.3 g, 13 mmol) were dissolved in 10 mL freshly distilled THF. The solution was degassed using freeze-pump-thaw cycling and cooled to 0° C., DIAD (1.8 mL, 9.2 mmol) was added dropwise under argon. The solution warmed to RT and stirred for 3d. The solvent was then removed in vacuo. The product was purified by a gradient column chromatography with 100% EtOAc to 90% EtOAc, 10% MeOH. The solvent was removed in vacuo to obtain an off-white solid 7 (552 mg, 1.82 mmol, 29%).

¹H NMR (400 MHz, CDCl₃) δ 7.98 (dd, J=8.0, 1.0 Hz, 2H), 7.60 (dd, J=8.4, 1.2 Hz, 2H), 7.55 (ddd, J=8.5, 6.7, 1.6 Hz, 2H), 7.23 (ddd, J=8.1, 6.6, 1.3 Hz, 2H), 6.04 (s, 1H), 5.01 (br s, 1H), 4.69 (br s, 2H), 4.18 (t, J=5.1 Hz, 4H), 3.68 (q, J=5.5 Hz, 4H), 1.46 (s, 9H). ¹³C NMR (100 MHz, CDCl₃) δ 162.32, 158.13, 155.90, 148.55, 130.25, 125.63, 121.97, 121.60, 117.52, 90.09, 79.83, 67.52, 39.82, 28.38. HRMS (ES) calculated for C₁₆H₂₂N₃O₃ ([M+H]⁺) m/z: 304.1661, found 304.1649.

### Synthesis of Alkyne-Pyridostatin (8)

Propargylic chelidamic acid 6 (0.12 g, 0.54 mol) was dissolved in 1.2 mL DCM. Then Ghosez reagent (170 μL, 1.3 mmol) was added dropwise at 0° C. The orange solution was then stirred at RT for 2 h. The chlorination was confirmed by TLC. Triethylamine (0.18 mL, 1.3 mmol) was added dropwise at 0° C. The solution was then stirred at RT for 1h. 7 (0.37 g, 1.2 mmol) was suspended in 1.2 mL DCM and then added dropwise to the mixture. The mixture turned brown-red and was stirred under argon overnight. The crude protected product 8a (not shown) was precipitated from hot MeCN as a red solid. The red solid 8a was then dissolved in DCM. A 2:1 mixture of DCM:TFA was added to acidify the solution and remove the N-boc protection. The solvent was removed in vacuo and the product was purified via HPLC (gradient 100% H₂O, 0.1% FA to 100% MeCN, 0.1% FA). Lyophilization afforded an off-white solid Alkyne-Pyridostatin 8 (51 mg, 86 μmol, 16%).

HRMS (ES) calculated for C₃₂H₃ON₇O₅ ([M+H]⁺) m/z: 592.2308, found 592.2327.

### Synthesis of Pyridostatin Degraders (9A to 9C)

Alkyne-Pyridostatin 8 (15 mg, 25 μmol) was dissolved in 2.5 mL of a 2:1 mixture of H₂O:tBuOH. A solution of copper sulfate pentahydrate (250 μL, 100 mM, 25 μmol) was added followed by a solution of sodium ascorbate (1.3 mL, 100 mM, 130 μmol). The cloudy yellow solution was degassed and stirred for 10 mins. A solution of the appropriate azido-imidazole (3a, 3b or 3-azidopropionic acid) (3.8 mL, 10 mM) was then added. The solution was stirred under argon for 2 h. The organic solvent was removed in vacuo. Then the product was purified via HPLC (gradient 100% H₂O, 0.1% FA to 100% MeCN, 0.1% FA). The product was obtained as a white or an off-white solid.

9A (PDS-deg6). 48% yield (11.3 mg, 12 μmol). HRMS (ES) calculated for C47H52N12O10 ([M+H]+) m/z: 949.4321, found 949.4344.

9B (PDS-deg4). 69% yield (14.8 mg, 17 μmol). HRMS (ES) calculated for C43H49N12O8 ([M+H]+) m/z: 861.3796, found 861.376.

9C (PDS-CBX). 28% yield (4.9 mg, 6.9 μmol). HRMS (ES) calculated for C35H34N10O7 ([M+H]⁺) m/z: 707.2690, found: 707.2684.

### Synthesis of Pseudoknot-Dearader

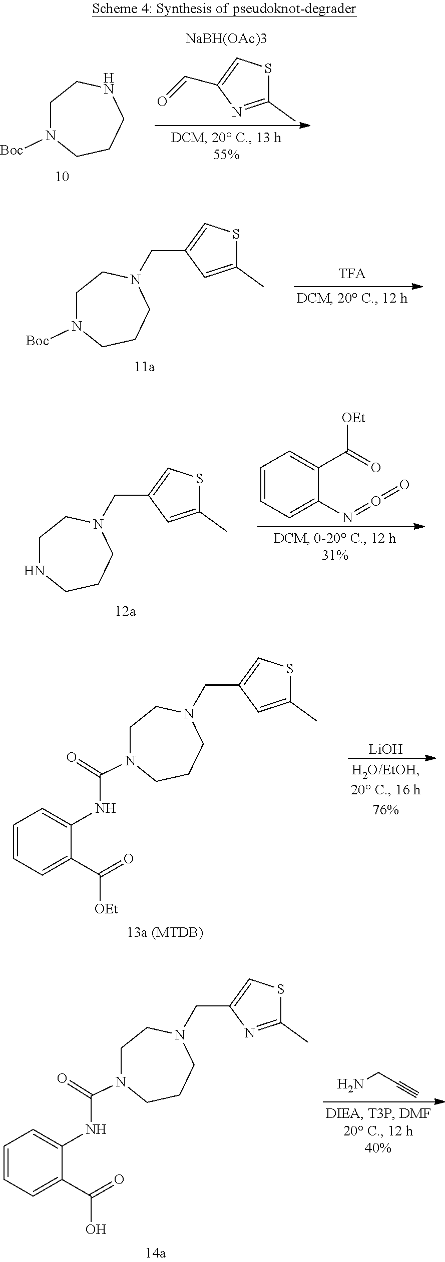

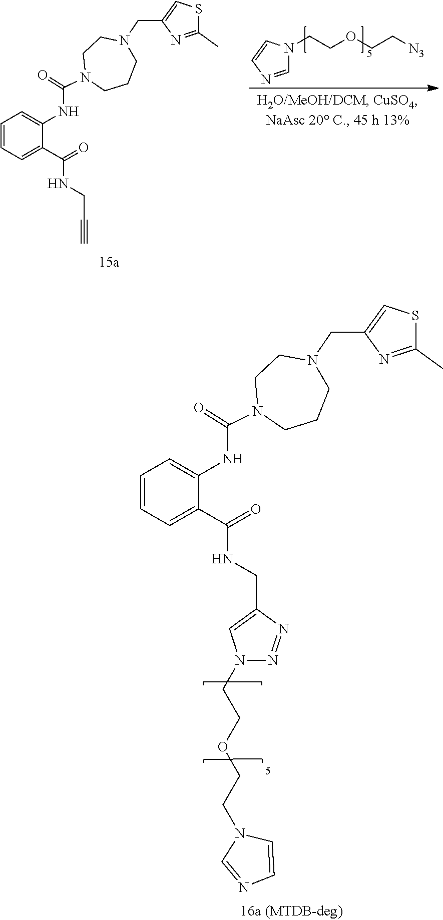

### Synthesis of compound 11a

2-methylthiazole-4-carbaldehyde (10.0 g, 78.6 mmol, 1.0 equiv.) in DCM (100 mL) was added to compound 10 (16.5 g, 82.6 mmol, 16.2 mL, 1.1 equiv.) in one portion at 25° C. under N₂. The mixture was stirred at 25° C. for 3 h. To the mixture was added NaBH(OAc)₃ (25.0 g, 118 mmol, 1.5 equiv.) and stirred for 10 h. The residue was poured into water (50 mL) and stirred for 10 min. The aqueous phase was extracted with DCM (3×20 mL). The combined organic phase was dried with anhydrous Na₂SO₄ and filtered; the solvent was removed in vacuo. The product was purified via column chromatography (gradient, petroleum ether to petroleum ether/ethyl acetate 10/1) to yield compound 11a (13.5 g, 43.4 mmol, 55% yield) as a yellow oil.

LCMS [+scan]: calculated m/z C₂₀H₂₆N₃O₃S 388.2; observed 388.1.

### Synthesis of compound 12a

TFA (40.0 g, 351 mmol, 26 mL, 8.4 equiv.) was added to compound 11a (13.0 g, 41.7 mmol, 1.0 equiv.) in DCM (130 mL) at 25° C. at N₂. The mixture was stirred for 12 h. The solvent was removed in vacuo to give a TFA salt of compound 12a (23.0 g, crude) as a red oil.

LCMS [+scan]: calculated m/z C₁₀H₂₈N₃S 212.1; observed 212.0.

### Synthesis of MTDB (compound 13a)

To a solution of compound 12a (20.0 g, 45.5 mmol, 1.0 equiv.) in DCM (200 mL) was added TEA (9.21 g, 91.0 mmol, 12.7 mL, 2.0 equiv.) at 20° C. under N₂. Then ethyl 2-isocyanatobenzoate (8.70 g, 45.5 mmol, 1.0 equiv.) was added to the mixture at 0° C. The mixture was stirred at 20° C. for 12 h. The solvent was removed in vacuo. The residue was purified by column chromatography (gradient, petroleum ether/ethyl acetate 100/1 to 20/1) to give MTDB (compound 13a 5.62 g, 14.0 mmol, 31% yield) as an off-white solid.

¹H NMR (400 MHz, CD₃OD): δ 8.42 (br d, J=8.4 Hz, 1H), 8.08 (br d, J=8.0 Hz, 1H), 7.62 (s, 1H), 7.52-7.59 (m, 1H), 7.09 (br t, J=7.6 Hz, 1H), 4.34-4.46 (m, 4H), 3.93 (br s, 2H), 3.77 (br t, J=6.0 Hz, 2H), 3.49 (br s, 4H), 3.28-3.31 (m, 1H), 2.74 (s, 3H), 2.31 (br d, J=4.8 Hz, 2H), 1.43 (t, J=7.2 Hz, 3H). LCMS [+scan]: calculated m/z C₂₀H₂₇N₄O₃S 403.2; observed 403.1.

### Synthesis of compound 14a

To a mixture of MTDB (compound 13a 5.60 g, 13.9 mmol, 1.0 equiv.) in EtOH (120 mL) and H₂O (30 mL) was added LiOH monohydrate (2.34 g, 55.7 mmol, 4.0 equiv.) at 25° C. under N₂. The mixture was stirred at 25° C. for 12 h. The mixture was adjusted to pH 6 with 1 M HCl and the aqueous phase was extracted with ethyl acetate (3×40 mL); then the organic phase was dried with anhydrous Na₂SO₄, filtered and concentrate in vacuo to give compound 14a (2.60 g, 6.94 mmol, 50%) as a yellow solid.

LCMS [+scan]: calculated m/z C₁₈H₂₃N₄O₃S 375.1; observed 375.1.

### Synthesis of compound 15a

To a mixture of compound 14a (2.60 g, 6.94 mmol, 1.0 equiv.) and propargyl amine (1.15 g, 20.8 mmol, 1.33 mL, 3.0 equiv.) in DMF (200 mL) was added DIEA (4.49 g, 34.7 mmol, 6.05 mL, 5.00 equiv.) at 25° C. under N₂. The mixture was added T₃P (4.42 g, 13.9 mmol, 4.13 mL, 2.0 equiv.) and stirred at 50° C. for 12 h. The mixture was poured into water (200 mL) and the aqueous phase was extracted with ethyl acetate (3×70 mL); then the organic phase was washed with brine (60 mL); the organic phase was dried with anhydrous Na₂SO₄, filtered and the solvent was removed in vacuo. The residue was purified by column chromatography (gradient, petroleum ether/ethyl acetate 100/1 to ethyl acetate) to yield 15a (1.20 g, 2.80 mmol, 40% yield).

¹H NMR (400 MHz, CDCl₃): δ 10.54 (br s, 1H), 8.46 (d, J=8.2 Hz, 1H), 7.43-7.51 (m, 2H), 7.11-7.11 (m, 1H), 6.99 (q, J=7.6 Hz, 2H), 6.49 (br s, 1H), 4.22 (dd, J=5.2, 2.6 Hz, 2H), 3.72-3.83 (m, 4H), 3.64-3.69 (m, 2H), 2.77-2.96 (m, 4H), 2.72 (s, 3H), 2.32 (t, J=2.6 Hz, 1H), 2.04 (br d, J=15.2 Hz, 2H). LCMS [+scan]: calculated m/z C₂₁H₂₆N₅O₂S 412.2; observed 412.0.

### Synthesis of MTDB-deg (compound 16a)

A mixture of azido-imidazole 3a (200 mg, 280 μmol, 1.0 equiv.), compound 15a (115 mg, 280 μmol, 1.0 equiv.) and CuSO₄ (22.3 mg, 140 μmol, 21.5 uL, 0.5 equiv.) in DCM (5 mL), MeOH (5 mL) and H₂O (5 mL) were stirred at 20° C. for 0.5 h; then NaAsc (11.1 mg, 55.9 μmol, 0.2 equiv.) was added to the mixture and stirred at 20° C. for 4.5 h. The mixture was diluted with H₂O (10 mL) and then extracted with DCM (3×10 mL). The combined organic phase was dried over anhydrous Na₂SO₄, filtered and concentrated in vacuo. The residue was purified by via HPLC (column: Phenomenex Gemini-NX 80×40 mm×3 um; mobile phase: [water (10 mM NH₄HCO₃)-ACN]; B %: 15%-35%, 8 min) to give MTDB-deg 16a (28.0 mg, 34.9 μmol, 13% yield) as a light yellow oil.

¹H NMR (400 MHz, DMSO-d₆): δ11.03 (s, 1H), 9.29 (br t, J=5.2 Hz, 1H), 8.37 (d, J=8.4 Hz, 1H), 7.96 (s, 1H), 7.74 (br d, J=7.2 Hz, 1H), 7.63 (br s, 1H), 7.43 (t, J=7.60 Hz, 1H), 7.15-7.31 (m, 2H), 6.99 (t, J=7.6 Hz, 1H), 6.89 (br s, 1H), 4.46-4.53 (m, 4H), 4.10 (br t, J=5.2 Hz, 2H), 3.77-3.81 (m, 2H), 3.71 (br s, 2H), 3.66 (br t, J=5.07 Hz, 2H), 3.44-3.57 (m, 20H), 3.34 (s, 25H), 2.73 (br s, 1H), 2.62 (br s, 6H), 1.84 (br s, 2H). HRMS [+scan]: calculated m/z C₃₆H₅₃N₁₀O₇S 769.3819; observed 769.3830.

### Synthesis of Control Degrader for Pseudoknot Experiments

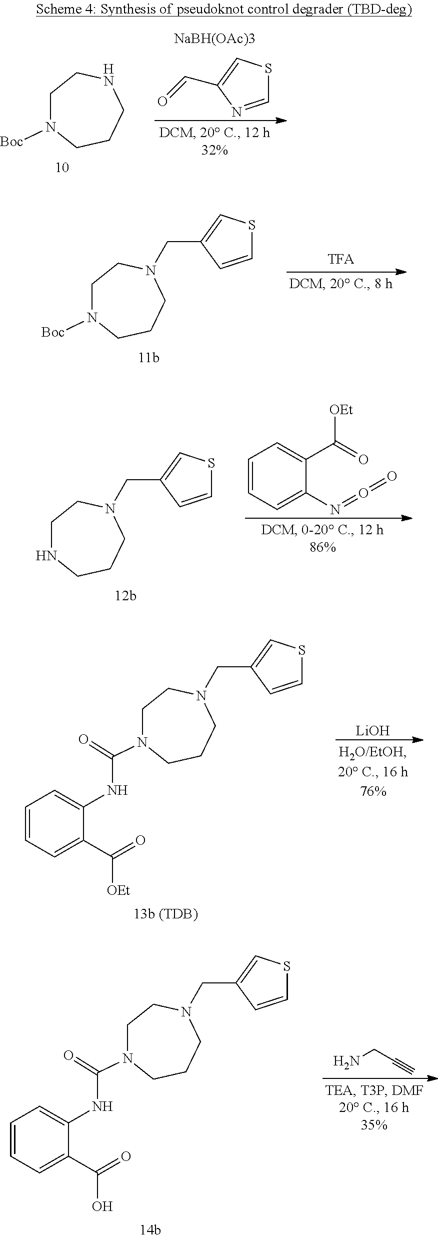

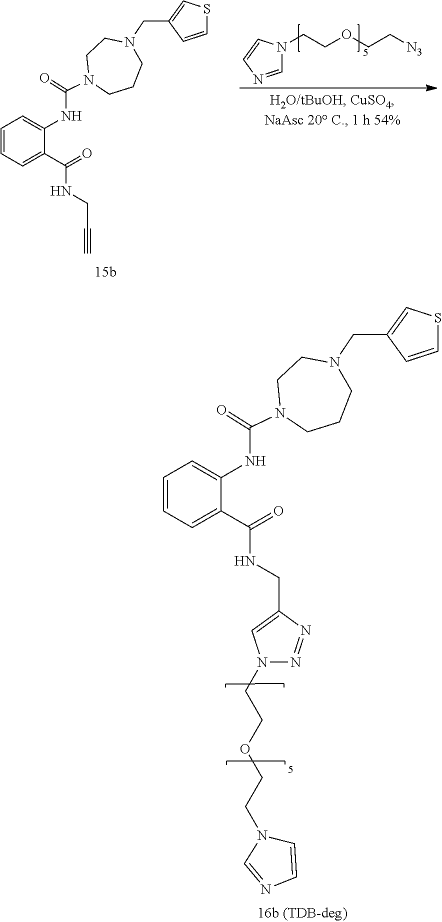

### Synthesis of compound 11b

To thiophene-3-carbaldehyde (2.00 g, 17.8 mmol, 1.63 mL, 1.0 equiv.) in DCM (80 mL) was added compound 10 (3.93 g, 19.6 mmol, 3.85 mL, 1.1 equiv.) at 20° C. under N₂. The mixture was stirred at 20° C. for 3 h. Then NaBH(OAc)₃ (5.67 g, 26.8 mmol, 1.5 equiv.) was added to the mixture at 0° C. and stirred at 20° C. for 10 h. The reaction mixture was quenched by addition of water (60 mL) and extracted with DCM (2×20 mL). The combined organic layers were washed with brine (30 mL), dried over anhydrous Na₂SO₄, filtered and concentrated in vacuo. The residue was purified by silica gel chromatography (gradient petroleum ether/ethyl acetate=100/1 to 20/1) to give compound 11b (1.70 g, 5.73 mmol, 32% yield) as a red oil.

¹H NMR (400 MHz, CDCl₃): δ 7.21-7.31 (m, 1H), 7.03-7.15 (m, 2H), 3.64 (s, 2H), 3.37-3.54 (m, 4H), 2.55-2.68 (m, 4H), 1.81 (br dd, J=10.8, 4.9 Hz, 2H), 1.40-1.52 (m, 9H).

### Synthesis of compound 12b

To a solution of compound 11b (1.70 g, 5.73 mmol, 1.0 equiv.) in DCM (20 mL) was added TFA (6.16 g, 54.0 mmol, 4.00 mL, 9.4 equiv.) at 20° C. under N₂. The mixture was stirred at 20° C. for 8 h. The solvent was removed in vacuo to yield a TFA salt of compound 12b (3.00 g, crude) as a red oil which was used in the next step without further purification.

### Synthesis of TDB (compound 13b)

To a mixture of compound 12b (3.00 g, 9.67 mmol, 1.0 equiv.) in DCM (40 mL) was added TEA (1.96 g, 19.3 mmol, 2.69 mL, 2.0 equiv.) at 20° C. under N₂. Then ethyl 2-isocyanatobenzoate (1.85 g, 9.67 mmol, 1.0 equiv.) was added to the mixture at 0° C. The mixture was stirred at 20° C. for 12 h. The solvent was removed in vacuo. The residue was purified by column chromatography (gradient, Petroleum ether/Ethyl acetate=100/1 to 20/1) to give TDB 13b (3.30 g, 8.35 mmol, 86% yield) as an off-white solid.

¹H NMR (400 MHz, CDC₃): δ10.59 (s, 1H), 8.52 (d, J=8.4 Hz, 1H), 7.94 (dd, J=8.0, 1.53 Hz, 1H), 7.42 (t, J=7.6 Hz, 1H), 7.14-7.24 (m, 1H), 6.96-7.07 (m, 2H), 6.88 (t, J=7.2 Hz, 1H), 4.28 (q, J=7.2 Hz, 2H), 3.55-3.67 (m, 6H), 2.70 (br s, 2H), 2.53-2.65 (m, 2H), 1.91 (br s, 2H), 1.33 (t, J=7.2 Hz, 3H). LCMS [+scan]: calculated m/z C₂₀H₂₆N₃O₃S 388.2; observed 388.1.

### Synthesis of compound 14b

To a mixture of TDB 21 (100 mg, 0.26 mmol, 1.0 equiv.) in EtOH (1.2 mL) and H₂O (1.2 mL) was added LiOH monohydrate (65 g, 1.5 mmol, 6.0 equiv.) at 25° C. under N₂. The mixture was stirred at 25° C. for 16 h, after which time additional LiOH monohydrate (130 g, 3.1 mmol, 12.0 equiv.) was added. After 2 h the mixture was adjusted to pH 6 with 1 M HCl and the aqueous phase was extracted with DCM (3×10 mL); then the organic phase was dried with anhydrous MgSO₄, filtered and concentrate in vacuo to give compound 14b (70 mg, 0.19 mmol, 76%) as a yellow oily solid.

¹H NMR (400 MHz, CD₃OD): δ_{H} 8.41 (d, J=8.5 Hz, 1H), 8.08 (d, J=8.5 Hz, 1H), 7.76 (br s, 1H), 7.64 (m, 1H), 7.53 (t, J=7.5 Hz, 1H), 7.32 (d, J=4.5 Hz, 1H), 7.07 (t, J=7.5 Hz, 1H), 4.46 (br s, 2H), 3.95 (m, 2H), 3.75 (tr, J=6.0 Hz, 2H), 3.47 (m, 4H), 2.34 (m, 2H). ¹³C NMR (100 MHz, CD₃OD) δ_{C} 170.9, 155.4, 142.7, 133.7, 131.2, 129.7, 129.3, 128.8, 127.6, 121.2, 119.0, 115.3, 55.2, 54.9, 53.3, 44.5, 40.2, 24.0. HRMS [+scan]: calculated m/z C₁₈H₂₂N₃O₃S 360.1382; observed 360.1386.

### Synthesis of compound 15b

To a mixture of compound 14b (70 mg, 195 μmol, 1.0 equiv.) and propargyl amine (11.2 mg, 200 μmol, 13.1 μL, 1.0 equiv.) in DMF (3 mL) was added TEA (75.5 mg, 74.6 μmol, 104 μL, 4.0 equiv.) at 25° C. under N₂. The mixture was added 50% T₃P in DMF (238 mg, 370 μmol, 1.9 equiv.) and stirred at 25° C. for 16 h. The solvent was removed in vacuo and the title compound was purified on a column (gradient DCM to DCM:MeOH 9:1). To remove residual DMF, the compound was dissolved in DCM (10 mL) and washed with H₂O (10 mL) and 1% aqueous NaOH solution (10 mL). The organic phase was dried with anhydrous MgSO₄, the solvent was removed in vacuo to afford 15b (26 mg, 66 μmol, 34% yield, ratio of 4:1).

¹H NMR (400 MHz, CDCl₃, reported for major diastereomer): δ 10.42 (br s, 1H), 8.35 (d, J=8.9 Hz, 1H), 7.36-7.41 (m, 2H), 7.26 (m, 1H), 7.11 (m, 1H), 7.06 (m, 1H), 6.87-6.97 (m, 2H), 6.49 (br s, 1H), 4.16 (dd, J=5.2, 2.5 Hz, 2H), 3.59-3.71 (m, 6H), 2.75 (br s, 2H), 2.65 (t, J=5.5 Hz, 2H), 2.27 (t, J=2.5 Hz, 1H), 1.96 (br s, 2H). ¹³C NMR (100 MHz, CDCl₃) δ_{C} 169.4, 155.4, 141.5, 140.0, 132.5, 128.4, 126.8, 125.5, 122.7, 121.1, 120.9, 118.9, 79.2, 71.8, 57.4, 55.0, 46.0, 29.6. HRMS [+scan]: calculated m/z C₃₆H₅₂N₉O₇S 397.1698; observed 397.1716.

### Synthesis of TDB-deg (compound 16b)

Compound 15b (9.9 mg, 25 μmol, 1.0 equiv.) was dissolved in a mixture of H₂O (1.7 mL) and tBuOH (0.8 mL). Aqueous CuSO₄ solution (250 μL, 100 mM, 25 μmol, 1.0 equiv.) was added, followed by aqueous NaAsc solution (1.3 mL, 100 mM, 130 μmol, 5.2 equiv.). The resulting cloudy yellow mixture was put under argon atmosphere; aqeous solution of azido-imidazole 3a (3.8 mL, 10 mM, 38 μmol, 1.5 equiv.) was then added. The reaction was stirred at room temperature for 1 h, after which they reation mixture turned clear yellow. The reaction was quenched with disodium EDTA dihydrate (9.3 mg, 25 μmol, 1 equiv.), the organic solvent was removed in vacuo and the mixture was purified via HPLC. The fractions containing the product were lyophilised, resulting in TDB-deg (15b) as a yellow-brown oily solid (10.2 mg, 14 μmol, 54% yield).

HRMS [+scan]: calculated m/z C₃₆H₂₂N₉O₇S 754.3710; observed 754.3698.

### Synthesis of Chloramphenicol Degraders

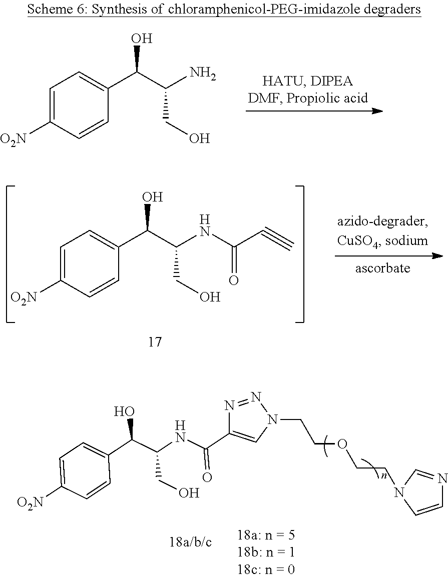

### Synthesis of chloramphenicol-6PEG-imidazole degrader (18a)

Propiolic acid (1.1 mmol) was dissolved in DMF (20 mL) under inert conditions and cooled to 0° C. HATU (2.0 mmol) and DIPEA (2.0 mmol) were added and the reaction mixture was stirred for 30 min at 0° C. (1R,2R)-(−)-2-Amino-1-(4-nitrophenyl)-1,3-propanediol (1.0 mmol) was added and the reaction mixture was stirred for 24 hours at room temperature. After filtration the crude mixture was directly used for the copper-click reaction. Azido-imidazole 3a (1.0 mmol), CuSO₄ (5 mol %), sodium ascorbate (0.2 mmol) were added, and the reaction mixture was stirred for another 24 hours at room temperature. After addition of toluene, the solvent was evaporated under reduced pressure. The crude product was purified via prep-HPLC. Yield: 10% (yellow oil).

¹H NMR (400 MHz, MeOD) δ 8.29 (s, 1H), 8.14 (d, J=8.8 Hz, 2H), 7.79 (s, 1H), 7.67 (d, J=8.7 Hz, 2H), 7.44 (s, 1H), 7.22 (s, 1H), 5.23 (d, J=2.6 Hz, 1H), 4.61-4.56 (m, 2H), 4.34 (s, 1H), 4.22 (t, J=4.9 Hz, 2H), 3.89-3.82 (m, 2H), 3.79-3.68 (m, 2H), 3.62-3.50 (m, 18H).

MS: m/z for C₂₇H₃₉N₇O₁₀: 621.3.

### Synthesis of chloramphenicol-2PEG-imidazole degrader (18b)

Propiolic acid (1.1 mmol) was dissolved in DMF (20 mL) under inert conditions and cooled to 0° C. HATU (2.0 mmol) and DIPEA (2.0 mmol) were added and the reaction mixture was stirred for 30 min at 0° C. (1R,2R)-(−)-2-Amino-1-(4-nitrophenyl)-1,3-propanediol (1.0 mmol) was added and the reaction mixture was stirred for 24 hours at room temperature. After filtration the crude mixture was directly used for the copper-click reaction. Azido-imidazole 3c (1.0 mmol), CuSO₄ (5 mol %), sodium ascorbate (0.2 mmol) were added, and the reaction mixture was stirred for another 24 hours at room temperature. After addition of toluene, the solvent was evaporated under reduced pressure. The crude product was purified via prep-HPLC. Yield: 10% (white solid).

¹H NMR (400 MHz, MeOD) δ 8.19 (s, 1H), 8.16 (d, J=8.8 Hz, 2H), 7.69 (d, J=8.6 Hz, 2H), 7.55 (s, 1H), 7.00 (s, 1H), 6.88 (s, 1H), 5.25 (d, J=2.8 Hz, 1H), 4.60 (dd, J=5.5, 4.5 Hz, 2H), 4.38 (ddd, J=7.0, 5.9, 2.9 Hz, 1H), 4.14 (dd, J=5.5, 4.4 Hz, 2H), 3.89 (dd, J=11.0, 7.0 Hz, 1H), 3.84 (t, J=5.1 Hz, 2H), 3.75 (dd, J=10.9, 5.9 Hz, 1H), 3.71 (t, J=4.9 Hz, 2H). MS: m/z for C₁₉H₂₃N₇O₆: 445.2.

### Synthesis of chloramphenicol-ethyl-imidazole degrader (18c)

Propiolic acid (1.1 mmol) was dissolved in DMF (20 mL) under inert conditions and cooled to 0° C. HATU (2.0 mmol) and DIPEA (2.0 mmol) were added and the reaction mixture was stirred for 30 min at 0° C. (1R,2R)-(−)-2-Amino-1-(4-nitrophenyl)-1,3-propanediol (1.0 mmol) was added and the reaction mixture was stirred for 24 hours at room temperature. After filtration the crude mixture was directly used for the copper-click reaction. Azido-imidazole 4 (1.0 mmol), CuSO₄ (5 mol %), sodium ascorbate (0.2 mmol) were added, and the reaction mixture was stirred for another 24 hours at room temperature. After addition of toluene, the solvent was evaporated under reduced pressure. The crude product was purified via prep-HPLC. Yield: 12% (white solid).

¹H NMR (400 MHz, MeOD) δ 8.14 (d, J=8.8 Hz, 2H), 8.03 (s, 1H), 7.64 (d, J=8.3 Hz, 2H), 7.43 (s, 1H), 6.97 (s, 1H), 6.93 (s, 1H), 5.21 (d, J=2.7 Hz, 1H), 4.82 (dd, J=6.8, 5.0 Hz, 2H), 4.58 (dd, J=6.7, 4.9 Hz, 2H), 4.31 (ddd, J=7.2, 5.9, 2.8 Hz, 1H), 3.84 (dd, J=10.9, 7.2 Hz, 1H), 3.69 (dd, J=10.9, 5.9 Hz, 1H). MS: m/z for C₁₇H₁₉N₇O₅: 401,1.

### Targeting of G-Quadruplexes and Degradation of RNA

Two degraders were rationally designed to target the RNA G-quadruplex (rG4) by joining the known G4 binder pyridostatin with azido-imidazoles of different lengths (9A, 9B).

The copper-induced azide-alkyne cycloaddition (CuAAC) used to join the two components tolerates a vast array of substrates and results in triazole, a bioisostere of an amide and a moiety well-tolerated in biological systems. We utilised the same strategy to synthesise CBX-PDS—a pyridostatin derivative that is known to selectively bind RNA, not DNA, G-quadruplexes. The two rG4 degraders and a binding control were tested in vitro and in cellular systems.

### In Vitro Degradation of rG4 Oligomers

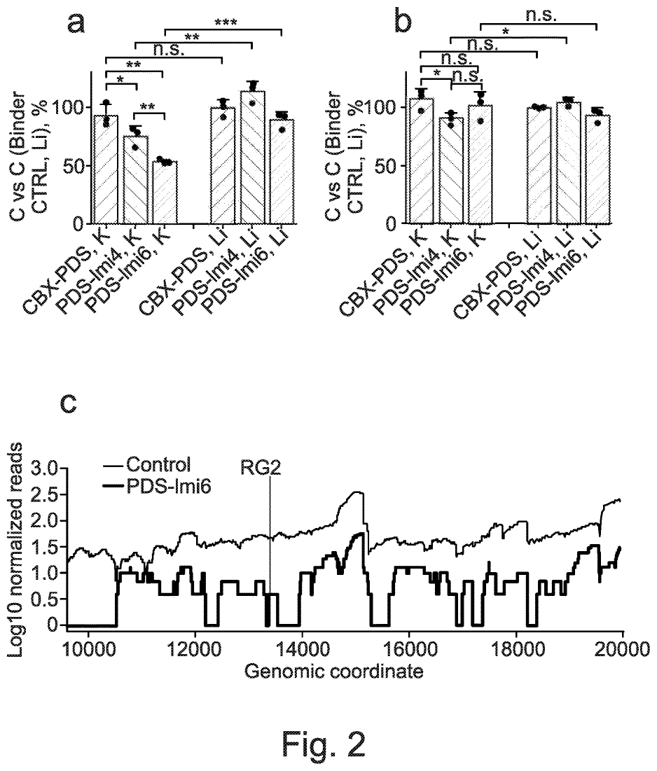

To show that our degraders could selectively degrade G4 structures, we examined their activity in presence of different oligomers and cations. We incubated our degraders with either an RNA oligomer corresponding to a rG4 structure in the 5′ UTR of NRAS mRNA or a mutated version thereof which was not capable of forming rG4. Additionally, we tested our molecules in presence of K⁺ (a promoter of rG4 formation) and Li⁺ (known to prevent rG4 formation). For the rG4 competent oligomer, degradation is observed only in the presence of K⁺, not Li⁺ (FIG. 2a).

This suggests that it is not sufficient for an oligomer to have a G-rich sequence to be targeted by these molecules; oligomers must form a G-quadruplex to be degraded. Strikingly, no degradation is observed with the perturbed rG4-incompetent NRAS sequence with either K⁺ or Li⁺, which again shows that these degraders act specifically on rG4 structures (FIG. 2b). Moreover, no degradation was observed with CBX-PDS—a control molecule which contains the rG4 binder moiety PDS but not the degrader—which demonstrates that binding alone is not sufficient and a degrader moiety is necessary to achieve degradation (FIG. 2a, b). Interestingly, we observed that PDS-deg6 (9A), the degrader with a linker of 6 PEG subunits, was degrading RNA faster than PDS-deg4 (9B), with four PEG subunits. This might be a result of an extended reach of the longer linker. Altogether, these experiments demonstrate that our degraders specifically cleave rG4 species but not unfolded RNA regions.

### In Vitro Degradation of SARS-CoV-2 Genomic Material

To provide evidence that rG4 degraders can degrade the genome of SARS-CoV-2 and gain insight into the mechanism of degradation, we have extracted viral RNA from VERO cells infected with SARS-CoV-2 and treated it with PDS-deg6 (9A), then analysed it via direct RNA sequencing. As SARS-CoV-2 genome has several putative rG4s sites (Zhao et al., 2021) and was shown to be tightly packed hence most of it in close proximity to an rG4 (Ziv et al., 2020), we hoped our degrader would induce wide-spread damage. Indeed, we observed substantial degradation across many regions of the genome, showing that our degrader is potent at damaging the genetic material of SARS-CoV-2 (FIG. 2c). This phenomenon suggests that our degraders should be capable of degrading and hence inactivating viral RNAs inside cells.

Anti-viral activity of rG4 degraders in vitro

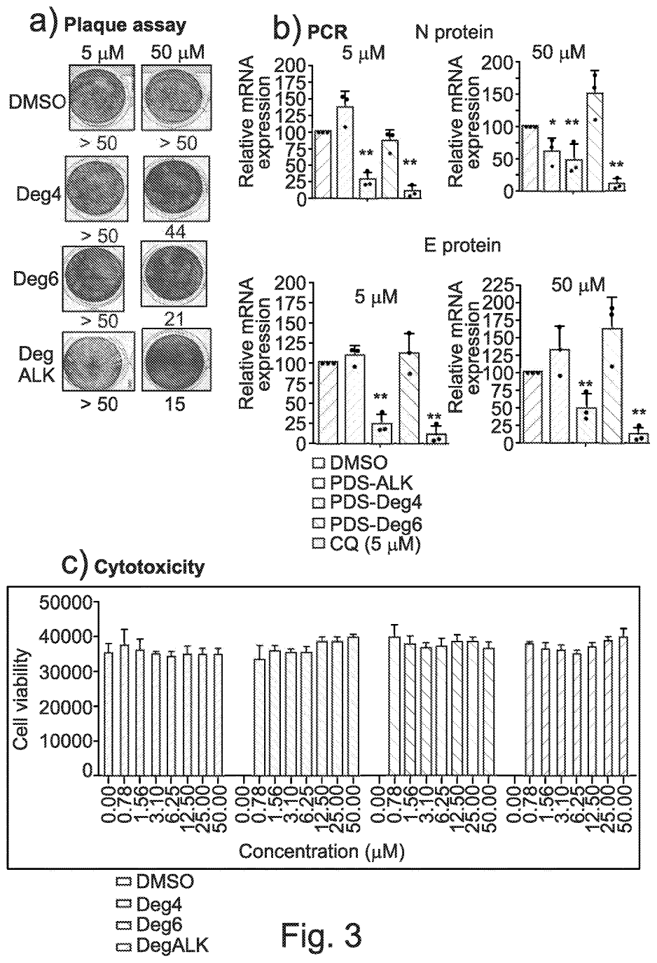

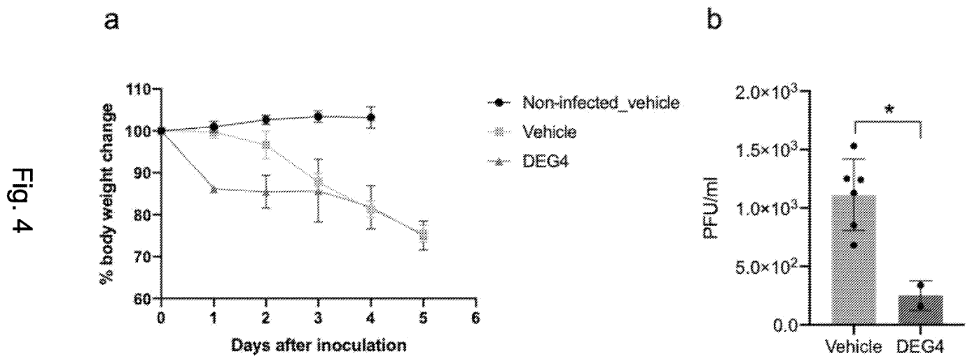

To test the antiviral the antiviral activity of the G4 degraders in vitro, 1 hour prior to infection, cells were incubated with the G4 degraders (PDS-deg6 (9A), PDS-deg4 (9B), and PDS-Alk (8): control molecule without the degrader) at 0.5 μM, 5 μM and 5 μM and chloroquine at 5 μM as a control (FIGS. 3a and 3b). Inhibition of viral growth was be measured by harvesting both the supernatant and cells at 24 hours after infection. Viral growth was assessed by measuring viral loads by plaque assay (in supernatant) and by PCR (in cells). The viability of cells after 24 hours of incubation with increasing concentrations of the G4-degrader (ranging from 0.0 μM to 50 μM) was assessed using a traditional cell viability kit (e.g. CellTiter Blue assay), according to the manufacturer's protocol.

We observed that G4 degraders successfully inhibit viral growth at 5 μM ad 50 μM (FIG. 3). PCR results showed that PDS-deg4 does not seem to inhibit viral replication, while PDS-deg6 (9A) inhibited 70% of viral replication at 5 μM in comparison to DMSO control (FIG. 3b). Importantly, none of the compounds showed cytotoxicity up to 50 μM (FIG. 3c).

### Anti-Viral Activity of rG4 Degraders In Vivo

To assess the in vivo antiviral activity of G4 degraders, transgenic K18hACE2 mice (expressing hACE2 protein) were administered PDS-deg4 (9B) and PDS-deg6 (9A) at intranasally 25 mg/kg 40 minutes before infection, and again at 3 h and 18 h after infection (FIG. 4). Mice were infected with SARS-CoV-2 intranasally (with 2.5-5×10⁴ PFU/mouse in 50 μl of PBS on Day 0) and monitored on a daily basis for body weight, morbidity and mortality (found dead or euthanized in extremis) and clinical signs of infection. On day 5, all mice were sacrificed, and the left lung was collected for viral load quantification by plaque assay. Right lung, heart, liver, kidney and spleen were harvested for histopathological analysis.

Results showed that the administration of PDS-deg6 (9A) at 25 mg/kg was toxic to and these treated mice had to be sacrificed on Day 0. Organs were collected histopathological analysis. Mice administered with PDS-deg4 (9B) showed 10% loss of body weight in the first day after infection (FIG. 4a). However, body weight stabilized between Day 1 and Day 3, after which decreased again at the same rate as vehicle controls. Animals treated with PDS-deg4 (9B) showed a significant decrease in lung viral load (FIG. 4b).

This pilot showed that the administration of G4 degraders leads to decreased viral load in the lungs of SARS-CoV-2 infected k18hACE2 mice.

### Targeting of Pseudoknots and Degradation of RNA

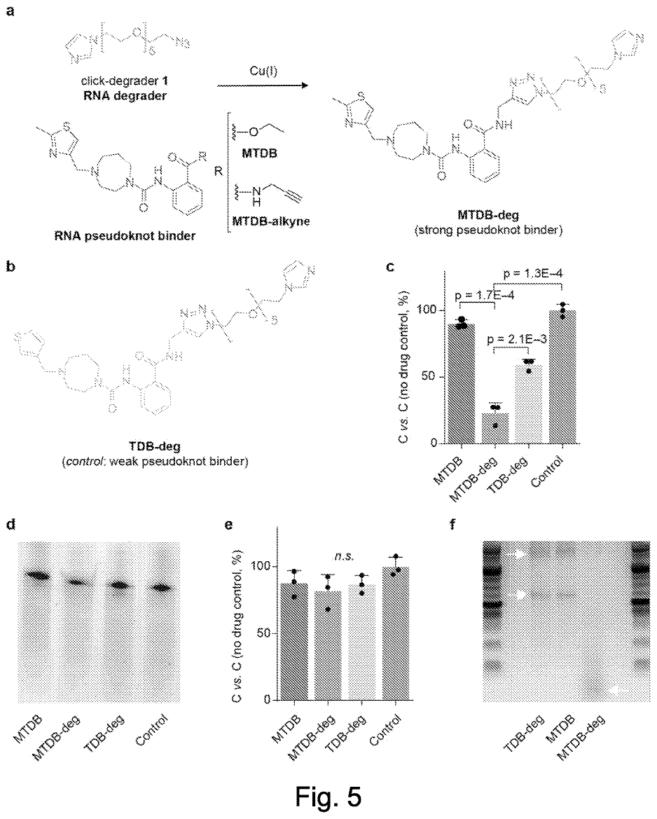

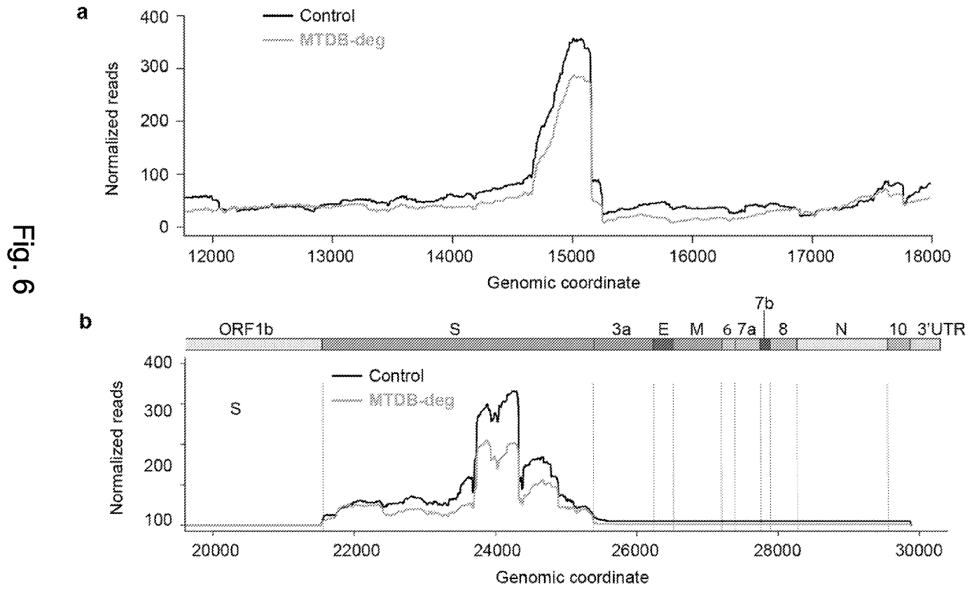

A non-covalent degrader molecule, MTDB-deg (16a), was rationally designed to target an RNA pseudoknot by joining the known pseudoknot binder MTDB with the azido-imidazole 3a (FIG. 5a). MTDB contains an ethyl ester moiety, which was exchanged for an amide to increase stability and for use as a handle for the attachment of the degrader. We used azido-imidazole 3a having a linker consisting of 6 PEG subunits, and which we previously found to be more effective than its shorter counterparts for RNA degradation of alkynyl-tagged RNAs (Mikutis et al., 2020). We chose CuAAC as the reaction to couple binder and degrader fragments because it is robust, easy to carry out, highly modular, and allows us to change the structures of the two fragments without altering the coupling step.

### Selective Degradation of the Three-Stemmed Coronaviral Pseudoknot

To validate our strategy, we tested our pseudoknot-degrader against an RNA 69-er with a sequence that corresponds to the coronaviral pseudoknot and thus is predicted to form it. We incubated the 69-er with MTDB-deg (16a) or one of the two control molecules—MTDB, the parent binder molecule that is not capable of degradation, or TDB-deg (16b), a degrader derived from 2-(4-(thiophen-3-ylmethyl)-[1,4]diazepane-1-carbonyl]-amino)-benzoic acid ethyl ester (TDB), which is closely related to MTDB but has a lower binding affinity towards the pseudoknot (FIG. 5b) (Park et al., 2011). After 3 h incubation with MTDB-deg (16a), the RNA pseudoknot had degraded significantly and just 23% remained intact relative to the non-treated sample, whereas inefficient degradation was seen with TDB-deg (16b) and no degradation was seen with MTDB (FIG. 5c,d). To illustrate that these molecules specifically bind and degrade the pseudoknot, we carried out the same experiment with an oligo similar to the pseudoknot but that harbours a heavily perturbed third stem that prevents it from properly forming the pseudoknot. As expected, none of the molecules affected the oligo stability (FIG. 5e). Thus, MTDB-deg (16a) was shown to bind and degrade the pseudoknot both efficiently and selectively.

To show that MTDB-deg (16a) is functional and can cut full-length coronaviral RNA, we incubated MTDB-deg (16a) and controls MTDB and TDB-deg (16b) with RNA extracted from SARS-CoV-2. Viral RNA was then analysed on agarose gels. We observed degradation only in the lane corresponding to MTDB-deg (16a) (FIG. 5f), further confirming that MTDB-deg (16a) does indeed degrade the native coronaviral pseudoknot, whereas the two control molecules do not, highlighting the specificity of the approach.

### Specificity of Coronaviral Pseudoknot Degradation by MTDB-Deg

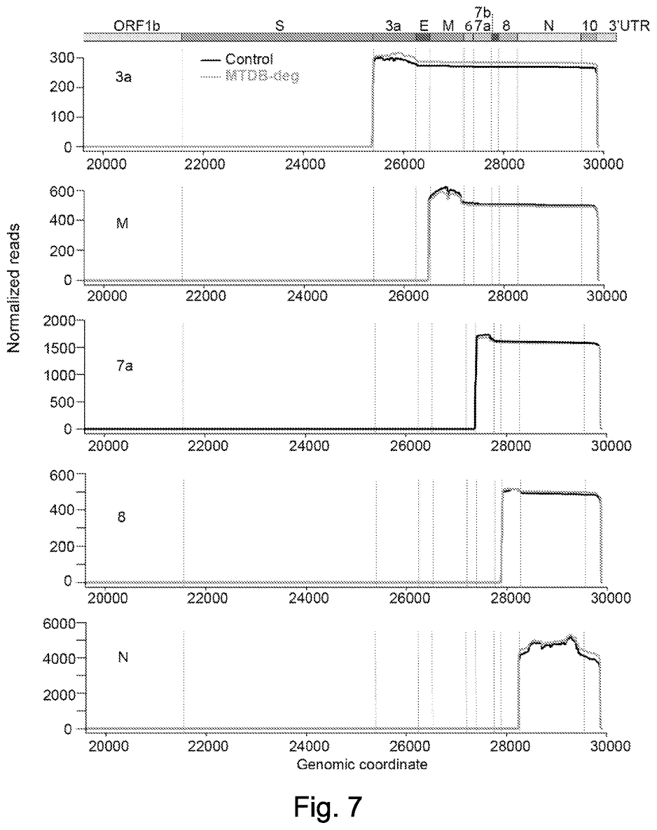

To further prove that MTDB-deg (16a) cuts the viral RNA, and to get a more precise picture of where the cut occurs, we analyzed the cut genomic RNA (gRNA) by direct RNA Nanopore sequencing. As expected, the region around the pseudoknot was affected the most (FIG. 6a). Interestingly, the pseudoknot flanks were more degraded than the pseudoknot itself. Indeed, a study on SARS-Cov-2 RNA interactome found that the region around the frameshifting element forms extensive short- and long-range interactions with the neighboring ORF1a and especially ORF1b; it is likely that the proximity of these elements to the pseudoknot enable MTDB-deg (16a) to efficiently cut them (FIG. 6a) (Ziv, et al., 2020). Interestingly, the only other structural element that was affected by the molecule was the S gene, which was shown to form long-range interactions with the ORF1b (Ziv, et al., 2020) and therefore is expected to be within reach of the degrader (FIG. 6b). Strikingly, none of the other sub-genomic regions were affected, which provides strong evidence for the specificity of MTDB-deg (16a) (FIG. 7). The above results provide strong proof of principle that MTDB-deg (16a) is a fully functional and selective degrader of the SARS-Cov-2 pseudoknot and its direct RNA-RNA interactome (Ziv, et al., 2020).

### Efficiency and Specificity of Pseudoknot Degradation in SARS-CoV-2 Infected Cells

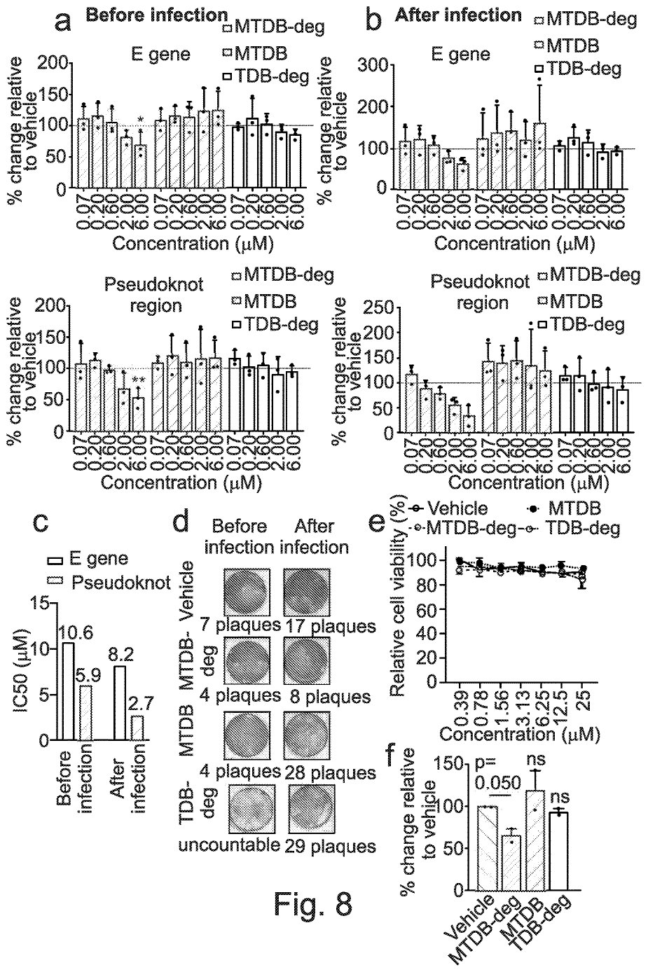

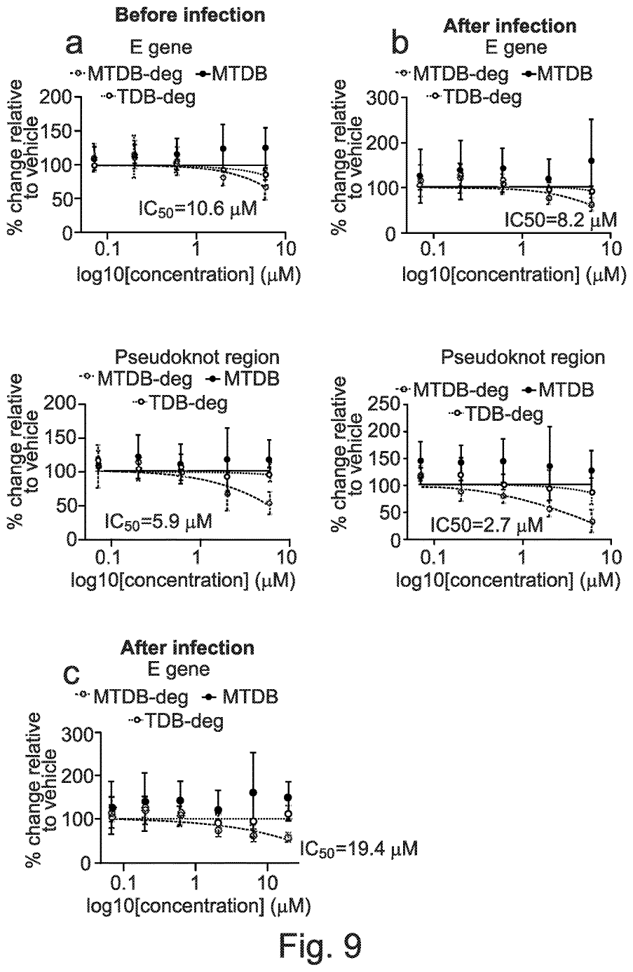

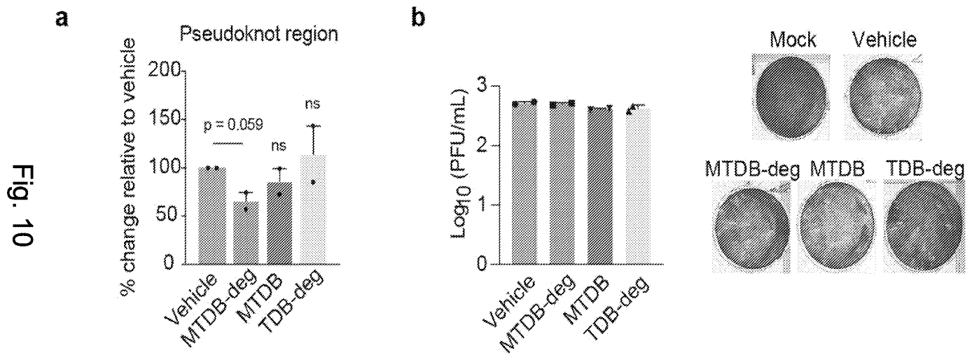

Having demonstrated the efficiency of MTDB-deg (16a) against the coronaviral pseudoknot in vitro, we investigated whether it could degrade the genome of SARS-CoV-2 in infected cells and thus prevent viral replication. We performed in vitro drug assays in which we measured SARS-CoV-2 replication on Vero CCL-81 cells. We observed that low-micromolar concentrations of MTDB-deg (16a) exhibited marked antiviral effects (FIG. 8a-c), with a significant reduction of coronaviral RNA in cells treated before (FIG. 8a; FIG. 9a) or after (FIG. 8b; FIG. 9b) infection. These results were supported by results from plaque assays (FIG. 8d). Importantly, control molecules MTDB and TDB-deg (16b) did not exhibit an antiviral effect, despite MTDB being known to disrupt frameshifting in SARS-CoV-2 (Kelly et al., 2020). Additionally, we found that none of the compounds were cytotoxic to host cells, which indicates that the observed effect on viral replication was a result of the specific antiviral activity of the compound (FIG. 8e). Curiously, the degrader was less active at concentrations higher than 6 μM (FIG. 9c), although no colloidal aggregation that could justify these readouts was seen in dynamic light scattering screens. Furthermore, the ability of the virus to recover from 24 h drug exposure was compromised in MTDB-deg (16a) treated samples, but not in samples treated with the control molecules MTDB and TDB-deg (16b) (FIG. 8f; FIG. 10a). Finally, no virucidal effect was observed when cell free virus was incubated with MTDB-deg (16a), MTDB or TDB-deg (16b), suggesting that the antiviral activity of MTDB-deg (16a) is mediated by direct inhibition of virus replication in host cells (FIG. 10b). Overall, the antiviral drug assays show that MTDB-degrader (16a) is an efficient antiviral agent against SARS-CoV-2 and is specific against coronaviral three-stemmed pseudoknots with irreversible impact.

### Anti-Viral Activity of Pseudoknot Degraders In Vivo

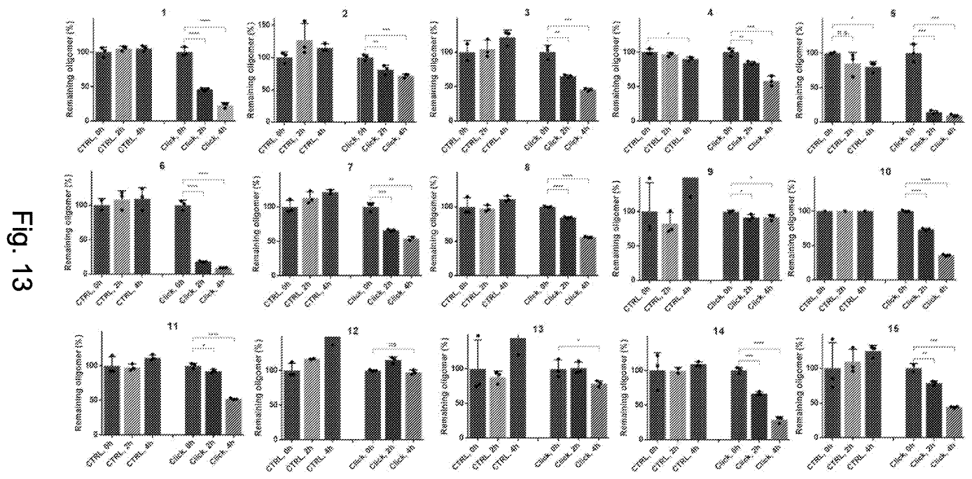

A SARS-CoV-2 mouse model of infection (transgenic K18-hACE2 mice) was used to determine the in vivo antiviral activity of the MTDB-deg 16a (FIG. 12a). Animals administered with MTDB-deg 16a (at 25 mg/kg) showed a significant reduction of lung viral load relative to the vehicle control group by plaque assay (FIG. 12b). Additionally, we investigated the in vivo antiviral potential of either MTDB-deg 16a or vehicle treatments using proteins extracted from lungs of K18-hACE2 transgenic mice on day 3 or day 6 post-infection. Reassuringly, we observed that at both timepoints of infection, the MTDB-deg 16a treated cohort showed a strong reduction in the phosphorylated levels of p38 (FIG. 12c), an important biomarker of SARS-CoV-2 infection and replication.

### Targeting of a Bacterial Ribosome

Three degraders were rationally designed to target the bacterial ribosome by joining the known ribosomal RNA binder chloramphenicol with azido-imidazoles of different lengths (18a, 18b, 18c). The chloramphenicol binder site was produced by peptide coupling of propargylic acid to (1R,2R)-(−)-2-Amino-1-(4-nitrophenyl)-1,3-propanediol using HATU and DIPEA, and followed by the coupling step using copper click chemistry. The copper-induced azide-alkyne cycloaddition (CuAAC) used to join the two components tolerates a vast array of substrates and results in triazole, a bioisostere of an amide and a moiety well-tolerated in biological systems. The three ribosomal degraders and a binding control were tested in vitro and in cellular systems.

In Vitro Degradation of E. coli Ribozyme

The degradation activity was measured in an in vitro assay, targeting the E. coli ribosome. 200 μM ribozyme were incubated with degrader in different concentration, ranging from 15 mM to 0.47 mM, for 18 hours at 37° C. Evaluation was made by agarose gel analysis.

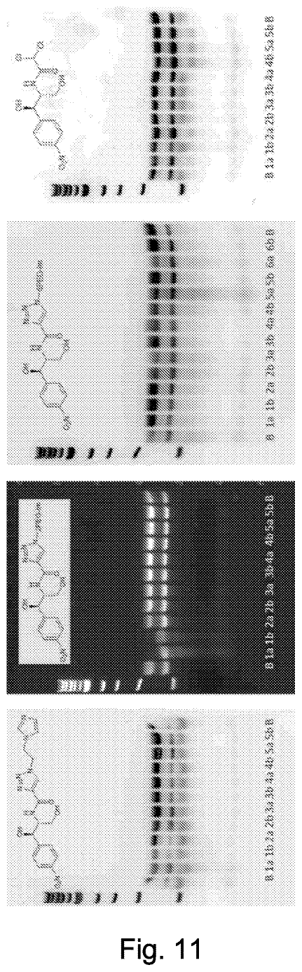

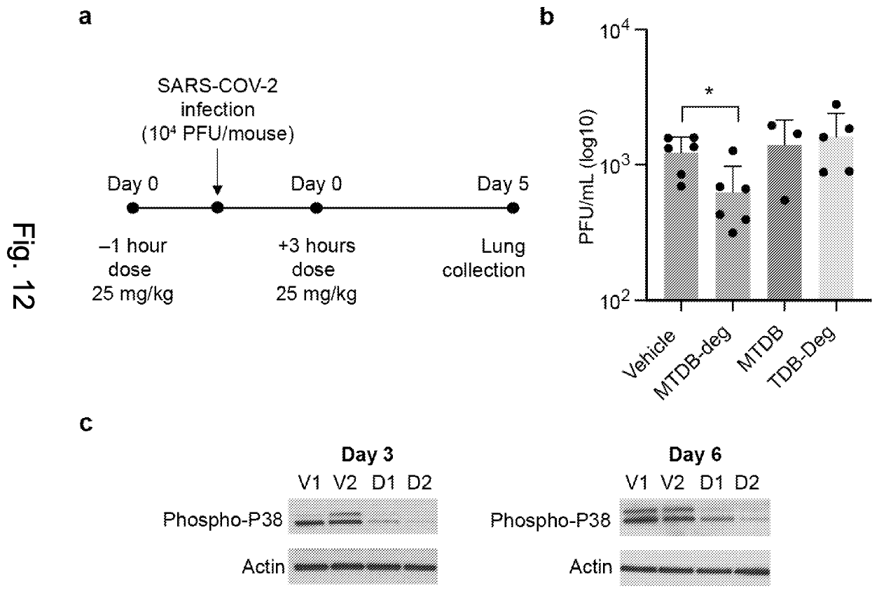

Best results were obtained using the PEG-2 linker (18b), where ribosome degradation could be observed at a concentration of 15 mM (FIG. 11).

### Illustrative Example 2

### Studies to Show the Nucleic Acid Degradation Activity of New Cleavage Groups

Targeted degradation forms part of the cutting edge in drug development. The destruction of ribonucleic acid (RNA) strands in living systems is critical to effective biological function in an organism. Crucial to a broad range of functions, RNAs represent important targets for disruption of disease. Established methods for targeted degradation utilise cellular co-factors, complicating their potential deployment. Additionally, the specificity of this approach carries an inflexibility in the compounds that can be used.

We recently described the development of a strategy that utilises small molecules to degrade specific RNA [Mikutis, S., et al.]. An azide appended to a basic degrader cleavage group (“warhead”) was shown to degrade RNA propargylated at adenosine bases by hijacking of methyltransferase enzymes. By copper-promoted “click” reaction, the degrader was attached to the methylated RNA transcripts, which then underwent proximity-driven cleavage at nearby sites. Two mechanisms were found to be in operation. The first, determined by in vitro pH control, we suspected to be a base-mediated deprotonation of the 2′-O position on the ribose scaffold. The second mechanism, we believed to be mediated by transition metals, based on chelation controls. These findings suggested that our strategy could constitute a powerful general platform for degrading a vast range of RNA targets.

In 2019, a respiratory illness changed the way life is lived. The virus that causes the illness known as COVID-19, SARS-CoV-2, has precipitated our observed need to develop medicines capable of targeting and destroying viral machinery. Beyond covalent modifications, the secondary and tertiary structure of RNA complexes can also have a pathological effect and therefore be exploited as a focus for therapy. After our initial report on chemical degraders, and as shown in Illustrative Example 1 herein, we developed a non-covalent strategy for the targeting of G-quadruplexes: tertiary structures implicated in multiple diseases. The genome of SARS-CoV-2 contains four putative G-Quadruplex sites [Zhao, C., et al.].

Although imidazole, the original bioinspired cleavage group (warhead) we selected for the meClick-seq method, demonstrated itself to be sufficient to prove the basis of our strategy, its simplicity carries with it an inherent lack of tuneability for efficacy and DMPK properties with a view to human treatment. Furthermore, because of the difference in topology for each new RNA binder when complexed, degrader efficiency may vary from target to target. As such, we sought to generate a new library of click degraders to expand the pharmaceutical potential of our technology.

### Results

Disclosed herein is a library of chemical degraders. Directed by theoretical predictions of basicity, endeavors were guided towards degraders with a pKa close in value to the physiological pH 7.4, in the range of 6.2-8.6. Such a pKa would allow the base to be significantly deprotonated under physiological conditions and thus retain its basic character (Scheme A). We also investigated degrader cleavage groups with an ability to strongly bind transition metals (cleavage groups 5, 7). A broad range of degraders were assessed, both to provide enhanced potency and to probe our mechanistic hypothesis (Cleavage Groups 2, 9, 11, 14).

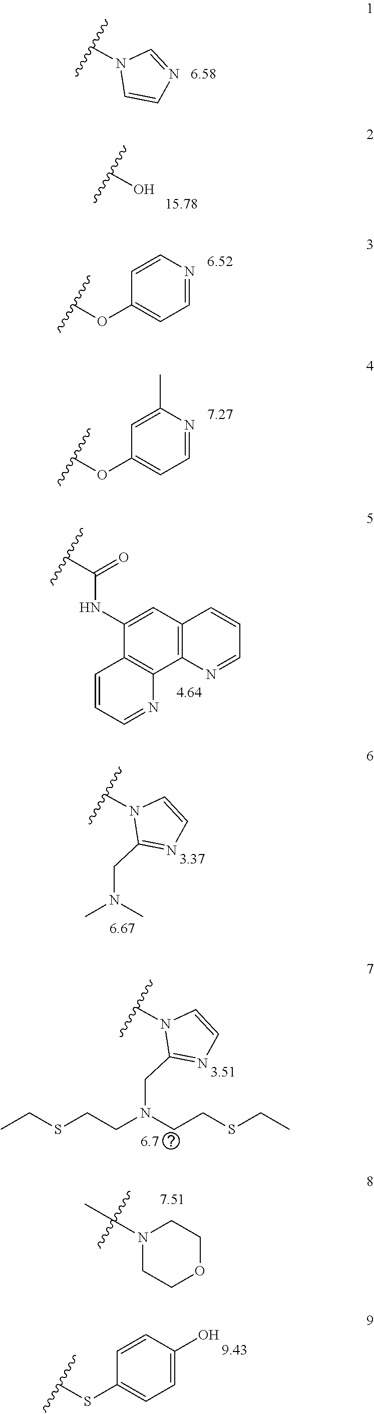

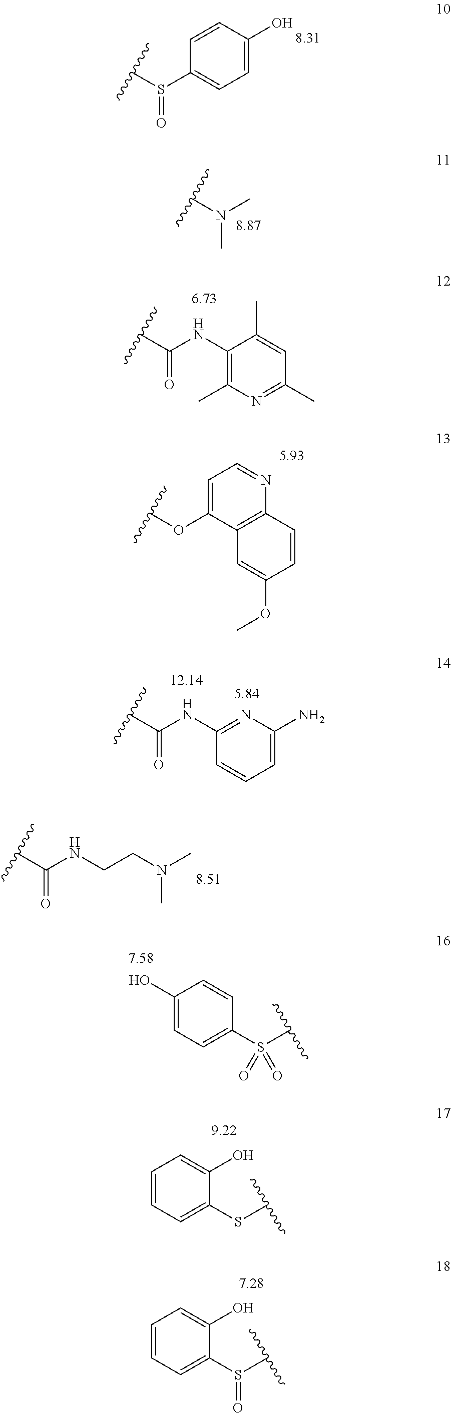

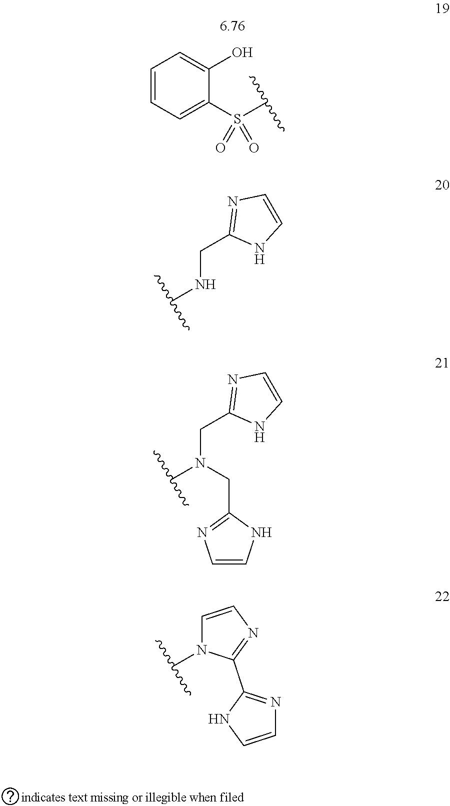

Numbers specify pKa values of particular functional groups in the range of 0-16, as predicted in silico using MarvinView module in KNIME 3.6.1 environment, part of ChemAxon/Infocom Marvin Extensions 3.6.0 package. All degraders were prepared as warhead-hexaethylene glycol-azide constructs.

For assessment of efficacy of degraders, an in vitro assay featuring direct functionalisation of RNA oligonucleotide with a degrader warhead was carried out.

Briefly, an alkyne-tagged RNA oligonucleotide in a HEPES pH 7.4 buffer is reacted with a degrader warhead-hexaethylene glycol-azide construct using a CuAAC (copper-catalysed azide alkyne cycloaddition) reaction. The mixture is then incubated at 37° C. for 0-4 hours, followed by quenching of copper and storing the reaction mixture at 4° C. This mixture was then analysed via LC-MS (liquid chromatography-mass spectrometry). Mass spectrometry signals corresponding to the oligonucleotide-degrader construct are then integrated, various timepoints are compared to the signal at t=0 to estimate degradation (FIG. 2).

As a counter-assay to evaluate specificity of the degraders, a non-alkyne-functionalised oligomer is treated with the degrader and CuAAC components under identical conditions. This oligonucleotide could not be covalently functionalised with an azide thus all the degradation observed in this case would arise from non-specific interactions between degrader warhead and the RNA oligonucleotide, whereas in cases where no such degradation was observed the degradation of covalently functionalised oligonucleotide must be a specific result of induced proximity.

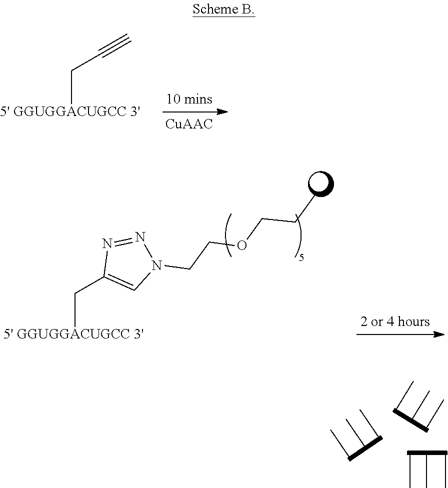

A scheme outlining the in vitro assay for degrader warhead efficiency evaluation. CuAAC is carried out on the alkyne-functionalised oligonucleotide using degrader warhead-hexaethylene glycol-azide constructs to install the degrader cleavage group on the oligonucleotide, linked via triazole and hexaethylene glycol linkage. The functionalised oligonucleotide is either analysed immediately to obtain a t=0 reading or analysed after incubating the mixture at 37° C. for 2 or 4 hours to evaluate degradation at these timepoints. Analysis is carried out via liquid chromatography-mass spectrometry.

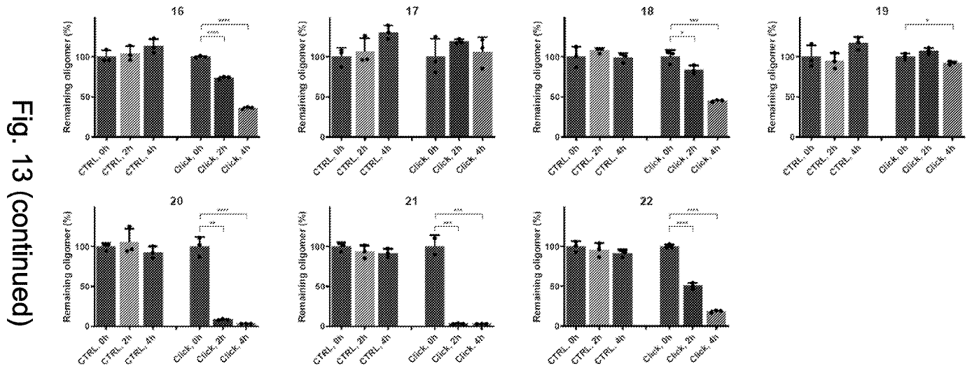

The degrader cleavage groups tested exhibited varying activities and provided insight on design of RNA degrader cleavage groups (FIG. 13). We found that even degraders with a minimalistic cleavage group can carry out degradation (2, 11). The most likely explanation is the ability of the linker to bind copper and bring it to close proximity of the nucleic acid, as absence of degradation on the CTRL strand does indeed suggest that this effect is proximity induced. Curiously, the warhead predicted to be positively charged under conditions of the assay (11) induced a more pronounced degradation than the uncharged minimalistic warhead (2). This might be the case due to the positive charged amino group interacting with the negatively-charged phosphodiester backbone thus anchoring the linker to close proximity of it, perhaps facilitating a hydrogen bond. It is noted that cleavage groups 9 and 10 have very similar structures but 10 is a much more efficient degrader. Without wishing to be bound by any particular theory, it is hypothesised that this can be explained by it having a predicted pKa value close to 7.4 (8.31) and 9 being more basic (predicted pKa 9.43) thus being fully protonated under these conditions and unable to act as a base. Interesting, all cleavage groups except 4 and 5 did not induce any damage on CTRL strand, suggesting that they degrade RNA selectively, only when brought to close proximity of RNA. Phenanthroline-based degrader warhead 5 is a well-known nucleic acid intercalator, which explains its ability to degrade RNA in a non-selective manner [Sigman, D. S., et al.].

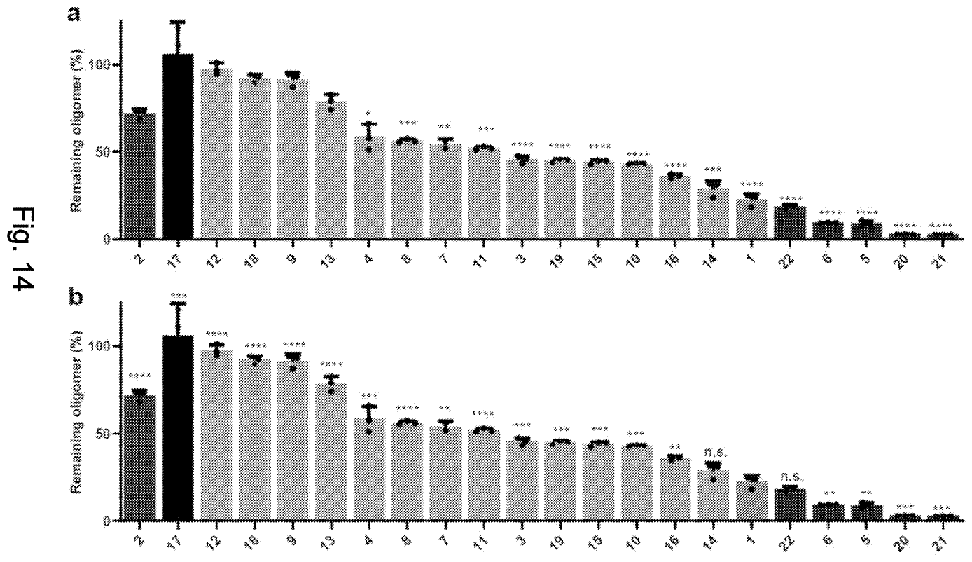

The efficiencies of the 15 degrader cleavage groups were then compared to the warheadless linker (degrader 2) as well as the imidazole-based degrader 1 (FIG. 14). Interestingly, we found that three of the degrader cleavage groups were less efficient degraders than the linker itself; this might imply that these moieties do not facilitate degradation and act as steric blockers, preventing access of the copper-binding linker to the RNA oligonucleotide. Conversely, we found that two of the degraders were significantly more potent than the imidazole degrader 1. One of these degraders, phenanthroline degrader 5, was found to degrade the control RNA strand as well, although to a much lower extent than the functionalised strand, as discussed previously. However, degrader warhead 6 did not exhibit non-specific cleavage and degraded exclusively the covalently functionalised RNA. We found that further eight degraders exhibited intermediate potency—they were more potent than the linker itself but not as potent as imidazole head, which is not surprising given imidazole's prevalence in natural RNA degradation systems.

A library of novel rationally-designed RNA degrader cleavage groups has been prepared and their efficiencies have been compared to a previously described imidazole warhead 1 as well as warheadless hexaethylene glycol linker in an in vitro RNA degradation assay. These degraders exhibited a wide range of potencies. The principles described herein may be used to discover new, potentially superior RNA degrader cleavage groups.

### Experimental

### In Vitro Degrader Warhead Direct Functionalisation-Degrader Efficiency Evaluation Reactions

CuSO₄ (final concentration 1.0 mM), THPTA (3.0 mM), degrader warhead-hexaethylene glycol-azide construct (2.0 mM) and the RNA oligo (200 μM) were added to pH 7.5 buffer supplemented with 10 mM MgCl₂ and 100 mM KCl. CuAAC was initiated by adding NaAsc (50 mM). The reaction mixture was then incubated at 37° C. for 10 minutes. Reaction was quenched either immediately or after a further 2 or 4 hour incubation at 37° C. After quenching the reaction mixtures were analysed using LC-MS. MS signal corresponding to initial degrader-functionalised RNA concentration was estimated from reactions quenched immediately after 10 minute functionalisation, by integrating 3 or 4 m/z intensities corresponding to appropriate RNA species. MS signal corresponding to 2 or 4 hour degradation was compared to the t=0 signal.

### Chemical Synthesis

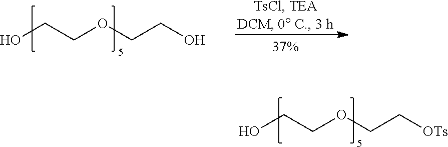

### 17-hydroxy-3,6,9,12,15-pentaoxaheptadecyl 4-methylbenzenesulfonate

Hexaethylene glycol (3.0 g, 11 mmol) was dissolved in DCM and cooled to 0° C., followed by addition of p-toluenesulfonyl chloride (2.2 g, 12 mmol) and TEA (2.1 g, 2.9 ml, 21 mmol). The solution was stirred for 3 h at 0° C. and 30 min at room temperature and quenched with H₂O (20 ml). Organic solvents and volatiles were removed in vacuo, followed by silica column purification (dry loading, gradient EtOAc with 5% MeOH to EtOAc with 10% MeOH). The product was obtained as a colourless oil (1.72 g, 3.9 mmol, 37%).

¹H NMR (400 MHz, CDCl3) δ 7.79 (d, J=7.9 Hz, 2H), 7.33 (d, J=7.9 Hz, 2H), 4.15 (t, J=4.4 Hz, 2H), 3.72-3.58 (m, 22H), 2.52 (s, 1H), 2.44 (s, 3H). ¹³C NMR (101 MHz, CDCl3) δ 144.89, 133.19, 129.94, 128.10, 72.70, 70.84, 70.73, 70.68, 70.64, 70.41, 69.39, 68.81, 61.84, 21.75. HRMS m/z: calculated m/z for [C¹⁹H³²O⁹S]⁺ 437.1854; observed 437.1845.

### 17-azido-3,6,9,12,15-pentaoxaheptadecan-1-ol (1)

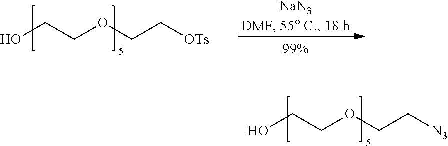

17-hydroxy-3,6,9,12,15-pentaoxaheptadecyl 4-methylbenzenesulfonate (524 mg, 1.2 mmol) was dissolved in anhydrous DMF (3 ml). Sodium azide (112 mg, 1.7 mmol) was added, the mixture was placed under N₂ and stirred for 18 h at 55° C. Solvent was removed in vacuo. To remove traces of DMF the residue was co-evaporated successively with portions of Toluene. The resulting residue was dissolved in DCM and filtered. The resulting residue was purified on a column (gradient, 9:1 DCM:MeOH). The product was obtained as a pale-yellow oil (200 mg, 0.64 mmol, 54%).

¹H NMR (400 MHz, CDCl3) δ 3.72-3.59 (m, 22H), 3.38 (t, J=5.1 Hz, 2H), 2.54 (s, 1H). ¹³C NMR (101 MHz, CDCl3) δ 72.75, 70.78, 70.75, 70.68, 70.64, 70.41, 70.15, 61.85, 50.82. HRMS m/z: calculated m/z for [C₁₂H₂₅N₃O₆Na]+ 330.1652; observed 330.1641.

### 1-(17-azido-3,6,9,12,15-pentaoxaheptadecyl)-1H-imidazole (2)

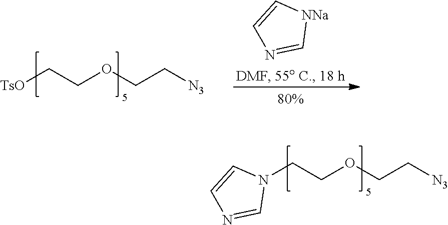

Imidazole (44 mg, 0.65 mmol) and NaH (60% dispersion in mineral oil, 26 mg, 0.65 mmol) were suspended in anhydrous DMF (2 ml) at 0° C. The mixture was placed under N₂ atmosphere, allowed to warm to room temperature and stirred for 30 min. 4 (250 mg, 0.54 mmol) was dissolved in anhydrous DMF (3 ml) and the resulting solution was added to the first mixture. It was then stirred for 20 h at 55° C. Solvent was then removed in vacuo and the resulting residue was purified via flash chromatography (dry loading, gradient EtOAC to 9:1 EtOAc:MeOH). The product was obtained as a colourless oil (154 mg, 0.43 mmol, 80%).

¹H NMR (400 MHz, CDCl₃) δ_{H} 7.52 (s, 1H), 7.02 (s, 1H), 6.98 (s, 1H), 4.09 (t, 2H), 3.72 (t, 2H), 3.55-3.78 (18H, PEG), 3.36 (t, 2H). ¹³C NMR (100 MHz, CDCl₃) δ_{C} 137.6, 129.3, 119.4, 70.6-70.7 (Multiple PEG peaks), 70.0, 50.7, 47.0. HRMS m/z: [M+H]⁺: calculated m/z for [C₁₅H₂₈N₅O₅]⁺ 358.2090; observed 358.2084.

### 4-((17-azido-3,6,9,12,15-pentaoxaheptadecyl)oxy)-pyridine (3)

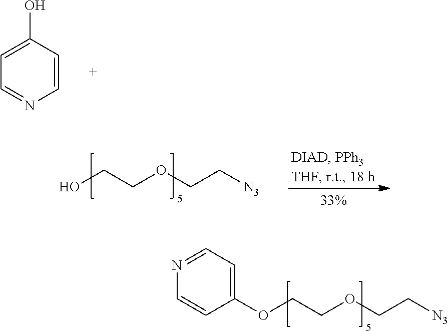

4-hydroxy-pyridine (77.4 ma. 810 mmol). hexaethylene glycol azide (200 mg, 650 umol) and triphenylphosphine (214 mg, 810 umol) were dissolved in dry THF, degassed via freeze-pump-thaw (3 cycles) and put under 0° C. and argon atmosphere. DIAD (165 mg, 810 umol) was then added, mixture was allowed to warm to room temperature and stirred overnight. 3 g/l solution of NH₄Cl (20 ml) was then added to quench the reaction and the resulting mixture was washed with DCM (3×10 ml). 1% NaOH solution (10 ml) was added to the aqueous fraction and the product was extracted with DCM (9×20 ml). Organic solvent was removed, the resulting yellow oil was purified on a silica column (gradient, AcOEt to AcOEt with 10% MeOH and 1% TEA. The title compound was obtained as a yellow oil (82 mg, 0.21 mmol, 33%).

¹H NMR (400 MHz, CDCl₃): δ 7.34 (d, 2H), 6.33 (d, 2H), 3.90 (tr, 2H), 3.74 (tr, 2H), 3.55-3.66 (m, 18H), 3.36 (tr, 2H), 2.43 (brs, 6H); ¹³C NMR (100 MHz, CDCl₃): δ_{C} 179.0, 140.3, 118.5, 70.8, 70.5-70.7 (Multiple PEG peaks), 70.1, 70.0, 56.6, 50.7; HRMS m/z: [M+H]⁺: calculated m/z for [C₁₇H₂₈N₄O₆]⁺ calculated m/z for 385.2082; observed 385.2079.

### 4-((17-azido-3,6,9,12,15-pentaoxaheptadecyl)oxy)-2-methylpyridine (4)

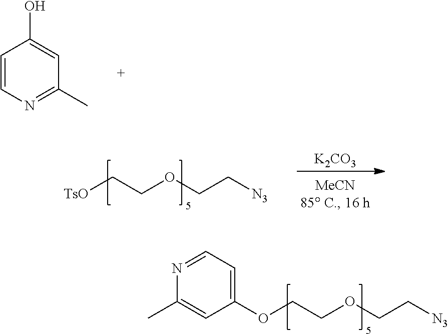

A mixture of 2-methylpyridin-4-ol (10.9 mg, 0.10 mmol, 1.0 equiv.), 17-azido-3,6,9,12,15-pentaoxaheptadecyl 4-methylbenzenesulfonate (47.3 g, 0.10 mmol, 1.0 equiv.) and K₂CO₃ (26.6 mg, 0.20 mmol, 2.0 equiv.) in acetonitrile (2 mL) was stirred under nitrogen in a sealed tube at 85° C. for 16 h. The reaction was cooled, ethyl acetate (20 mL) was added and the mixture was filtered through Celite and concentrated in vacuo. The resultant residue was purified on silica (eluting with 80% acetone/hexanes) to yield the title compound as a colourless oil (28.1 mg, 0.071 mmol, 71%).

¹H NMR (400 MHz, CDCl₃) δ 8.29 (d, J=5.8 Hz, 1H), 6.68 (d, J=2.4 Hz, 1H), 6.64 (dd, J=5.8, 2.4 Hz, 1H), 4.15-4.13 (m, 2H), 3.86-3.84 (m, 2H), 3.71-3.69 (m, 2H), 3.67-3.64 (m, 16H), 3.37 (t, J=5.1 Hz, 2H), 2.49 (s, 3H); ¹³C NMR (126 MHz, CDCl₃) δ 165.3, 160.1, 150.4, 109.6, 107.8, 71.0, 70.8, 70.8, 70.8, 70.7, 70.7, 70.7, 70.2, 69.5, 67.2. HRMS m/z: [M+H]⁺ calculated for [C₁₈H₃₁N₄O₆]⁺ 399.2238, found: 399.2243.

### 20-azido-N-(1,10-phenanthrolin-5-yl)-3,6,9,12,15,18-hexaoxaicosanamide (5)

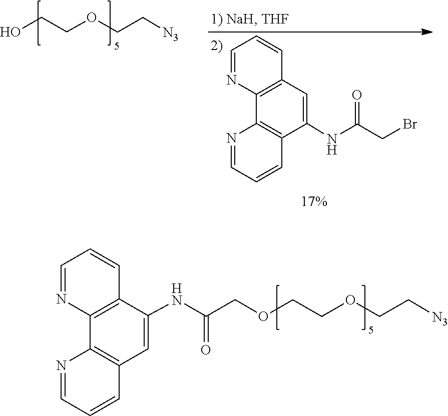

Hexaethylene glycol azide (48.7 mg, 320 umol) was dissolved in THE (1.5 ml) and put under argon atmosphere at 0° C. NaH (60% in mineral oils, 6.3 mg, 320 umol) was then added, mixture was then stirred for 30 minutes at 0° C., followed by 30 minutes at room temperature. 2-bromo-N-(1,10-phenanthrolin-5-yl)acetamide (49.7 mg, 160 umol) was then added, and the mixture was stirred at room temperature overnight. Solvent was then removed in vacuo; the resulting red oil was subjected to HPLC purification and lyophilisation. The title compound was obtained as a red oil (18.3 mg, 33.7 umol, 17%).

¹H NMR (400 MHz, CDCl₃): δ 8.92 (br d, 1H), 8.87 (br d, 1H), 8.20 (d, 2H), 8.15 (d, 2H), 7.65 (m, 2H), 7.60 (m, 2H), 4.36 (br s, 2H), 3.92 (m, 2H), 3.82 (m, 2H), 3.71 (m, 2H), 3.68 (m, 2H), 3.59 (m, 2H), 3.52 (m, 2H), 3.43 (m, 4H), 3.32-3.38 (m, 10H). ¹³C NMR (100 MHz, D2O): δ_{C} 172.4, 150.1, 150.0, 144.6, 143.2, 137.0, 131.9, 129.4, 127.8, 124.7, 121.1, 123.7, 122.6, 70.8, 69.9, 69.9, 69.7, 69.6, 69.3-69.5 (Multiple PEG peaks), 69.1, 50.1. HRMS m/z: [M+H]⁺ calculated for [C₂₆H₃₄N₆O₇]⁺ calculated m/z for 543.2562; observed 543.2560. 1-(1-(17-azido-3,6,9,12,15-pentaoxaheptadecyl)-1H-imidazol-2-yl)-N,N-dimethylmethanamine (6)

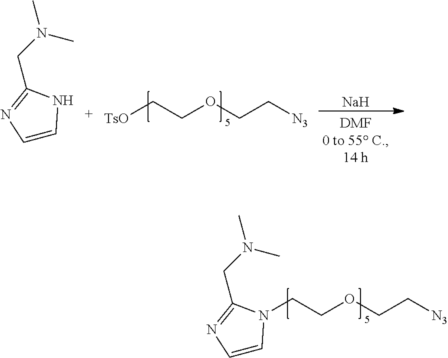

To a solution of 1-(1H-imidazol-2-yl)-N,N-dimethylmethanamine (24.4 mg, 0.195 mmol, 1.2 equiv.) and 17-azido-3,6,9,12,15-pentaoxaheptadecyl 4-methylbenzenesulfonate (75.0 mg, 0.160 mmol, 1.0 equiv.) in DMF (2 mL) was added NaH (5.1 mg, 0.211 mmol, 1.3 equiv.) at 0° C. and then warmed to room temperature and stirred at 55° C. for 14 h. The reaction mixture was subsequently cooled and carefully quenched with H₂O (25 mL) at 0° C. The aqueous layer was then extracted with EA (25 mL×3). The combined organic layer was dried over Mg₂SO₄ and concentrated under reduced pressure. The crude residue was purified on basic alumina, eluting with a gradient of 0% to 30% MeOH/EtOAc to yield the title compound as a colourless oil (35.1 mg, 0.085 mmol, 53%).

¹H NMR (500 MHz, CDCl₃) δ 6.98 (s, 1H), 6.89 (s, 1H), 4.20 (t, J=5.4 Hz, 2H), 3.87 (s, 2H), 3.72 (t, J=5.1 Hz, 2H), 3.67-3.58 (m, 12H), 3.56-3.55 (m, 6H), 3.72 (t, J=5.1 Hz, 1H), 3.51 (s, 2H), 3.37 (t, J=4.9 Hz, 2H), 2.18 (s, 6H); ¹³C NMR (126 MHz, CDCl₃) δ 145.4, 127.1, 121.1, 70.8, 70.8, 70.8, 70.8, 70.7, 70.7, 70.6, 70.1, 56.3, 50.8, 45.9, 45.4. HRMS m/z: [M+H]⁺ calculated for [C₁₈H₃₅N₆O₅]⁺ 415.2663, found: 415.2662.

### N-((1H-imidazol-2-yl)methyl)-2-(ethylthio)-N-(2-(ethylthio)ethyl)ethan-1-amine

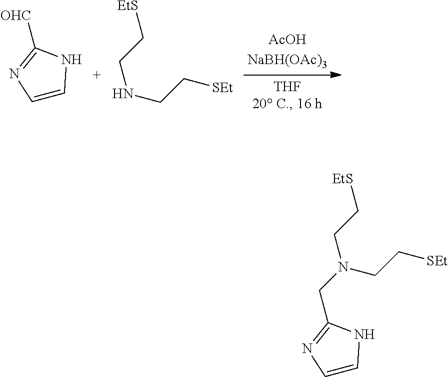

1H-imidazole-2-carbaldehyde (3 mg, 3.67 mmol, 1.1 equiv.) and bis(2-(ethylthio)ethyl)amine (646 mg, 3.34 mmol, 1.0 equiv.) were mixed in THF (12 mL) at rt under N₂. Glacial AcOH (200 mL, 3.34 mmol, 1 equiv.) was added, followed by sodium triacetoxyborohydride (1.06 g, 5.01 mmol, 1.5 equiv.) and the mixture was stirred at 20° C. for 16 h. The reaction mixture was quenched with aqueous saturated NaHCO₃ solution (50 mL), and the product was extracted with EtOAc. The combined organic phases were dried over MgSO₄ and concentrated. The residue was purified on basic alumina, eluting with a gradient of 0% to 70% EtOAc/petroleum ether (40-60 b.p.) to give the title compound as a brown gum (320 mg, 1.17 mmol, 35%).

¹H NMR (700 MHz, CDCl₃) δ 6.98 (s, 2H), 3.80 (s, 2H), 2.76 (t, J=6.7 Hz, 4H), 2.63 (t, J=7.6 Hz, 4H), 2.49 (q, J=7.4 Hz, 4H), 1.22 (t, J=7.4 Hz, 6H); ¹³C NMR (126 MHz, CDC₃) δ 145.0, 121.4, 53.9, 51.7, 29.3, 26.2, 15.0. HRMS m/z: [M+H]⁺ calculated for [C₁₂H₂₄N₃S₂]⁺ 274.1406, found: 274.1409.

### N-((1-(17-azido-3,6,9,12,15-pentaoxaheptadecyl)-1H-imidazol-2-yl)methyl)-2-(ethylthio)-N-(2-(ethylthio)ethyl)ethan-1-amine (7)

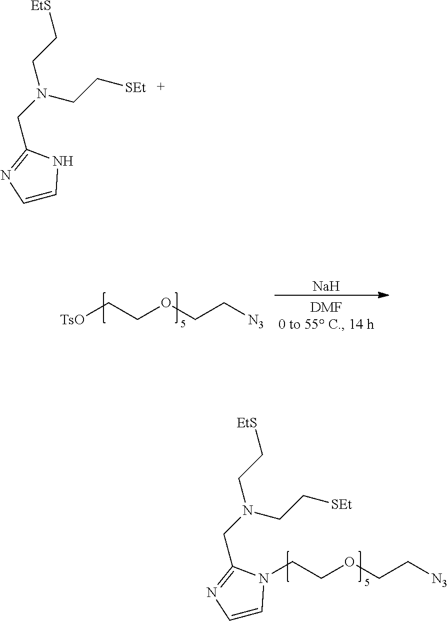

To a solution of N-((1H-imidazol-2-yl)methyl)-2-(ethylthio)-N-(2-(ethylthio)ethyl)ethan-1-amine (78 mg, 0.283 mmol, 1.2 equiv.) in dry DMF (2 mL) was added NaH (12 mg, 0.295 mmol, 1.25 equiv) and the resulting suspension stirred for 30 minutes at 0° C. 17-Azido-3,6,9,12,15-pentaoxaheptadecyl 4-methylbenzenesulfonate (109 mg, 0.236 mmol, 1.0 equiv.) in 1 mL dry DMF was then added at 0° C.. The mixture was warmed to room temperature and stirred at 55° C. for 20 h. The reaction mixture was subsequently cooled and carefully quenched with saturated NaHCO₃ solution (25 mL) at 0° C. The mixture was then extracted with diethyl ether (3×25 mL). The combined organic layer was dried over MgSO₄ and concentrated under reduced pressure. The crude residue was purified on silica, equilibrated with 1% NEt₃/EtOAc. The title product was eluted with a gradient of 0% to 8% MeOH/EtOAc to yield a yellow oil (99 mg, 0.176 mmol, 74%).

¹H NMR (700 MHz, CDCl₃) δ 7.07 (s, 1H), 6.96 (s, 1H), 4.37 (t, J=4.5 Hz, 2H), 3.87 (s, 2H), 3.76 (t, J=5.2 Hz, 2H), 3.68-3.63 (m, 12H), 3.62-3.57 (m, 6H), 3.38 (t, J=5.1 Hz, 2H), 2.72 (t, J=7.2 Hz, 4H), 2.57 (t, J=7.2 Hz, 4H), 2.47 (q, J=7.4 Hz, 4H), 1.21 (t, J=7.4 Hz, 6H); ¹³C NMR (126 MHz, CDCl₃) δ 145.0, 127.2, 121.4, 71.0, 70.9, 70.8, 70.8, 70.8, 70.8, 70.7, 70.2, 53.9, 51.7, 50.8, 46.1, 29.3, 26.2, 15.0. HRMS m/z: [M+H]⁺ calculated for [C₂₄H₄₇N₆O₅S₂]⁺ 563.3044, found: 563.3045.

### 4-(17-azido-3,6,9,12,15-pentaoxaheptadecyl)morpholine (8)

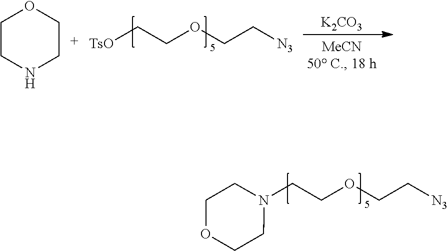

A 4 mL reaction vial was charged with 17-azido-3,6,9,12,15-pentaoxaheptadecyl 4-methylbenzenesulfonate (46.4 mg, 0.10 mmol, 1.0 equiv.) and K₂CO₃ (26.6 mg, 0.20 mmol, 2.0 equiv.), sealed and set under nitrogen. Dry acetonitrile (2 mL) was then added, followed by morpholine (12.9 mL, 0.15 mmol, 1.5 equiv.). The sealed tube was then heated at 50° C. for 18 h. The reaction was cooled, ethyl acetate (20 mL) was added, and the mixture was filtered through Celite and concentrated in vacuo. The resultant residue was purified on basic alumina (eluting with 70% ethyl acetate in hexanes) to yield the title compound as a colourless oil (15.5 mg, 0.041 mmol, 41%).

¹H NMR (500 MHz, CDC₃) δ 3.68-3.63 (m, 16H), 3.62-3.60 (m, 2H), 3.57 (t, J=5.9 Hz, 2H), 3.38 (t, J=5.1 Hz, 2H), 2.50 (t, J=5.9 Hz, 2H), 2.25 (s, 6H). ¹³C NMR (126 MHz, CDCl₃) δ 70.8, 70.8, 70.8, 70.7, 70.7, 70.5, 70.2, 69.5, 58.9, 50.8, 46.0. HRMS m/z: [M+H]⁺ calculated for [C₁₆H₃₃N₄O₆]⁺ 377.2395, 377.2399.

### 4-((17-azido-3,6,9,12,15-pentaoxaheptadecyl)thio)phenol (9)

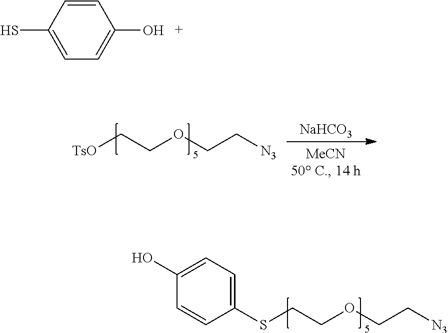

A 4 mL reaction vial was charged with 4-mercaptophenol (18.9 mg, 0.15 mmol, 1.0 equiv.) and NaHCO₃ (30.0 mg, 0.30 mmol, 2.0 equiv.) and set under nitrogen. Dry acetonitrile (1 mL) was then added and the mixture was stirred at 25° C. for 1 h. A solution of 17-azido-3,6,9,12,15-pentaoxaheptadecyl 4-methylbenzenesulfonate (71.9 g, 0.156 mmol, 1.04 equiv.) in dry acetonitrile (2 mL) was added and the sealed tube was heated at 50° C. for 16 h. The reaction was cooled, ethyl acetate (20 mL) was added and the mixture was filtered through Celite and concentrated in vacuo. The resultant residue was purified on silica (eluting with 1% MeOH/CH₂Cl₂) to yield the title compound as a colourless oil (39.9 mg, 0.096 mmol, 64%).

¹H NMR (500 MHz, CDC₃) δ 7.35-7.32 (m, 2H), 6.81-6.78 (m, 2H), 3.68-3.55 (m, 20H), 3.38 (t, J=5.2 Hz, 2H), 2.97 (t, J=6.8 Hz, 2H); ¹³C NMR (126 MHz, CDC₃) δ 155.8, 134.1, 116.3, 70.9, 70.9, 70.8, 70.7, 70.7, 70.6, 70.5, 70.3, 70.2, 50.8, 35.3. HRMS m/z: [M−H]⁺ calculated for [C₁₈H₂₈N₃O₆S]⁻ 414.1704, found: 414.1705.

### 4-((17-azido-3,6,9,12,15-pentaoxaheptadecyl)sulfinyl)phenol (10)

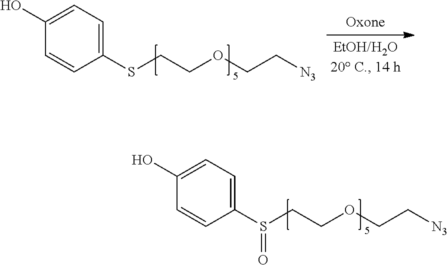

To a solution of 4-((17-azido-3,6,9,12,15-pentaoxaheptadecyl)thio)phenol (28 mg, 0.066 mmol, 1 equiv.) in 1:1 H₂O/EtOH (4 mL) was added oxone (25 mg, 0.165 mmol, 2.5 equiv.). The resultant suspension was stirred at 20° C. for 14 h. Distilled water was added (50 mL) and the pH was adjusted to 5.0 using 1 M HCl. The aqueous solution was extracted with CHCl₃ (4×50 mL) and the combined organic phases were dried over anhydrous MgSO₄ and concentrated. The residue was purified on silica, eluting with 9% methanol in dichloromethane to give the title compound as a colourless oil (26.2 mg, 92%, 0.61 mmol).

¹H NMR (500 MHz, CDCl₃) δ 7.50 (d, J=8.7 Hz, 2H), 6.97 (d, J=8.7 Hz, 2H), 3.84-3.80 (m, 1H), 3.69-3.52 (m, 19H), 3.37 (t, J=5.1 Hz, 2H), 3.13-3.08 (m, 1H), 3.01-2.96 (m, 1H); ¹³C NMR (126 MHz, CDCl₃) δ 160.1, 132.6, 126.6, 116.7, 70.8, 70.7, 70.7, 70.7, 70.7, 70.6, 70.1, 64.1, 57.4, 50.7. HRMS m/z: [M−H]⁻ calculated for [C₁₈H₂₈N₃O₇S]⁻ 430.1653, found: 430.1652.

### 17-azido-N,N-dimethyl-3,6,9,12,15-pentaoxaheptadecan-1-amine (11)

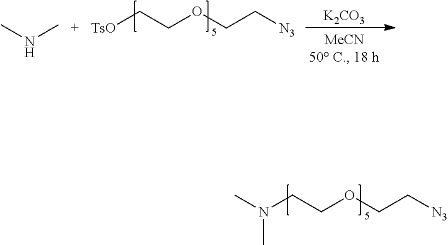

A 4 mL reaction vial was charged with 17-azido-3,6,9,12,15-pentaoxaheptadecyl 4-methylbenzenesulfonate (46.4 mg, 0.10 mmol, 1.0 equiv.) and K₂CO₃ (26.6 mg, 0.20 mmol, 2.0 equiv.), sealed and set under nitrogen. Dry acetonitrile (2 mL) was then added, followed by a 2 M solution of diethylamine in methanol (75 mL, 0.15 mmol, 1.5 equiv.). The sealed tube was then heated at 50° C. for 18 h. The reaction was cooled, ethyl acetate (20 mL) was added and the mixture was filtered through Celite and concentrated in vacuo. The resultant residue was purified on basic alumina (eluting with ethyl acetate) to yield the title compound as a colourless oil (28.1 mg, 0.084 mmol, 84%).

¹H NMR (500 MHz, CDCl₃) δ 3.68-3.63 (m, 16H), 3.62-3.60 (m, 2H), 3.57 (t, J=5.9 Hz, 2H), 3.38 (t, J=5.1 Hz, 2H), 2.50 (t, J=5.9 Hz, 2H), 2.25 (s, 6H). ¹³C NMR (126 MHz, CDCl₃) δ 70.8, 70.8, 70.8, 70.7, 70.7, 70.5, 70.2, 69.5, 58.9, 50.8, 46.0. HRMS m/z: [M+H]⁺ calculated for [C₁₄H₃₁N₄O₅]⁺ 335.2289, found: 335.2285.

### 17-azido-N-(2,4,6-trimethylpyridin-3-yl)-3,6,9,12,15-pentaoxaheptadecanamide (12)

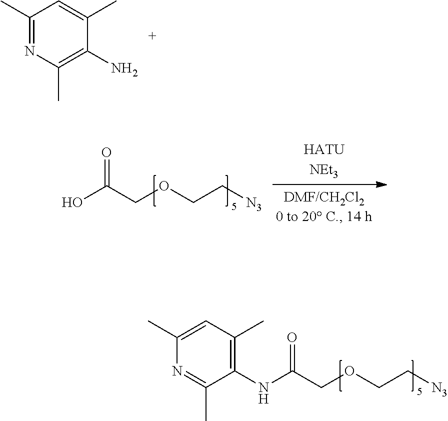

To a solution of 17-azido-3,6,9,12,15-pentaoxaheptadecanoic acid (30 mg, 0.09 mmol, 1 equiv.) in 1 mL of dry CH₂Cl₂ was added HATU (38 mg, 0.1 mmol, 1.1 equiv.), followed by triethylamine (20 mg, 0.2 mmol, 2.2 equiv) dropwise. The solution was cooled to 0° C. under nitrogen atmosphere, incubated for 30 min, then a solution of 1,2-thing (13.6 mg, 0.11 mmol, 1.1 equiv.) in 1 mL dry CH₂Cl₂ was added. The reaction was allowed to warm to 20° C. and stir for 14 hours. The resulting mixture was diluted with CH₂Cl₂ and washed with saturated sodium carbonate solution (3×20 ml). The combined organic phases were dried over MgSO₄, filtered and concentrated under reduced pressure. The residue was purified by chromatography, eluting with 5% methanol in ethyl acetate to yield the title compound as a yellowish oil (5 mg, 11%, 0.011 mmol).

¹H NMR (500 MHz, CDCl₃) δ 9.10 (br. s, 1H), 7.17 (s, 1H), 4.21 (s, 2H), 3.83-3.81 (m, 2H), 3.74-3.72 (m, 2H), 3.67-3.66 (m, 2H), 3.62 (m, 2H), 3.60-3.57 (m, 4H), 3.56-3.54 (m, 2H), 3.51-3.50 (m, 2H), 3.43-3.41 (m, 2H), 3.37-3.35 (m, 2H), 2.68 (s, 3H), 2.64 (s, 3H), 2.37 (s, 3H); HRMS m/z: [M+H]⁺ calculated for [C₂₀H₃₄N₅O₆]440.2504, found: 440.2510.

### 4-((17-azido-3,6,9,12,15-pentaoxaheptadecyl)oxy)-6-methoxyquinoline (13)

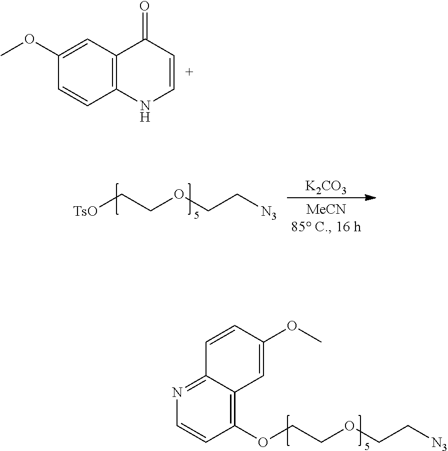

A mixture of 6-methoxyquinolin-4-ol (35.0 mg, 0.20 mmol, 1.0 equiv.), 17-azido-3,6,9,12,15-pentaoxaheptadecyl 4-methylbenzenesulfonate (92.3 g, 0.20 mmol, 1.0 equiv.) and K₂CO₃ (55.3 mg, 0.40 mmol, 2.0 equiv.) in acetonitrile (2 mL) was stirred under nitrogen in a sealed tube at 85° C. for 16 h. The reaction was cooled, ethyl acetate (20 mL) was added and the mixture was filtered through Celite and concentrated in vacuo. The resultant residue was purified on silica (eluting with 50% acetone/hexanes) to yield the title compound as a colourless oil (67.5 mg, 0.145 mmol, 73%).

¹H NMR (500 MHz, CDCl₃) δ 8.62 (d, J=5.5 Hz, 1H), 8.08 (d, J=9.2 Hz, 1H), 7.48 (d, J=2.9 Hz, 1H), 7.39 (dd, J=9.2, 2.9 Hz, 1H), 6.82 (d, J=5.5 Hz, 1H), 4.42 (dd, J=5.6, 4.2 Hz, 2H), 4.03-4.02 (m, 2H), 3.79-3.77 (m, 2H), 3.70-3.68 (m, 2H), 3.66-3.63 (m, 14H), 3.37 (t, J=5.0 Hz, 2H); ¹³C NMR (126 MHz, CDCl₃) δ 161.7, 157.8, 147.3, 140.8, 129.0, 123.0, 122.1, 101.1, 100.1, 71.0, 70.7, 70.7, 70.7, 70.6, 70.6, 70.0, 69.3, 68.4, 55.7, 50.7. HRMS m/z: [M+H]⁺ calculated for [C₂₂H₃₂N₄O₇]⁺ 465.2344, found: 465.2353.

### N-(6-aminopyridin-2-yl)-17-azido-3,6,9,12,15-pentaoxaheptadecanamide (14)

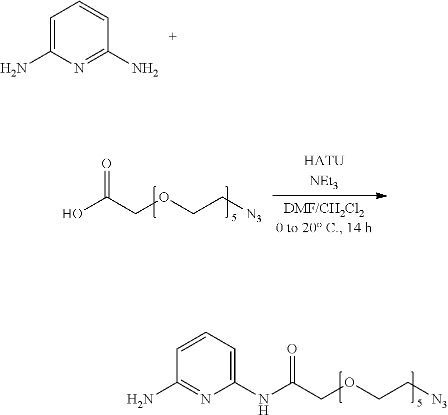

To a solution of 17-azido-3,6,9,12,15-pentaoxaheptadecanoic acid (126 mg, 0.39 mmol, 1 equiv.) in 1 mL of dry CH₂Cl₂ was added HATU (224 mg, 0.59 mmol, 1.5 equiv.), followed by triethylamine (99 mg, 0.23 mmol, 2.5 equiv) dropwise. The solution was cooled to 0° C. under nitrogen atmosphere, incubated for 30 min, then a solution of 1,2-diaminopyridine (193 mg, 1.8 mmol, 4.5 equiv.) in dry CH₂Cl₂/DMF was added. The reaction was allowed to warm to 20° C. and stir for 14 hours. The resulting mixture was diluted with CH₂Cl₂ and washed with saturated sodium carbonate solution (3×20 ml). The combined organic phases were dried over MgSO₄, filtered and concentrated under reduced pressure. The residue was purified by chromatography, eluting with ethyl acetate to yield the title compound as a yellowish oil (100 mg, 81%, 0.24 mmol).

¹H NMR (500 MHz, CDC₃) δ 8.84 (s, 1H), 7.53 (d, J=7.9 Hz, 1H), 7.44 (t, J=7.9 Hz, 1H), 6.25 (dd, J=7.9, 0.7 Hz, 1H), 4.43 (s, 2H), 4.10 (s, 2H), 3.77-3.74 (m, 4H), 3.72-3.70 (m, 4H), 3.69-3.65 (m, 10H), 3.39 (t, J=5.1 Hz, 2H). ¹³C NMR (126 MHz, CDCl₃) δ 168.6, 157.5, 149.4, 140.0, 104.6, 103.5, 71.5, 70.9, 70.7, 70.7, 70.7, 70.4, 70.1, 50.8. HRMS m/z: [M+H]⁺ calculated for [C₁₇H₂₉N₆O₆]⁺ 413.2143, found: 413.2147.

### 17-azido-N-(2-(dimethylamino)ethyl)-3,6,9,12,15-pentaoxaheptadecanamide (15)

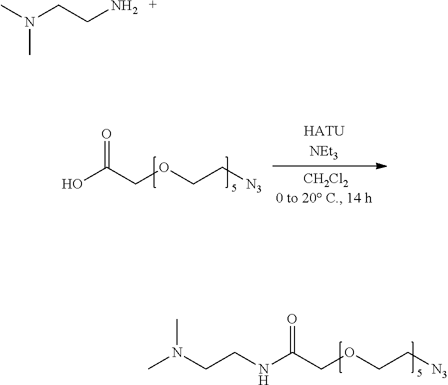

To a solution of 17-azido-3,6,9,12,15-pentaoxaheptadecanoic acid (63 mg, 0.20 mmol, 1.25 equiv.), HATU (119 mg, 0.31 mmol, 2 equiv.) and triethylamine (55 mL, 0.39 mmol, 2.5 equiv.) in dry dichloromethane stirring under a nitrogen atmosphere at 0° C. was added N¹,N¹-dimethylethane-1,2-diamine (17 mL, 0.16 mmol, 1.0 equiv.), dropwise. The resulting mixture was stirred at 0° C. for 30 mins, then warmed to 20° C. and stirred for a further 14 h. The solvent was removed in vacuo and the residue was redissolved in EtOAc (50 mL) and transferred to a separating funnel. Saturated aqueous NaCO₃ (10 mL) and H₂O (40 mL) were added, the funnel shaken, and the layers separated. The aqueous layer was further extracted with EtOAc (2×50 mL), and the combined organic phases were dried over anhydrous MgSO₄ and concentrated. The residue was purified on basic alumina (eluting with a gradient of 0% to 10% MeOH/CH₂Cl₂) to yield the title compound as a colourless oil (25.0 mg, 0.064 mmol, 40%).

¹H NMR (500 MHz, CDCl₃) δ 7.15 (br. s, 1H), 3.98 (s, 2H), 3.67-3.64 (m, 18H), 3.37 (q, J=6.0 Hz, 4H), 2.44 (t, J=6.4 Hz, 2H), 2.25 (s, 6H). ¹³C NMR (126 MHz, CDCl₃) δ 170.0, 71.1, 70.8, 70.8, 70.7, 70.7, 70.7, 70.7, 70.4, 70.1, 58.2, 50.8, 45.4, 36.5. HRMS m/z: [M+H]⁺ calculated for [C₁₆H₃₄N₅O₆]⁺ 392.2504, found: 392.2506.

### 4-((17-azido-3,6,9,12,15-pentaoxaheptadecyl)sulfonyl)phenol (16)

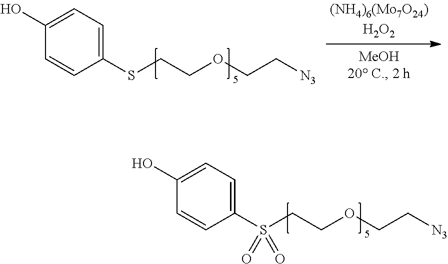

To a solution of 4-((17-azido-3,6,9,12,15-pentaoxaheptadecyl)thio)phenol (12.4 mg, 0.030 mmol, 1 equiv.) in MeOH (2 mL) was added ammonium molybdate hydrate (1.7 mg, 0.006 mmol, 5 mol %) and 30% wt aqueous hydrogen peroxide (12 μL, 0.12 mmol, 4 equiv.). The resultant suspension was stirred at 20° C. for 2 h. Ethyl acetate (5 mL) was added and the mixture was filtered through Celite and concentrated in vacuo. The residue was purified on silica, eluting with 2.5% methanol in dichloromethane to give the title compound as a colourless oil (6.8 mg, 51%, 0.015 mmol).

δ ¹H NMR (400 MHz, CDCl₃) δ 7.76 (d, J=8.8 Hz, 2H), 6.98 (d, J=8.7 Hz, 2H), 3.81-3.78 (m, 2H), 3.75-3.69 (m, 6H), 3.66-3.61 (m, 4H), 3.53-3.51 (m, 2H), 3.38-3.33 (m, 10H). HRMS m/z: [M−H]⁻ calculated for [C₁₈H₂₈N₃O₈S]⁻ 446.1603, found: 446.1602.

### 2-((17-azido-3,6,9,12,15-pentaoxaheptadecyl)thio)phenol (17)

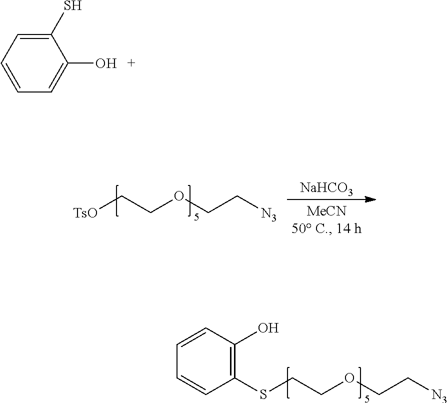

A 4 mL reaction vial was charged with 2-mercaptophenol (30.2 L, 0.30 mmol, 1.0 equiv.) and NaHCO₃ (60.0 mg, 0.60 mmol, 2.0 equiv.) and set under nitrogen. Dry acetonitrile (1 mL) was then added and the mixture was stirred at 25° C. for 1 h. A solution of 17-azido-3,6,9,12,15-pentaoxaheptadecyl 4-methylbenzenesulfonate (143.8 g, 0.312 mmol, 1.04 equiv.) in dry acetonitrile (2 mL) was added and the sealed tube was heated at 50° C. for 16 h. The reaction was cooled, ethyl acetate (20 mL) was added and the mixture was filtered through Celite and concentrated in vacuo. The resultant residue was purified on silica (eluting with 15% acetone/hexanes) to yield the title compound as a colourless oil (14.6 mg, 0.035 mmol, 12%).

¹H NMR (500 MHz, CDCl₃) δ 7.48 (dd, J=7.6, 1.7 Hz, 1H), 7.28-7.24 (m, overlapped with solvent peak, 1H), 6.92 (dd, J=8.2, 1.3 Hz), 6.84 (td, J=7.6, 1.3 Hz), 7.68-7.62 (m, 18H), 3.54 (t, J=6.0 Hz, 2H), 3.38 (t, J=5.1 Hz, 2H), 2.89 (t, J=6.0 Hz, 2H). HRMS m/z: [M−H]⁻ calculated for [C₁₈H₂₈N₃O₆S]⁻ 414.1704, found: 414.1699.

### 2-((17-azido-3,6,9,12,15-pentaoxaheptadecyl)sulfinyl)phenol (18)

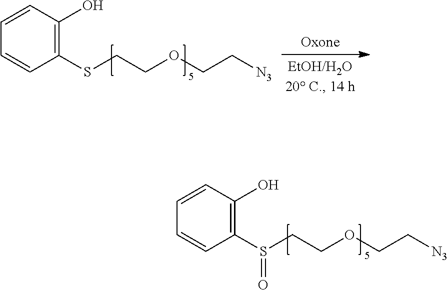

To a solution of 2-((17-azido-3,6,9,12,15-pentaoxaheptadecyl)thio)phenol (25.5 mg, 0.061 mmol, 1 equiv.) in 1:1 H₂O/EtOH (4 mL) was added oxone (23.3 mg, 0.153 mmol, 2.5 equiv.). The resultant suspension was stirred at 20° C. for 14 h. Distilled water was added (50 mL) and the pH was adjusted to 5.0 using 1 M HCl. The aqueous solution was extracted with CHCl₃ (4×50 mL) and the combined organic phases were dried over anhydrous MgSO₄ and concentrated. The residue was purified on silica, eluting with 2% methanol in dichloromethane to give the title compound as a colourless oil (22.3 mg, 85%, 0.052 mmol).

¹H NMR (500 MHz, CDCl₃) δ 7.37-7.33 (m, 1H), 7.16 (dd, J=7.56, 1.4 Hz), 6.93-6.90 (m, 2H), 3.95-3.90 (m, 1H), 3.75-3.71 (m, 1H), 3.66-3.60 (m, 18H), 3.48-3.43 (m, 1H), 3.37 (t, J=5.0 Hz, 2H), 3.23-3.18 (m, 1H); ¹³C NMR (126 MHz, CDCl₃) δ 133.1, 125.8, 121.9, 119.9, 119.4, 70.9, 70.8, 70.8, 70.7, 70.7, 70.7, 70.5, 70.1, 64.1, 55.7, 50.8. HRMS m/z: [M−H]-calculated for [C₁₈H₂₈N₃O₇S]⁻ 430.1653, found: 430.1650.

### 2-((17-azido-3,6,9,12,15-pentaoxaheptadecyl)sulfonyl)phenol (19)

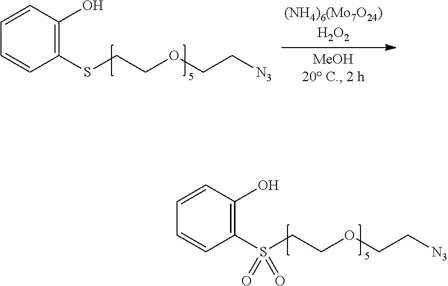

To a solution of 2-((17-azido-3,6,9,12,15-pentaoxaheptadecyl)thio)phenol (19.5 mg, 0.047 mmol, 1 equiv.) in MeOH (2 mL) was added ammonium molybdate hydrate (2.7 mg, 0.002 mmol, 5 mol %) and 30% wt aqueous hydrogen peroxide (19 μL, 0.19 mmol, 4 equiv.). The resultant suspension was stirred at 20° C. for 2 h. Ethyl acetate (5 mL) was added and the mixture was filtered through Celite and concentrated in vacuo. The residue was purified on silica, eluting with 2.5% methanol in dichloromethane to give the title compound as a colourless oil (14 mg, 67%, 0.031 mmol).

¹H NMR (500 MHz, CDCl₃) δ 8.93 (br. s, 1H), 7.66 (dd, J=7.6, 1.7 Hz, 1H), 7.52-7.49 (m, 1H), 7.03-6.99 (m, 2H), 3.85 (t, J=6.0 Hz, 2H), 3.67-3.63 (m, 10H), 3.61-3.59 (m, 2H), 3.55-3.53 (m, 2H), 3.52-3.48 (m, 4H), 3.46-3.44 (m, 2H), 3.37 (t, J=5.1 Hz, 2H); ¹¹C NMR (126 MHz, CDCl₃) δ 156.6, 136.4, 129.5, 122.6, 120.5, 118.9, 70.8, 70.8, 70.8, 70.8, 70.7, 70.7, 70.7, 70.4, 70.2, 64.5, 56.9, 50.8. HRMS m/z: [M−H]⁻ calculated for [C₁₈H₂₈N₃O₈S]⁻ 446.1603, found: 446.1600.

### Illustrative Example 3

### Synthesis and evaluation of PDS-Amimi

Following the procedures outlined in Illustrative Example 1, the compound PDS-Amimi shown below was synthesised and its activity as a degrader was assessed alongside PDS-deg6 (prepared as described in Illustrative Example 1) and the non-degrader control CBX-PDS (prepared as described in Illustrative Example 1).

Synthesis of PDS-Amlmi. N₂,N₆-bis(4-(2-aminoethoxy)quinolin-2-yl)-4-(prop-2-yn-1-yloxy)pyridine-2,6-dicarboxamide (12.4 mg, 20.7 μmol) was dissolved in a 2:1 mixture of H₂O: tBuOH (2.1 mL). A solution of copper sulfate pentahydrate (207 μL, 100 mM, 20.7 μmol) was added followed by a solution of sodium ascorbate (1.07 mL, 100 mM, 107 μmol). The cloudy yellow solution was placed under argon and stirred for 10 min. Solution of 1-(1-(17-azido-3,6,9,12,15-pentaoxaheptadecyl)-1H-imidazol-2-yl)-N,N-dimethylmethanamine (6) (2.9 mL, 10 mM, 29 μmol) was then added. The reaction mixture was stirred at 25° C. for 2 h. The solvent was then removed in vacuo. Then the product purified by HPLC (gradient 100% H₂O, 0.1% FA to 100% MeCN, 0.1% FA). The title compound was obtained as a beige solid (7.2 mg, 7.2 μmol, 35%). HRMS m/z: [M+H]⁺ calculated for [C₅₀H₆₄N₁₃O₁₀]⁺ 1006.4899, found: 1006.4896.

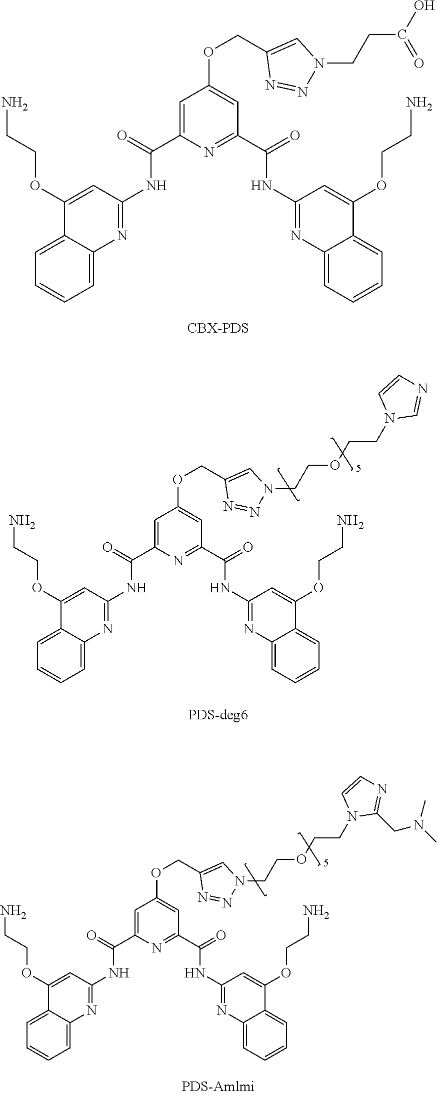

### Assay Protocol

Protocol: G4-forming RNA oligomer (final concentration 200 μM, sequence 5′-UGUGGGAGGGGCGGGUCUGGGUGC-3′) was added to a pH 7.5 HEPES (20 mM) buffer supplemented with KCl (100 mM), MgCl₂ (10 mM). The mixture was heated at 95° C. for 5 minutes, then kept on ice for 30 minutes. CuSO₄ (final concentration 200 μM), THPTA (700 μM) and NaAsc (50 mM) were then added, alongside with CBX-PDS, PDS-deg6 or PDS-Amlmi (200 μM). The reaction mixture was incubated at 37° C. for 4 h, quenched with EDTA (final concentration 12 mM) and then kept at 4° C. The reaction mixtures were analysed by LC-MS.

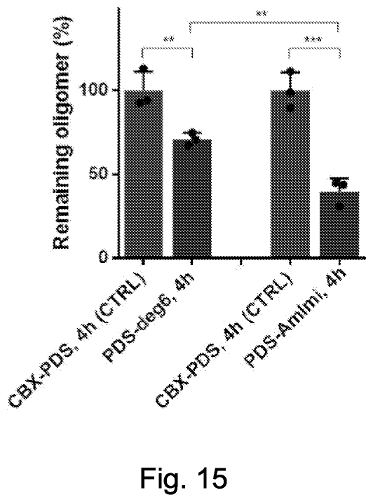

The results are shown in FIG. 15.

It was observed that PDS-Amimi is a more potent degrader than PDS-deg6.

### REFERENCES

A number of publications are cited above in order to more fully describe and disclose the invention and the state of the art to which the invention pertains. Full citations for these references are provided below. The entirety of each of these references is incorporated herein.
Bobbin, et al., “RNA Interference (RNAi)-Based Therapeutics: Delivering on the Promise?”, Annual Review of Pharmacology and Toxicology, 2016, Vol. 56, pp. 103-122.
Cox, et al., “RNA editing with CRISPR-Cas13”, Science, 2017, Vol. 358, pp. 1019-1027.
Gasiunas, et al., “Cas9-crRNA ribonucleoprotein complex mediates specific DNA cleavage for adaptive immunity in bacteria”, Proc. Natl. Acad. Sci. U.S.A., 2012, Vol. 109, E2579-E2586.
Jinek, et al., “A programmable dual-RNA-guided DNA endonuclease in adaptive bacterial immunity”, Science, 2012, Vol. 337, pp. 816-821.
Kelly et al., “Structural and functional conservation of the programmed −1 ribosomal frameshift signal of SARS coronavirus 2 (SARS-CoV-2)”, J. Biol. Chem., 2020, Vol. 295, pp. 10741-10748.
Li. “Minimap2: pairwise alignment for nucleotide sequences”, Bioinformatics, 2018, Vol. 34, pp. 3094-3100.
Mikutis et al., “meCLICK-Seq, a Substrate-Hijacking and RNA Degradation Strategy for the Study of RNA Methylation”, ACS Cent. Sci., 2020, Vol. 6, pp. 2196-2208.
Park et al., “Identification of RNA Pseudoknot-Binding Ligand That Inhibits the −1 Ribosomal Frameshifting of SARS-Coronavirus by Structure-Based Virtual Screening”, J. Am. Chem. Soc., 2011, Vol. 133, pp. 10094-10100.
Santos et al., “G-Quadruplexes and Their Ligands: Biophysical Methods to Unravel G-Quadruplex/Ligand Interactions”, Pharmaceuticals, 2021, Vol. 14, No. 769
Sigman, D. S., et al., Oxygen-dependent cleavage of DNA by the 1,10-phenanthroline. cuprous complex. Inhibition of Escherichia coliDNA polymerase I. J Biol Chem, 1979. 254(24): p. 12269-72.
Tzelepis, et al., “A CRISPR Dropout Screen Identifies Genetic Vulnerabilities and Therapeutic Targets in Acute Myeloid Leukemia”, Cell Reports, 2016, Vol. 17, pp. 1193-1205.
Zamore, et al., “RNAi: Double-Stranded RNA Directs the ATP-Dependent Cleavage of mRNA at 21 to 23 Nucleotide Intervals”, Cell, 2000, Vol. 101, pp. 25-33.
Zhao, C.; Qin, G.; Niu, J.; Wang, Z.; Wang, C.; Ren, J.; Qu, X., “Targeting RNA G-Quadruplex in SARS-CoV-2: A Promising Therapeutic Target for COVID-197”, Angew. Chem. Int. Ed., 2021, 60 (1), 432-438.
Ziv, et al., “The Short- and Long-Range RNA-RNA Interactome of SARS-CoV-2.” Mol. Cell., 2020, Vol. 80, pp. 1067-1077.

1. A bifunctional compound of formula (I) or a pharmaceutically acceptable salt or solvate thereof: 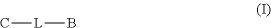
wherein:
C is a cleavage group as defined herein;
L is a linker; and
B is a non-covalent binding group
with the proviso that C is not:
(i) an imidazole (1,3-diazole) group that is optionally substituted with one, two or three (1-6C)alkyl groups, which may be the same or different; or
(ii) a nucleic acid cleavage group of the formula Z: 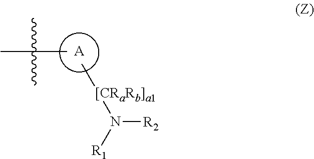
wherein: [symbol]
denotes the point of attachment to L;
Ring A is absent or a nitrogen-containing heteroaryl or heterocyclic ring which is optionally further substituted with one or more substituent groups selected from halo, trifluoromethyl, trifluoromethoxy, cyano, hydroxyl, (1-4C)alkyl, (1-4C)haloalkyl, (1-4C)hydroxyalkyl, OR_{c}, C(O)R_{c}, C(O)OR_{c}, OC(O)R_{c}, C(O)N(R_{d})R_{c}, N(R_{d})C(O)R_{c}, S(O)_{y}R, (wherein y is 0, 1 or 2), SO₂N(R_{d})R_{c}, N(R_{d})SO₂R_{c}, or NR_{c}R_{d}, wherein R_{c} and R_{d} are selected from hydrogen or (1-4C)alkyl;
integer a1 is 0, 1, 2 or 3;
Ra and Rb are each independently selected at each occurrence from hydrogen or (1-2C)alkyl;
R₁ and R₂ are each independently selected from hydrogen, (1-6C)alkyl, heterocyclic ring, heterocycle-(1-3C)alkyl, heteroaryl, heteroaryl-(1-3C)alkyl, (3-6C)cycloalkyl, or (3-6C)cycloalkyl-(1-3C)alkyl, each of which is optionally substituted with one or more substituent groups selected from halo, trifluoromethyl, trifluoromethoxy, cyano, hydroxyl, ORₑ, C(O)Rₑ, C(O)ORₑ, OC(O)Rₑ, C(O)N(R_{f})Rₑ, N(R_{f})C(O)Rₑ, S(O)_{y}Rₑ (wherein y is 0, 1 or 2), SO₂N(R_{f})Rₑ, N(R_{f})SO₂Rₑ, or NRₑR_{f}, wherein Rₑ and R_{f} are selected from hydrogen or (1-4C)alkyl, or
R₁ and R₂ are linked such that, together with the atoms to which they are attached, they form a 4-6 membered heterocycle or a 5- or 6-membered heteroaryl, wherein any 4-6 membered heterocycle or 5- or 6-membered heteroaryl is optionally substituted with one or more substituent groups selected from halo, trifluoromethyl, trifluoromethoxy, cyano, hydroxyl, (1-4C)alkyl, (1-4C)haloalkyl, (1-4C)hydroxyalkyl, OR_{g}, C(O)R_{g}, C(O)OR_{g}, OC(O)R_{g}, C(O)N(Rₕ)R_{g}, N(Rₕ)C(O)R_{g}, S(O)_{y}R_{g} (wherein y is 0, 1 or 2), SO₂N(Rₕ)R_{g}, N(Rₕ)SO₂R_{g}, or NR_{g}Rₕ, wherein R_{g} and Rₕ are selected from hydrogen or (1-4C)alkyl;
wherein, when ring A is absent, R₁ and R₂ are each independently selected from hydrogen, heterocyclic ring, heterocycle-(1-3C)alkyl, heteroaryl, heteroaryl-(1-3C)alkyl, (3-6C)cycloalkyl, or (3-6C)cycloalkyl-(1-3C)alkyl, each of which is optionally substituted with one or more substituent groups selected from halo, trifluoromethyl, trifluoromethoxy, cyano, hydroxyl, ORₑ, C(O)Rₑ, C(O)ORₑ, OC(O)Rₑ, C(O)N(R_{f})Rₑ, N(R_{f})C(O)Rₑ, S(O)_{y}Rₑ (wherein y is 0, 1 or 2), SO₂N(R_{f})Rₑ, N(R_{f})SO₂Rₑ, or NRₑR_{f}, wherein Rₑ and R_{f} are selected from hydrogen or (1-4C)alkyl, provided that R₁ and R₂ are not both hydrogen.

2. A bifunctional compound of formula (I) or a pharmaceutically acceptable salt or solvate thereof, according to claim 1, wherein C comprises a basic nitrogen atom or hydroxy group that either:
(i) has a pKa within the range of 5.5 to 9, or 6 to 9, or 6.2 to 8.6; and/or
(ii) is a nitrogen atom or hydroxy group that is capable of forming chelation complex with a metal (e.g. copper or zinc).

3. A bifunctional compound of formula (I) or a pharmaceutically acceptable salt or solvate thereof, according to claim 1 or claim 2, wherein the cleavage group C is selected from:
(i) any N or C—OH containing moiety whereby at least one N or C—OH group has a pKa within the range of 5.5 to 9, or 6 to 9, or 6.2 to 8.6;
(ii) any N or OH containing moiety that is capable of chelating to copper at physiological pH.

4. A bifunctional compound of formula (I) or a pharmaceutically acceptable salt or solvate thereof, according to any one of claims 1 to 3, wherein the cleavage group C is a group of the formula: 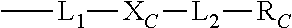
wherein:
L₁ is absent or (1-2C)alkylene;
X_{C} is absent or selected from —O—, —S—, —SO—, —SO₂—, —N(R_{XC1})—, —C(O)—, —C(O)O—, —OC(O)—, —C(O)N(R_{XC1})—, —N(R_{XC1})C(O)—, —S(O)₂N(R_{XC1}), or —N(R_{XC1})SO₂—; and
wherein R_{XC1} is selected from hydrogen or (1-6C)alkyl, (3-6C)cycloalkyl, (3-6C)cycloalkyl(1-2C)alkylene, —(CH₂)ₘ₁-aryl, —(CH₂)ₘ₁-heteroaryl or —(CH₂)ₘ₁-heterocyclic ring, wherein m1 is 0 to 2;
L₂ is absent or (1-2C)alkylene;
R_{C} is selected from hydrogen, (1-4C)alkyl, (3-6C)cycloalkyl, phenyl, heteroaryl or heterocyclyl;
wherein:
an alkyl, cycloalkyl, phenyl, is substituted by one or more R_{A} substituents and is optionally further substituted by one or more R_{B} substituents;
a heteroaryl or heterocyclic ring is optionally substituted by one or more R_{A} and/or R_{B} substituents;
R_{A} is a group selected from:
(i) —OH;
(ii) NR_{A1}R_{A2};
(iii) -(1-6C)alkylene-NR_{A1}R_{A2};
wherein:
R_{A1} and R_{A2} are each independently selected from hydrogen, (1-6C)alkyl, or (1-6C)heteroalkyl;
R_{B} is selected from halo, nitro, cyano, R_{BA}, —[CH₂]ₜ—OR_{BA}, —[CH₂]ₜ—C(O)R_{BA}, —[CH₂]ₜ—C(O)OR_{BA}, —[CH₂]ₜ—OC(O)R_{BA}, —[CH₂]ₜ—C(O)N(R_{BB})R_{BA}, —[CH₂]ₜ—N(R_{BB})C(O)R_{BA}, —[CH₂]ₜ—S(O)ₚR_{BA} (where p is 0, 1 or 2), —[CH₂]ₜ—SO₂N(R_{BB})R_{BA}, or —[CH₂]ₜ—N(R_{BB})SO₂R_{BA};
wherein t is 0, 1, 2 or 3;
R_{BA} is hydrogen or (1-4C)alkyl optionally substituted by halo, hydroxy, amino or cyano; and
R_{BB} is hydrogen or (1-2C)alkyl;
wherein the cleavage group is not:
a) an imidazole (1,3-diazole) group that is optionally substituted with one, two or three (1-6C)alkyl groups, which may be the same or different; or
b) a group of the formula Z defined above.

5. A bifunctional compound of formula (I) or a pharmaceutically acceptable salt or solvate thereof, according to any one of claims 1 to 4, wherein the cleavage group C is a group of the formula: 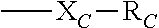
wherein:
X_{C} is absent or selected from —O—, —S—, —SO—, —SO₂—, —N(R_{XC1})—, —C(O)—, —C(O)N(R_{XC1})— or —N(R_{XC1})C(O)—; and wherein R_{XC1} is selected from hydrogen or (1-6C)alkyl or —(CH₂)ₘ₁-heteroaryl, wherein m1 is 0 to 2;
R_{C} is selected from hydrogen, (1-4C)alkyl, phenyl, heteroaryl or heterocyclyl;
wherein:
the alkyl, cycloalkyl, phenyl, is substituted by one or more R_{A} substituents and is optionally further substituted by one or more R_{B} substituents;
a heteroaryl or heterocyclic ring is optionally substituted by one or more R_{A} and/or R_{B} substituents;
R_{A} is a group selected from:
(iv) —OH;
(v) NR_{A1}R_{A2};
(vi) -(1-6C)alkylene-NR_{A1}R_{A2};
wherein:
R_{A1} and R_{A2} are each independently selected from hydrogen, (1-6C)alkyl, or (1-6C)heteroalkyl;
R_{B} is selected from halo, nitro, cyano, R_{BA}—[CH₂]ₜ—OR_{BA}, or —[CH₂]ₜ—C(O)OR_{BA},
wherein t is 0, 1, 2 or 3; and
R_{BA} is (1-4C)alkyl;
wherein the cleavage group is not:
c) an imidazole (1,3-diazole) group that is optionally substituted with one, two or three (1-6C)alkyl groups, which may be the same or different; or
d) a group of the formula Z defined above.

6. A bifunctional compound of formula (I) or a pharmaceutically acceptable salt or solvate thereof, according to any one of claims 1 to 5, wherein the cleavage group C is selected from a group of the formula:

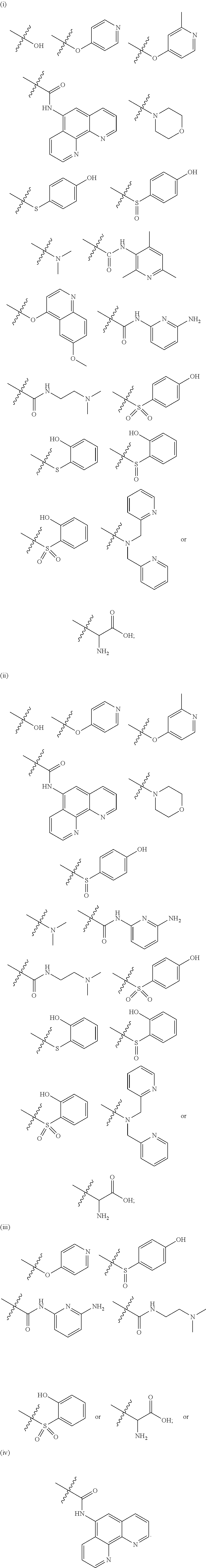

7. A bifunctional compound of formula (I) or a pharmaceutically acceptable salt or solvate thereof, according to any one of claims 1 to 6, wherein the linker group L is selected from a group of the formula (L-I), (L-II), (L-III) or (L-IV) shown below: 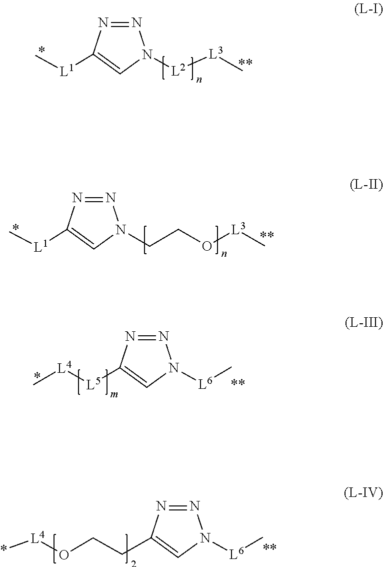
wherein:
L¹ is a covalent bond or a (1-6C)alkylene group or (1-6C)heteroalkylene;
L² is a (1-6C)alkylene group or a (1-6C)heteroalkylene group;
L³ is a (1-6C)alkylene group;
n is 0 to 8;
L⁴ is a (1-6C)alkylene group;
L⁵ is a (1-6C)alkylene group or a (1-6C)heteroalkylene group;
L⁶ is a covalent bond or a (1-2C)alkylene group;
m is 1 to 8;
* is the attachment point with the non-covalent binding group (—B); and
** is the attachment point with the cleavage group (—C).

8. A bifunctional compound of formula (I) or a pharmaceutically acceptable salt or solvate thereof, according to claim 7, wherein L¹ is a covalent bond or methylene and L⁶ is a covalent bond or methylene.

9. A bifunctional compound of formula (I) or a pharmaceutically acceptable salt or solvate thereof, according to claim 7 or 8, wherein L³ is (1-4C)alkylene and L⁴ is (1-4C)alkylene.

10. A bifunctional compound of formula (I) or a pharmaceutically acceptable salt or solvate thereof, according to any one of claims 7 to 9, wherein L³ and L⁴ are ethylene.

11. A bifunctional compound of formula (I) or a pharmaceutically acceptable salt or solvate thereof, according to any one of claims 7 to 10, wherein m and n are 2 to 5.

12. A bifunctional compound of formula (I) or a pharmaceutically acceptable salt or solvate thereof, according to any one of claims 7 to 11, wherein L² and L⁵ are selected from ethylene oxide (—CH₂CH₂O—), propylene oxide (—CH₂CH₂CH₂O—) and tetramethylene oxide (—CH₂CH₂CH₂CH₂O—).

13. A bifunctional compound of formula (I) or a pharmaceutically acceptable salt or solvate thereof, according to any one of claims 7 to 12, wherein L² and L⁵ are ethylene oxide (—CH₂CH₂O—).

14. A bifunctional compound of formula (I) or a pharmaceutically acceptable salt or solvate thereof, according to any one of claims 1 to 13, wherein B is selected from an oligonucleotide, nanobody, antibody, antibody fragment or small molecule that is capable of binding to a nucleic acid; or B is a group of formula (B-1), (B-II), (B—III) or (B—IV) shown below: 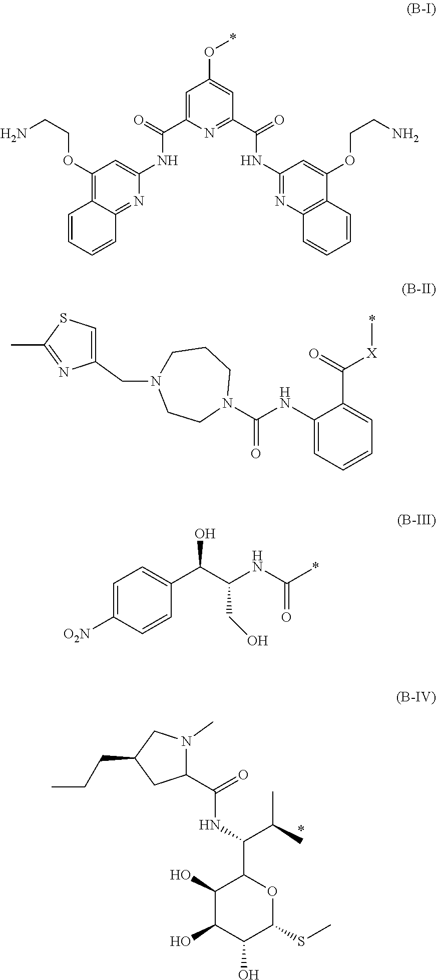
wherein:
X is O or NH.

15. A bifunctional compound of formula (I) or a pharmaceutically acceptable salt or solvate thereof, according to any one of claims 1 to 14, wherein the compound of formula (I) is selected from a group of formula (II), (Ill), (IV) or (V) shown below: 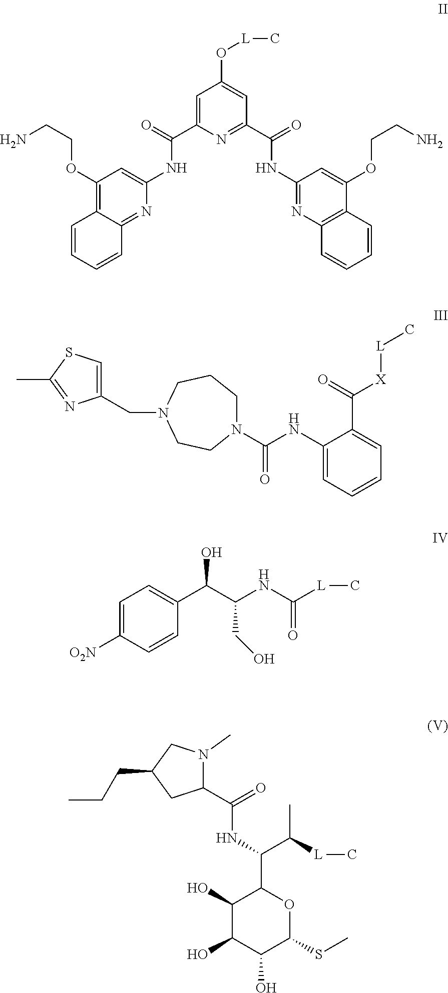
wherein L and C are each as defined in any one of claims 1 to 14 above and X is NH or O.

16. A pharmaceutical composition comprising a bifunctional compound, or a pharmaceutically acceptable salt or solvate thereof, according to any one of claims 1 to 15 and one or more pharmaceutically acceptable excipients.

17. A bifunctional compound, or a pharmaceutically acceptable salt or solvate thereof, according to any one of claims 1 to 15, or a pharmaceutical composition according to claim 16, for use as a medicament.

18. A bifunctional compound, or a pharmaceutically acceptable salt or solvate thereof, according to any one of claims 1 to 15, or a pharmaceutical composition according to claim 16, for use in the treatment of a proliferative disorder (e.g. cancer) or a bacterial or viral infection.

19. A method of treating a proliferative disorder (e.g. cancer) or a bacterial or viral infection, the method comprising administering a therapeutically effective dose of a bifunctional compound, or a pharmaceutically acceptable salt or solvate thereof, according to any one of claims 1 to 15, or a pharmaceutical composition according to claim 16.

20. Use of a bifunctional compound, or a pharmaceutically acceptable salt or solvate thereof, according to any one of claims 1 to 15, or a pharmaceutical composition according to claim 16, for epigenetic and epitranscriptomic analysis/mapping.

21. A method for cleaving a target nucleic acid molecule, the method comprising:
contacting the target nucleic acid molecule with a bifunctional compound, or a salt or solvate thereof, according to any one of claims 1 to 15 such that the compound non-covalently binds to the target nucleic acid molecule; and
allowing the compound to cleave the target nucleic acid molecule bound thereto.

22. A method for identifying a secondary or tertiary structure within a target nucleic acid molecule, the method comprising:
providing first and second populations of nucleic acid molecules, each population comprising the target nucleic acid molecule;
introducing into the first population of nucleic acid molecules a bifunctional compound, or a salt or solvate thereof, according to any one of claims 1 to 15;
allowing the bifunctional compound of the present invention to cleave the target nucleic acid molecule present in the first population; and
identifying nucleic acid molecules which are present in a reduced amount in the first population relative to the second population.
